(19) United States
(12) Patent Application Publication
(10) Pub. No.: US 2024/0059675 A1
(43) Pub. Date: Feb. 22, 2024

(54) PIPERIDINYLPYRIDINYLCARBONITRILE DERIVATIVES AS INHIBITORS OF GLUTAMINYL-PEPTIDE CYCLOTRANSFERASE AND GLUTAMINYL-PEPTIDE CYCLOTRANSFERASE LIKE PROTEIN

(71) Applicant: Boehringer Ingelheim International GmbH, Ingelheim am Rhein (DE)

(72) Inventors: Jens WILLWACHER, Biberach an der Riss (DE); Florian Paul Christian BINDER, Maselheim (DE); Georg DAHMANN, Biberach an der Riss (DE); James Young Soo YANG HAMILTON, Biberach an der Riss (DE); Sandra Ruth HANDSCHUH, Biberach an der Riss (DE); Sophia Astrid REINDL, Mittelbiberach (DE)

(21) Appl. No.: 18/362,034

(22) Filed: Jul. 31, 2023

(30) Foreign Application Priority Data
Aug. 3, 2022 (EP) 22188580.9

(51) Int. Cl. C07D 401/14; C07D 417/14; C07D 471/04; C07D 487/04

(52) U.S. Cl. CPC C07D 401/14; C07D 417/14; C07D 471/04; C07D 487/04

(57) ABSTRACT

The present disclosure provides certain piperidinylpyridinylcarbonitrile derivatives, and pharmaceutically acceptable salts thereof, that are inhibitors of Glutaminyl-peptide cyclotransferase (QPCT) and glutaminyl-peptide cyclotransferase-like protein (QPCTL), and are therefore useful for the treatment of diseases treatable by inhibition of QPCT/L. Also provided are pharmaceutical compositions containing the same, and processes for preparing said compounds.

### RELATED APPLICATIONS

This application is a nonprovisional patent application which claims priority under 35 U.S.C. 119(b) and 37 CFR 1.55 to pending EP Serial No. 22188580.9, filed Aug. 3, 2022, which is incorporated herein by reference in its entirety.

### TECHNICAL FIELD

The present disclosure provides certain piperidinylpyridinylcarbonitrile derivatives, and pharmaceutically acceptable salts thereof, that are inhibitors of Glutaminyl-peptide cyclotransferase (QPCT) and glutaminyl-peptide cyclotransferase-like protein (QPCTL), and are therefore useful for the treatment of diseases treatable by inhibition of QPCT/L. Also provided are pharmaceutical compositions containing the same, and processes for preparing said compounds.

### BACKGROUND INFORMATION

Glutaminyl-peptide cyclotransferase (QPCT) and glutaminyl-peptide cyclotransferase-like protein (QPCTL) catalyze the intramolecular cyclization of N-terminal glutamine (Q) residues into pyroglutamic acid (pE) liberating ammonia [Stephan Schilling et al., “Identification of Human Glutaminyl Cyclase as a Metalloenzyme POTENT INHIBITION BY IMIDAZOLE DERIVATIVES AND HETEROCYCLIC CHELATORS,” Journal of Biological Chemistry 278, no. 50 (2003): 49773-79, https://doi.org/10.1074/jbc.m309077200; Holger Cynis et al., “Isolation of an Isoenzyme of Human Glutaminyl Cyclase: Retention in the Golgi Complex Suggests Involvement in the Protein Maturation Machinery,” Journal of Molecular Biology 379, no. 5 (2008): 966-80, https://doi.org/10.1016/j.jmb.2008.03.078; Anett Stephan et al., “Mammalian Glutaminyl Cyclases and Their Isoenzymes Have Identical Enzymatic Characteristics,” FEBS Journal 276, no. 22 (2009): 6522-36, https://doi.org/10.1111/j.1742-4658.2009.07337.x.]. While QPCT is a secreted protein, QPCTL is retained within the Golgi complex. Both enzymes share a high homology in the active site and similar catalytic specificity. Because of the high homology in the active site, inhibition of the active site blocks the enzymatic activity of both enzymes: QPCT and QPCTL. Hence the term “QPCT/L” describes both enzymes at once. Due to their different cellular localisation, differences in their relevance for modification of biological substrates have been reported. Known substrates of the intracellular QPCTL and/or extracellular QPCT are CD47 [Meike E. W. Logtenberg et al., “Glutaminyl Cyclase Is an Enzymatic Modifier of the CD47− SIRPα Axis and a Target for Cancer Immunotherapy,” Nature Medicine 25, no. 4 (2019): 612-19, https://doi.org/10.1038/s41591-019-0356-z.], different chemokines (like for example CCL2 and 7 or CX3CL1) [Rosa Barreira da Silva et al., “Loss of the Intracellular Enzyme QPCTL Limits Chemokine Function and Reshapes Myeloid Infiltration to Augment Tumor Immunity,” Nature Immunology 23, no. 4 (2022): 568-80, https://doi.org/10.1038/s41590-022-01153-x; Astrid Kehlen et al., “N-Terminal Pyroglutamate Formation in CX3CL1 Is Essential for Its Full Biologic Activity,” Bioscience Reports 37, no. 4 (2017): BSR20170712, https://doi.org/10.1042/bsr20170712.], Amyloid-b peptides [Cynis et al., “Isolation of an Isoenzyme of Human Glutaminyl Cyclase: Retention in the Golgi Complex Suggests Involvement in the Protein Maturation Machinery.” ] or hormones like TRH [Andreas Becker et al., “IsoQC (QPCTL) Knock-out Mice Suggest Differential Substrate Conversion by Glutaminyl Cyclase Isoenzymes,” Biological Chemistry 397, no. 1 (2016): 45-55, https://doi.org/10.1515/hsz-2015-0192.]. The modification of N-terminal glutamine to pyroglutamate on the substrates has functional consequences for the proteins and could impact different pathomechanisms in several diseases. CD47 is expressed on the cell surface of virtually all cells of the body, including apoptotic cells, senescent cells or cancer cells. [Meike E. W. Logtenberg, Ferenc A. Scheeren, and Ton N. Schumacher, “The CD47-SIRPα Immune Checkpoint,” Immunity 52, no. 5 (2020): 742-52, https://doi.org/10.1016/j.immuni.2020.04.011]. The main ligand for CD47 is signal-regulatory protein alpha (SIRPα), an inhibitory transmembrane receptor present on myeloid cells, such as macrophages, monocytes, neutrophils, dendritic cells and others. QPCTL mediated N-terminal pyroglutamate modification on CD47 is required for SIRPα binding [Deborah Hatherley et al., “Paired Receptor Specificity Explained by Structures of Signal Regulatory Proteins Alone and Complexed with CD47,” Molecular Cell 31, no. 2 (2008): 266-77, https://doi.org/10.1016/j.molcel.2008.05.026; Meike E. W. Logtenberg et al., “Glutaminyl Cyclase Is an Enzymatic Modifier of the CD47− SIRPα Axis and a Target for Cancer Immunotherapy,” Nature Medicine 25, no. 4 (2019): 612-19, https://doi.org/10.1038/s41591-019-0356-z.] This signaling axis induces a “Don't Eat Me Signal”, preventing engulfment of CD47 expressing cells by macrophages. Thus, high expression of CD47 is connected to the pathogenesis of cancer [Logtenberg et al., “Glutaminyl Cyclase Is an Enzymatic Modifier of the CD47− SIRPα Axis and a Target for Cancer Immunotherapy,” 2019; Meike E. W. Logtenberg, Ferenc A. Scheeren, and Ton N. Schumacher, “The CD47-SIRPα Immune Checkpoint,” Immunity 52, no. 5 (2020): 742-52, https://doi.org/10.1016/j.immuni.2020.04.011.], COVID-19 [Katie-May McLaughlin et al., “A Potential Role of the CD47/SIRPalpha Axis in COVID-19 Pathogenesis,” Current Issues in Molecular Biology 43, no. 3 (2021): 1212-25, https://doi.org/10.3390/cimb43030086.], lung fibrosis [Gerlinde Wernig et al., “Unifying Mechanism for Different Fibrotic Diseases,” Proceedings of the National Academy of Sciences 114, no. 18 (2017): 4757-62, https://doi.org/10.1073/pnas.1621375114; Lu Cui et al., “Activation of JUN in Fibroblasts Promotes Pro-Fibrotic Programme and Modulates Protective Immunity,” Nature Communications 11, no. 1 (2020): 2795, https://doi.org/10.1038/s41467-020-16466-4.], systemic sclerosis [Wernig et al., “Unifying Mechanism for Different Fibrotic Diseases”; Tristan Lerbs et al., “CD47 Prevents the Elimination of Diseased Fibroblasts in Scleroderma,” JCI Insight 5, no. 16 (2020): e140458, https://doi.org/10.1172/jci.insight.140458.] and liver fibrosis [Taesik Gwag et al., “Anti-CD47 Antibody Treatment Attenuates Liver Inflammation and Fibrosis in Experimental Non-alcoholic Steatohepatitis Models,” Liver International 42, no. 4 (2022): 829-41, https://doi.org/10.1111/liv.15182.]. Since enhanced CD47 expression blocks the clearance of apoptotic cells, there is an accrual of apoptotic lung epithelial cells, leading to a profibrotic stimulus and accelerating lung inflammation and -scaring [Alexandra L. McCubbrey and Jeffrey L. Curtis, “Efferocytosis and Lung Disease,” Chest 143, no. 6 (2013): 1750-57, https://doi.org/10.1378/chest.12-2413; Brennan D. Gerlach et al., “Efferocytosis Induces Macrophage Proliferation to Help Resolve Tissue Injury,” Cell Metabolism, 2021, https://doi.org/10.1016/j.cmet.2021.10.015.]. Since CD47 half-life and function is majorly dependent on QPCTL enzyme activity, QPCT and QPCTL inhibition could be a suitable mechanism as a treatment in lung fibrosis such as IPF or SSC-ILD [Lerbs et al., “CD47 Prevents the Elimination of Diseased Fibroblasts in Scleroderma.” ], alone or together with current standard of care in pulmonary fibrosis like Nintedanib [Luca Richeldi et al., “Efficacy and Safety of Nintedanib in Idiopathic Pulmonary Fibrosis,” The New England Journal of Medicine 370, no. 22 (2014): 2071-82, https://doi.org/10.1056/nejmoal402584; Kevin R Flaherty et al., “Nintedanib in Progressive Fibrosing Interstitial Lung Diseases,” New England Journal of Medicine 381, no. 18 (2019): 1718-27, https://doi.org/10.1056/nejmoa1908681.] or future treatments like a PDE4 inhibitor [Luca Richeldi et al., “Trial of a Preferential Phosphodiesterase 4B Inhibitor for Idiopathic Pulmonary Fibrosis,” New England Journal of Medicine 386, no. 23 (2022): 2178-87, https://doi.org/10.1056/nejmoa2201737].

By expression of CD47, cancer cells can evade destruction by the immune system or evade immune surveillance, e.g. by evading phagocytosis by immune cells [Stephen B. Willingham et al., “The CD47-Signal Regulatory Protein Alpha (SIRPα) Interaction Is a Therapeutic Target for Human Solid Tumors,” Proceedings of the National Academy of Sciences 109, no. 17 (2012): 6662-67, https://doi.org/10.1073/pnas.1121623109].

In addition to CD47, chemokines, such as CCL2 and CX3CL1, have been identified as QPCTL and/or QPCT substrates [Holger Cynis et al., “The Isoenzyme of Glutaminyl Cyclase Is an Important Regulator of Monocyte Infiltration under Inflammatory Conditions,” EMBO Molecular Medicine 3, no. 9 (2011): 545-58, https://doi.org/10.1002/emmm.201100158]. The formation of the N-terminal pGlu was shown to increase in vivo activity, both by conferring resistance to aminopeptidases and by increasing its capacity to induce chemokine receptor signaling. Two main monocyte chemoattractants CCL2 and CCL7 are insensitive to DPP4-inactivation in vivo because of an intracellular mechanism of N-terminal cyclization mediated by the Golgi-associated enzyme QPCTL. It has been shown that QPCTL is a critical regulator of monocyte migration into solid tumors [Kaspar Bresser et al., “QPCTL Regulates Macrophage and Monocyte Abundance and Inflammatory Signatures in the Tumor Microenvironment,” Oncoimmunology 11, no. 1 (2022): 2049486, https://doi.org/10.1080/2162402x.2022.2049486; Rosa Barreira da Silva et al., “Loss of the Intracellular Enzyme QPCTL Limits Chemokine Function and Reshapes Myeloid Infiltration to Augment Tumor Immunity,” Nature Immunology, 2022, 1-13, https://doi.org/10.1038/s41590-022-01153-x]. Targeting of chemokines has long been pursued as a potential strategy for modulating cellular trafficking in different disease settings.

It is therefore desirable to provide potent QPCT/L inhibitors.

Jimenez-Sanchez, et al., Nature Chemical Biology, 2015, 11, 347-357, (hereinafter “J-S, NCB 2015”) discloses the human glutaminyl cyclase (hQC) inhibitors SEN177 and SEN180:

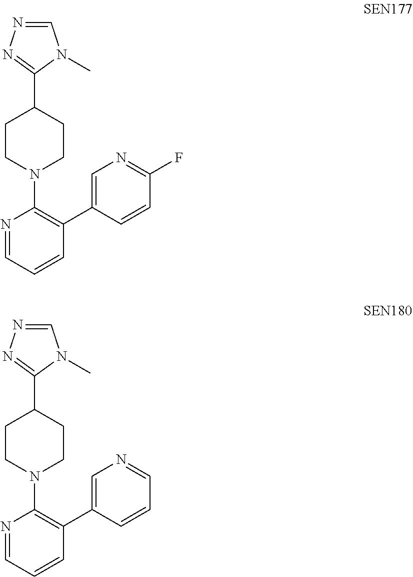 [SMILES: Cn1cnnc1C1CCN(c2ncccc2-c2ccc(F)nc2)CC1 ; Cn1cnnc1C1CCN(c2ncccc2-c2cccnc2)CC1]

SEN177 is disclosed therein (supplementary information) as having a IC₅₀ on isolated hQC of 53 nM and on isolated QPCTL of 13 nM. SEN180 is disclosed therein (supplementary information) as having a IC₅₀ on hQC of 170 nM and on QPCTL of 58 nM.

Pozzi, C, et al, Journal of Biological Inorganic Chemistry, 2018, 23, (8), 1219-1226, (hereinafter “P, JBIC 2018”), further discloses SEN177 and its binding mode within the hQC cavity. SEN177 is disclosed therein as having a Ki on isolated hQC of 20 nM.

WO 2018/178384 discloses QPCTL inhibitors of the general formula A-B-D-E, which include examples 1094 and 1095 (Formula (XIIa) on page 123 and table on page 125):

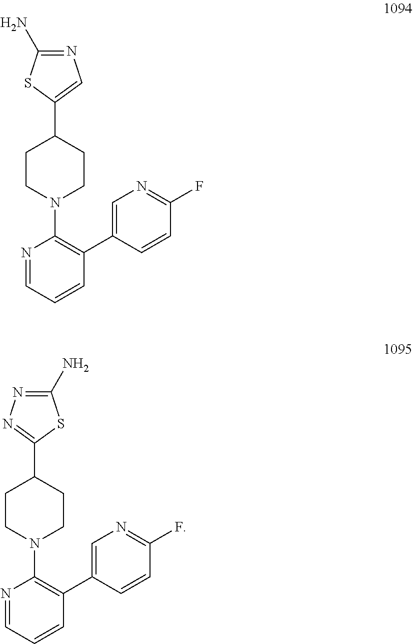

WO 2018/178384 does not disclose any biological data for examples 1094 or 1095.

### DETAILED DESCRIPTION OF THE INVENTION

The present invention discloses novel piperidinylpyridinylcarbonitrile derivatives of formula (I)

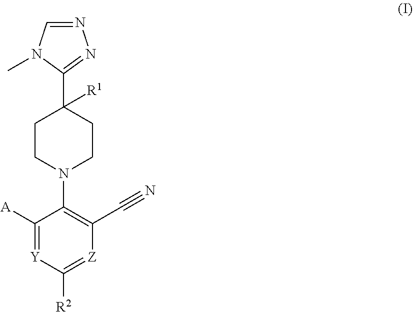

that are inhibitors of Glutaminyl-peptide cyclotransferase (QPCT) and glutaminyl-peptide cyclotransferase-like protein (QPCTL), possessing appropriate pharmacological and pharmacokinetic properties enabling their use as medicaments for the treatment of conditions and/or diseases treatable by inhibition of QPCT/L.

The compounds of the present invention may provide several advantages, such as enhanced potency, cellular potency, high metabolic and/or chemical stability, high selectivity, safety and tolerability, enhanced solubility, enhanced permeability, desirable plasma protein binding, enhanced bioavailability, suitable pharmacokinetic profiles, and the possibility to form stable salts.

### Compounds of the Invention

The present invention provides novel piperidinylpyridinylcarbonitrile derivatives that surprisingly, are potent inhibitors of QPCT and QPCTL (Assay A), as well as potent inhibitors of QPCT/L in cells relevant for, but not limited to, lung diseases or cancer, (Assay B). Furthermore, the present novel piperidinylpyridinylcarbonitrile derivatives have appropriate membrane permeability and a low in vitro efflux (Assay C).

Consequently, compounds of the present invention are more viable for human use.

Compounds of the present invention differ structurally from SEN177 in Pozzi, C, et al, Journal of Biological Inorganic Chemistry, 2018, 23, (8), 1219-1226, in that the pyridinyl ring attached to the piperidinyl ring contains a ring-nitrogen which is in either of two meta-positions to the piperidinyl ring attachment position. Furthermore, a carbonitrile substituent is attached at the ortho-position to the piperidinyl ring attachment position of said pyridinyl ring. Still further, R¹ and R² are not limited to hydrogen or methyl and A represents heterocyclic ring systems beyond pyridinyl.

Compounds of the present invention differ structurally from examples 1094 and 1095 in WO 2018/178384 in that the pyridinyl ring attached to the piperidinyl ring contains a ring-nitrogen which is in either of two meta-positions to the piperidinyl ring attachment position. Furthermore, a carbonitrile substituent is attached at the ortho-position to the piperidinyl ring attachment position of said pyridinyl ring. Still further, R¹ and R² are not limited to hydrogen and A represents heterocyclic ring systems beyond pyridinyl. Still further, the 5-membered heterocyclic ring attached to the piperidinyl ring at the 4-position relative to the piperidinyl nitrogen is in example 1094 an aminothiazolyl ring and in example 1095 an aminothiadiazolyl ring whereas in compounds of the present invention it is a 3-substituted-4-methyl-4H-1,2,4-triazolyl ring.

These structural differences between compounds of the present invention and the prior art unexpectedly lead to a favourable combination of (i) potent inhibition of QPCT and QPCTL, (ii) potent inhibition of QPCT/L in cells relevant for, but not limited to, lung diseases or cancer, and (iii) appropriate membrane permeability and a low in vitro efflux.

Compounds of the invention are thus superior to those disclosed in the prior art in terms of the combination of the following parameters:
potent inhibition of QPCT and QPCTL (Assay A)
potent inhibition of QPCT/L in cells relevant for, but not limited to, lung diseases or cancer (Assay B)
appropriate membrane permeability and a low in vitro efflux (Assay C)

The present invention provides novel compounds according to formula (I)

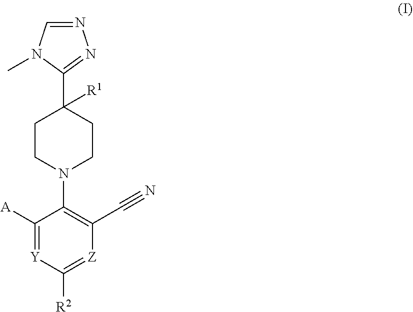

wherein
Y is N and Z is R⁴C or Y is HC and Z is N;
A is A1a which is a 5- or 6-membered mono-heteroaryl ring containing one nitrogen or containing two heteroatom members wherein one is nitrogen and the second is selected from nitrogen or sulphur;
or A is A1b which is a 9- or 10-membered fused bicyclic-heteroaryl ring containing one to four nitrogens; and wherein A1a or A1b is independently substituted with one or two R³;
R¹ is selected from the group R1a, consisting of H, C₁₋₄-alkyl and halo;
R² is selected from the group R2a, consisting of halo, H, C₁₋₄-alkyl, C₃₋₄-cycloalkyl and F₁₋₉-fluoro-C₁₋₄-alkyl;
R³ is selected from the group R3a, consisting of H, halo, C₁₋₄-alkyl, C₃₋₄-cycloalkyl, C₃₋₄-fluorocycloalkyl, F₁₋₉-fluoro-C₁₋₄-alkyl, C₁₋₄-alkyloxy, C₃₋₄-cycloalkyloxy and pyrazolyl,

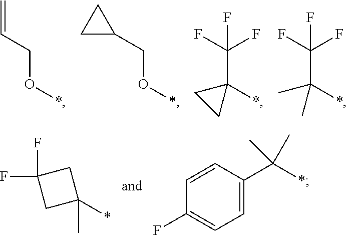

R⁴ is selected from the group R4a, consisting of halo, H, C₁₋₄-alkyl, C₃₋₄-cycloalkyl and F₁₋₉-fluoro-C₁₋₄-alkyl;
or a salt thereof, particularly a pharmaceutically acceptable salt thereof.

Another embodiment of the present invention relates to a compound of formula (I), wherein
A is A2a which is a 5- or 6-membered mono-heteroaryl ring containing one nitrogen or containing two heteroatom members wherein one is nitrogen and the second is selected from nitrogen or sulphur; and wherein A2a is independently substituted with one or two R³;
and substituents R¹, R², R³ and R⁴ are defined as in any of the preceding embodiments.

Another embodiment of the present invention relates to a compound of formula (I), wherein
A is A2b which is a 9- or 10-membered fused bicyclic-heteroaryl ring containing three to four nitrogens; and wherein A2b is independently substituted with one or two R³; and substituents R¹, R², R³ and R⁴ are defined as in any of the preceding embodiments.

Another embodiment of the present invention relates to a compound of formula (I), wherein A is selected from the group A3 consisting of pyridinyl, pyrazinyl, pyrazolyl, isothiazolyl, imidazo[1,2-a]pyrimidyl, pyrazolo[3,4-b]pyridinyl, [1,2,4]triazolo[4,3-a]pyrimidyl, pyrazolo[1,5-b]pyridazinyl, [1,2,4]triazolo[1,5-a]pyrimidinyl, 2H-[1,2,3]triazolo[4,5-b]pyridinyl, 1H-[1,2,3]triazolo[4,5-b]pyridinyl and 1H-imidazo[4,5-b]pyridinyl and wherein A3 is independently substituted with one or two R³;
and substituents R¹, R², R³ and R⁴ are defined as in any of the preceding embodiments.

Another embodiment of the present invention relates to a compound of formula (I), wherein A is selected from the group A4 consisting of pyridinyl, pyrazinyl, pyrazolyl, isothiazolyl, imidazo[1,2-a]pyrimidyl, pyrazolo[3,4-b]pyridinyl, [1,2,4]triazolo[4,3-a]pyrimidyl and pyrazolo[1,5-b]pyridazinyl; and wherein A4 is independently substituted with one or two R³;
and substituents R¹, R², R³ and R⁴ are defined as in any of the preceding embodiments.

Another embodiment of the present invention relates to a compound of formula (I), wherein A is selected from the group A5 consisting of pyridinyl, imidazo[1,2-a]pyrimidyl, pyrazolo[3,4-b]pyridinyl, [1,2,4]triazolo[4,3-a]pyrimidyl and pyrazolo[1,5-b]pyridazinyl; and wherein A5 is independently substituted with one or two R³;
and substituents R¹, R², R³ and R⁴ are defined as in any of the preceding embodiments.

Another embodiment of the present invention relates to a compound of formula (I), wherein A is selected from the group A6 consisting of pyridinyl and pyrazolo[1,5-b]pyridazinyl; and wherein A6 is independently substituted with one or two R³; and substituents R¹, R², R³ and R⁴ are defined as in any of the preceding embodiments.

Another embodiment of the present invention relates to a compound of formula (I), wherein A is selected from the group A7 consisting of pyridinyl; and wherein A7 is independently substituted with one or two R³;
and substituents R¹, R², R³ and R⁴ are defined as in any of the preceding embodiments.

Another embodiment of the present invention relates to a compound of formula (I), wherein A is selected from the group A8 consisting of pyrazolo[1,5-b]pyridazinyl; and wherein A8 is independently substituted with one or two R³;
and substituents R¹, R², R³ and R⁴ are defined as in any of the preceding embodiments.

Another embodiment of the present invention relates to a compound of formula (I), wherein A is selected from the group A9 consisting of

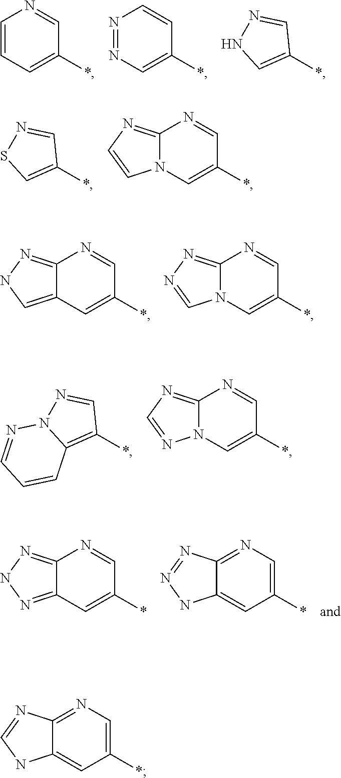

wherein A9 is independently substituted with one or two R³;
and substituents R¹, R², R³ and R⁴ are defined as in any of the preceding embodiments.

Another embodiment of the present invention relates to a compound of formula (I), wherein A is selected from the group A10 consisting of

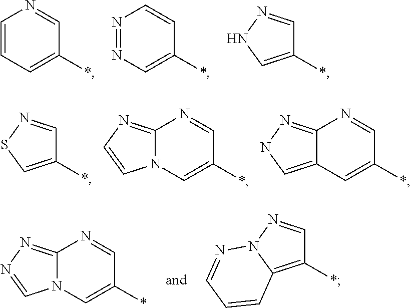

wherein A10 is independently substituted with one or two R³;
and substituents R¹, R², R³ and R⁴ are defined as in any of the preceding embodiments.

Another embodiment of the present invention relates to a compound of formula (I), wherein A is selected from the group A11 consisting of

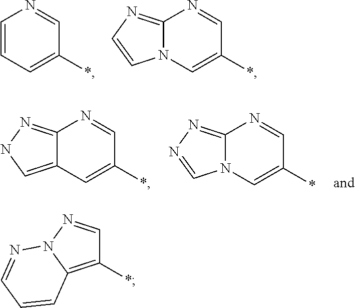

wherein A11 is independently substituted with one or two R³;
and substituents R¹, R², R³ and R⁴ are defined as in any of the preceding embodiments.

Another embodiment of the present invention relates to a compound of formula (I), wherein A is selected from the group A12 consisting of

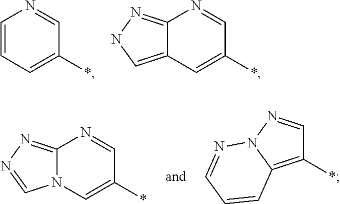

wherein A12 is independently substituted with one or two R³;
and substituents R¹, R², R³ and R⁴ are defined as in any of the preceding embodiments.

Another embodiment of the present invention relates to a compound of formula (I), wherein A is selected from the group A13 consisting of

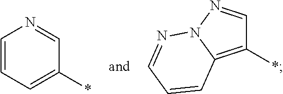

is wherein A13 is independently substituted with one or two R³;
and substituents R¹, R², R³ and R⁴ are defined as in any of the preceding embodiments.

Another embodiment of the present invention relates to a compound of formula (I), wherein A is selected from the group A14 consisting of

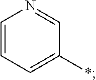

wherein A14 is independently substituted with one or two R³;
and substituents R¹, R², R³ and R⁴ are defined as in any of the preceding embodiments.

Another embodiment of the present invention relates to a compound of formula (I), wherein A is selected from the group A15 consisting of

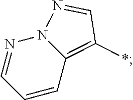

wherein A15 is independently substituted with one or two R³;
and substituents R¹, R², R³ and R⁴ are defined as in any of the preceding embodiments.

Another embodiment of the present invention relates to a compound of formula (I), wherein R¹ is selected from the group Rib, consisting of H, H₃C—, H₃CH₂C—, H₃CH₂CH₂C—, (H₃C)₂HC—, Cl and F;
and substituents A, R², R³ and R⁴ are defined as in any of the preceding embodiments.

Another embodiment of the present invention relates to a compound of formula (I), wherein R¹ is selected from the group R1c, consisting of H, H₃C— and F; and substituents A, R², R³ and R⁴ are defined as in any of the preceding embodiments.

Another embodiment of the present invention relates to a compound of formula (I), wherein R¹ is selected from the group Rid, consisting of H; and substituents A, R², R³ and R⁴ are defined as in any of the preceding embodiments.

Another embodiment of the present invention relates to a compound of formula (I), wherein R¹ is selected from the group R1e, consisting of F;
and substituents A, R², R³ and R⁴ are defined as in any of the preceding embodiments.

Another embodiment of the present invention relates to a compound of formula (I), wherein R¹ is selected from the group Rif, consisting of H₃C— and F;
and substituents A, R², R³ and R⁴ are defined as in any of the preceding embodiments.

Another embodiment of the present invention relates to a compound of formula (I), wherein R² is selected from the group R2b, consisting of H, F, Cl, C₁₋₄-alkyl and F₁₋₉-fluoro-C₁₋₄-alkyl;
and substituents A, R¹, R³ and R⁴ are defined as in any of the preceding embodiments.

Another embodiment of the present invention relates to a compound of formula (I), wherein R² is selected from the group R2c, consisting of H, C₁₋₄-alkyl and F₁₋₉-fluoro-C₁₋₄-alkyl;
and substituents A, R¹, R³ and R⁴ are defined as in any of the preceding embodiments.

Another embodiment of the present invention relates to a compound of formula (I), wherein R² is selected from the group R2d, consisting of H, H₃C—, F₃C—; and substituents A, R¹, R³ and R⁴ are defined as in any of the preceding embodiments.

Another embodiment of the present invention relates to a compound of formula (I), wherein R² is selected from the group R2e, consisting of H;
and substituents A, R¹, R³ and R⁴ are defined as in any of the preceding embodiments.

Another embodiment of the present invention relates to a compound of formula (I), wherein R² is selected from the group R2f, consisting of H₃C— and F₃C—; and substituents A, R¹, R³ and R⁴ are defined as in any of the preceding embodiments.

Another embodiment of the present invention relates to a compound of formula (I), wherein R² is selected from the group R2g, consisting of F, Cl, C₁₋₄-alkyl and F₁₋₉-fluoro-C₁₋₄-alkyl;
and substituents A, R¹, R³ and R⁴ are defined as in any of the preceding embodiments.

Another embodiment of the present invention relates to a compound of formula (I), wherein R³ is selected from the group R3b, consisting of H, halo, C₁₋₄-alkyl, C₃₋₄-cycloalkyl, F₁₋₉-fluoro-C₁₋₄-alkyl, C₁₋₄-alkyloxy, C₃₋₄-cycloalkyloxy and pyrazolyl,

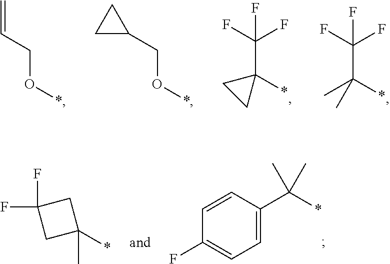

and substituents A, R¹, R² and R⁴ are defined as in any of the preceding embodiments.

Another embodiment of the present invention relates to a compound of formula (I), wherein R³ is selected from the group R3c, consisting of H, F, Cl, H₃C, (H₃C)₃C—, H₃C—O—, F₃C—,

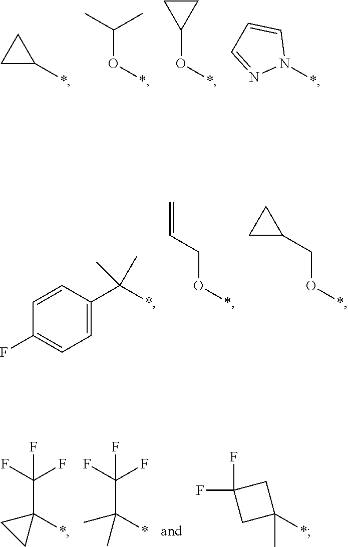

and substituents A, R¹, R² and R⁴ are defined as in any of the preceding embodiments.

Another embodiment of the present invention relates to a compound of formula (I), wherein R³ is selected from the group R3d, consisting of H, F, Cl, H₃C, (H₃C)₃C—, H₃C—O—, F₃C— and

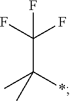

and substituents A, R¹, R² and R⁴ are defined as in any of the preceding embodiments.

Another embodiment of the present invention relates to a compound of formula (I), wherein R³ is selected from the group R3e, consisting of H, H₃C—O—,

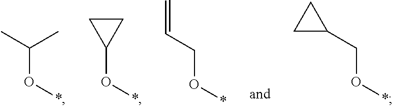

and substituents A, R¹, R² and R⁴ are defined as in any of the preceding embodiments.

Another embodiment of the present invention relates to a compound of formula (I), wherein R³ is selected from the group R3f, consisting of H, H₃C, (H₃C)₃C—, H₃C—O— and F₃C—;
and substituents A, R¹, R² and R⁴ are defined as in any of the preceding embodiments.

Another embodiment of the present invention relates to a compound of formula (I), wherein R³ is selected from the group R3g, consisting of H, F and Cl; and substituents A, R¹, R² and R⁴ are defined as in any of the preceding embodiments.

Another embodiment of the present invention relates to a compound of formula (I), wherein R⁴ is selected from the group R4b, consisting of H, F, Cl, C₁₋₄-alkyl and F₁₋₉-fluoro-C₁₋₄-alkyl;
and substituents A, R¹, R² and R³ are defined as in any of the preceding embodiments.

Another embodiment of the present invention relates to a compound of formula (I), wherein R⁴ is selected from the group R4c, consisting of H, C₁₋₄-alkyl and F₁₋₉-fluoro-C₁₋₄-alkyl;
and substituents A, R¹, R² and R³ are defined as in any of the preceding embodiments.

Another embodiment of the present invention relates to a compound of formula (I), wherein R⁴ is selected from the group R4d, consisting of H, H₃C—, F₃C—; and substituents A, R¹, R² and R³ are defined as in any of the preceding embodiments.

Another embodiment of the present invention relates to a compound of formula (I), wherein R⁴ is selected from the group R4e, consisting of H;
and substituents A, R¹, R² and R³ are defined as in any of the preceding embodiments.

Another embodiment of the present invention relates to a compound of formula (I), wherein R⁴ is selected from the group R4f, consisting of H₃C— and F₃C—; and substituents A, R¹, R² and R³ are defined as in any of the preceding embodiments.

Another embodiment of the present invention relates to a compound of formula (I) above, having formula (I-a)

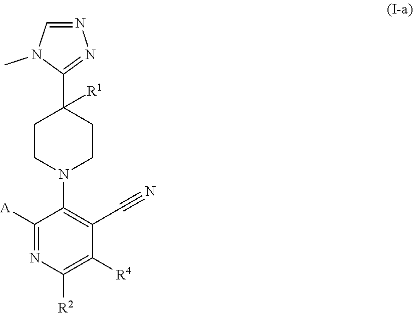

wherein substituents A, R¹, R², R³ and R⁴ are defined as in any of the preceding embodiments.

Another embodiment of the present invention relates to a compound of formula (I) above, having formula (I-b)

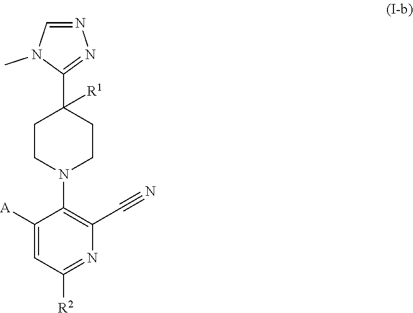

wherein substituents A, R¹, R² and R³ are defined as in any of the preceding embodiments.

Another embodiment of the present invention relates to a compound of formula (I) above, having formula (I-c)

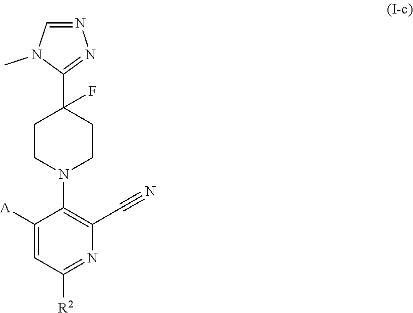

wherein substituents A, R² and R³ are defined as in any of the preceding embodiments.

Another embodiment of the present invention relates to a compound of formula (I) above, having formula (I-d)

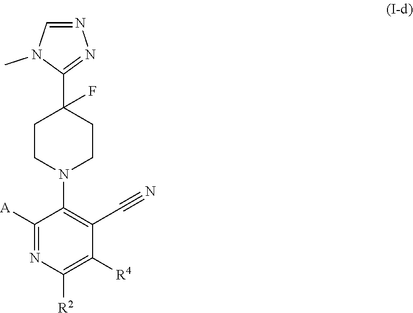

wherein substituents A, R², R³ and R⁴ are defined as in any of the preceding embodiments.

Another embodiment of the present invention relates to a compound of formula (I) above, having formula (I-e)

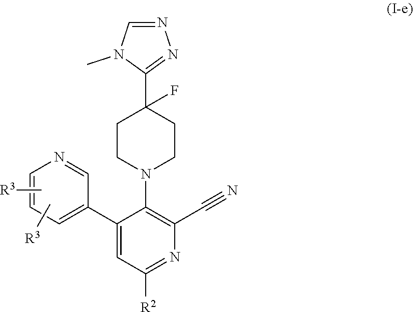

wherein substituents A, R² and R³ are defined as in any of the preceding embodiments.

Another embodiment of the present invention relates to a compound of formula (I) above, having formula (I-f)

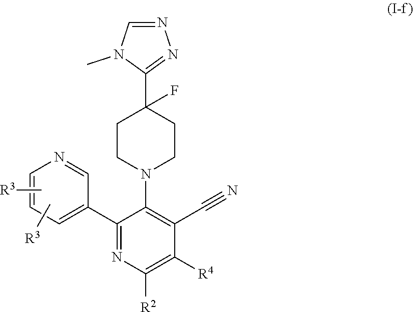

wherein substituents R², R³ and R⁴ are defined as in any of the preceding embodiments.

Further preferred embodiments of the compounds of formula (I) are encompassed as embodiments (EMB-1) to (EMB-20) in the following Table 1, wherein the substituent definitions above are employed.

TABLE 1
further preferred embodiments

| | Substituent | | | | | |
|---|---|---|---|---|---|---|
| Embodiment | formula | A | R¹ | R² | R³ | R⁴ |
| EMB-1 | I | A3 | R1e | R2b | R3b | R4b |
| EMB-2 | I | A9 | R1b | R2b | R3b | R4b |
| EMB-3 | I | A9 | R1e | R2b | R3b | R4b |
| EMB-4 | I | A10 | R1b | R2b | R3b | R4b |
| EMB-5 | I | A10 | R1e | R2b | R3b | R4b |
| EMB-6 | I | A3 | R1b | R2g | R3b | R4b |
| EMB-7 | I | A13 | R1f | R2b | R3b | R4b |
| EMB-8 | I | A13 | R1b | R2b | R3b | R4b |
| EMB-9 | I-a | A3 | R1b | R2b | R3b | R4b |
| EMB-10 | I-a | A14 | R1b | R2b | R3b | R4b |
| EMB-11 | I-b | A3 | R1b | R2b | R3b | — |
| EMB-12 | I-b | A14 | R1b | R2b | R3b | — |
| EMB-13 | I-c | A3 | — | R2b | R3b | — |
| EMB-14 | I-c | A14 | — | R2b | R3b | — |
| EMB-15 | I-d | A3 | — | R2b | R3b | R4b |
| EMB-16 | I-d | A14 | — | R2b | R3b | R4b |
| EMB-17 | I-e | — | — | R2b | R3b | — |
| EMB-18 | I-e | — | — | R2e | R3b | — |
| EMB-19 | I-f | — | — | R2b | R3b | R4b |
| EMB-20 | I-f | — | — | R2b | R3b | R4f |

For example, compounds of the embodiment EMB-1 have for R¹ the genus group R1e as defined above, in combination with the other genus groups for the other substituents in formula (I) as defined within the same row of the table. The same applies analogously to the other variables incorporated in the general formulae.

Particularly preferred is the compound according to formula (I) selected from the group consisting of

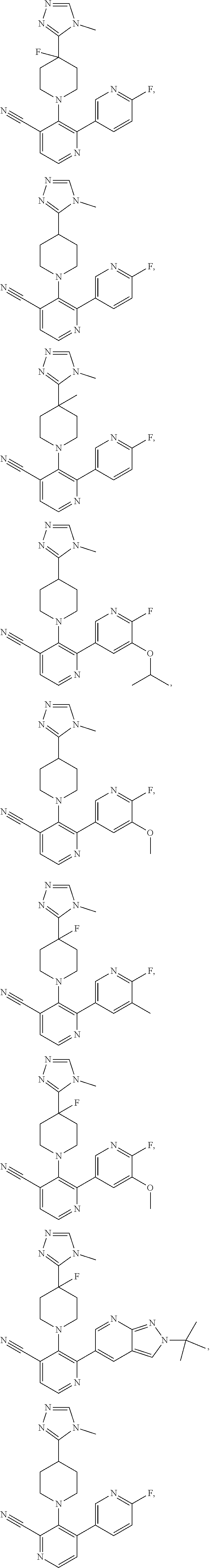

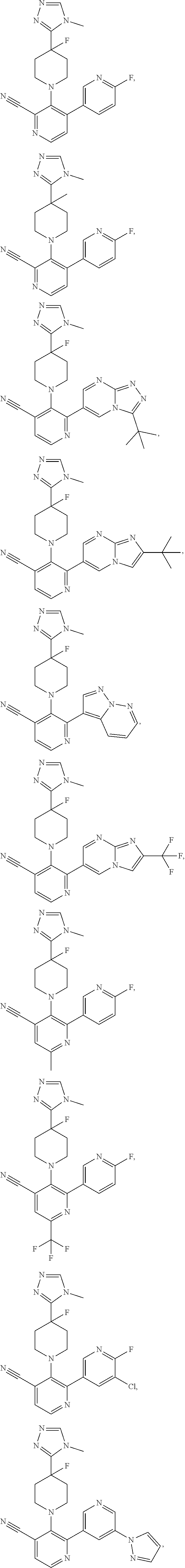

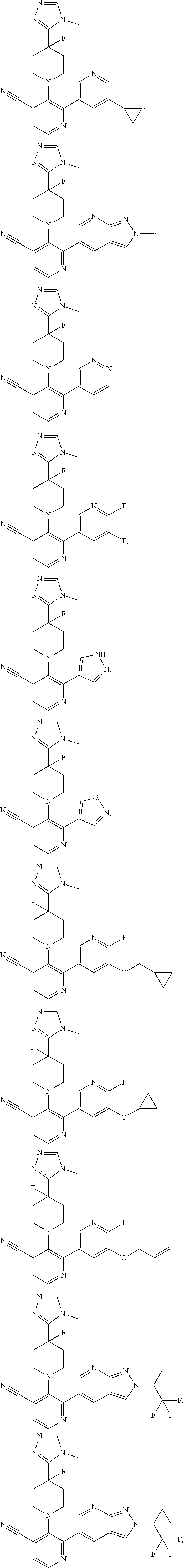

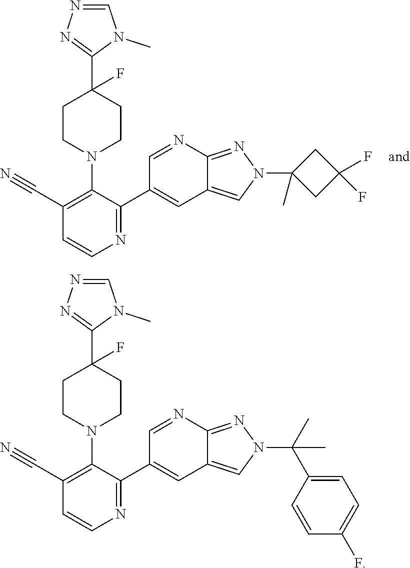

Particularly preferred is the compound according to formula (I) selected from the group consisting of example 1, example 2, example 3, example 4, example 5, example 6, example 7, example 8, example 9, example 10, example 11, example 12, example 13, example 14, example 15, example 16, example 17, example 18, example 19, example 20, example 21, example 22, example 23, example 24, example 25, example 26, example 27, example 28, example 29, example 30, example 31 and example 32, as described hereinafter in EXAMPLES.

Particularly preferred is the compound according to formula (I) selected from the group consisting of example 1, example 3, example 4, example 5, example 6, example 7, example 8, example 12, example 13, example 15, example 16, example 17, example 19, example 21, example 25, example 26, example 27, example 28, example 29, example 30, example 31 and example 32, as described hereinafter in EXAMPLES.

Particularly preferred is the compound according to formula (I) selected from the group consisting of example 1, example 2, example 3, example 4, example 6, example 7, example 10, example 14, example 16, example 17, example 18, example 26 and example 27 as described hereinafter in EXAMPLES.

The present invention provides novel piperidinylpyridinylcarbonitrile derivatives of formula (I) that are surprisingly potent QPCT/L inhibitors.

Another aspect of the invention refers to compounds according to formula (I) as surprisingly having potent inhibition of QPCT/L in cells relevant for, but not limited to, lung diseases or cancer.

Another aspect of the invention refers to compounds according to formula (I) as surprisingly cellular potent QPCT/L inhibitors having appropriate membrane permeability and low in vitro efflux.

Another aspect of the invention refers to pharmaceutical compositions, containing at least one compound according to formula (I) optionally together with one or more inert carriers and/or diluents.

A further aspect of the present invention refers to compounds according to formula (I), for the use in the prevention and/or treatment of disorders associated with QPCT/L inhibition.

Another aspect of the invention refers to processes of manufacture of the compounds of the present invention.

Further aspects of the present invention will become apparent to the skilled artisan directly from the foregoing and following description and the examples.

### USED Terms and Definitions

### General Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined below, the number of carbon atoms is often specified preceding the group, for example, C₁₋₆-alkyl means an alkyl group or radical having 1 to 6 carbon atoms. In general in groups like HO, H₂N, (O)S, (O)₂S, NC (cyano), HOOC, F₃C or the like, the skilled artisan can see the radical attachment point(s) to the molecule from the free valences of the group itself. For combined groups comprising two or more subgroups, the last named subgroup is the radical attachment point, for example, the substituent “aryl-C₁₋₃-alkylene” means an aryl group which is bound to a C₁₋₃-alkyl-group, the latter of which is bound to the core or to the group to which the substituent is attached.

In case a compound of the present invention is depicted in the form of a chemical name and as a formula, in case of any discrepancy the formula shall prevail. An asterisk may be used in sub-formulas to indicate the bond which is connected to the core molecule as defined.

The numeration of the atoms of a substituent starts with the atom which is closest to the core or to the group to which the substituent is attached.

For example, the term “3-carboxypropyl-group” represents the following substituent:

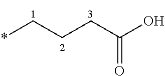 [SMILES: *CCCC(=O)O]

wherein the carboxy group is attached to the third carbon atom of the propyl group. The terms “1-methylpropyl-”, “2,2-dimethylpropyl-” or “cyclopropylmethyl-” group represent the following groups:

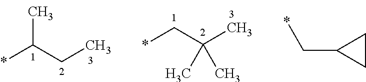

The asterisk may be used in sub-formulas to indicate the bond which is connected to the core molecule as defined.

The term “substituted” as used herein, means that one or more hydrogens on the designated atom are replaced by a group selected from a defined group of substituents, provided that the designated atom's normal valence is not exceeded, and that the substitution results in a stable compound. Likewise, the term “substituted” may be used in connection with a chemical moiety instead of a single atom, e.g. “substituted alkyl”, “substituted aryl” or the like.

Unless specifically indicated, throughout the specification and the appended claims, a given chemical formula or name shall encompass tautomers and all stereo, optical and geometrical isomers (e.g. enantiomers, diastereomers, E/Z isomers etc. . . . ) and racemates thereof as well as mixtures in different proportions of the separate enantiomers, mixtures of diastereomers, or mixtures of any of the foregoing forms where such isomers and enantiomers exist, as well as solvates thereof such as for instance hydrates.

Unless specifically indicated, also “pharmaceutically acceptable salts” as defined in more detail below shall encompass solvates thereof such as for instance hydrates.

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents.

Enantiomerically pure compounds of this invention or intermediates may be prepared via asymmetric synthesis, for example by preparation and subsequent separation of appropriate diastereomeric compounds or intermediates which can be separated by known methods (e.g. by chromatographic separation or crystallization) and/or by using chiral reagents, such as chiral starting materials, chiral catalysts or chiral auxiliaries.

Further, it is known to the person skilled in the art how to prepare enantiomerically pure compounds from the corresponding racemic mixtures, such as by chromatographic separation of the corresponding racemic mixtures on chiral stationary phases; or by resolution of a racemic mixture using an appropriate resolving agent, e.g. by means of diastereomeric salt formation of the racemic compound with optically active acids or bases, subsequent resolution of the salts and release of the desired compound from the salt; or by derivatization of the corresponding racemic compounds with optically active chiral auxiliary reagents, subsequent diastereomer separation and removal of the chiral auxiliary group; or by kinetic resolution of a racemate (e.g. by enzymatic resolution); by enantioselective crystallization from a conglomerate of enantiomorphous crystals under suitable conditions; or by (fractional) crystallization from a suitable solvent in the presence of an optically active chiral auxiliary.

The phrase “pharmaceutically acceptable” is employed herein to refer to those compounds, materials, compositions, and/or dosage forms which are, within the scope of sound medical judgment, suitable for use in contact with the tissues of human beings without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent such as ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts,) also comprise a part of the invention.

The term halogen denotes fluorine, chlorine, bromine and iodine.

The term “C₁₋ₙ-alkyl”, wherein n is an integer selected from 2, 3, 4, 5 or 6, preferably 4, 5, or 6, either alone or in combination with another radical, denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

The term “C₃₋ₖcycloalkyl”, wherein k is an integer selected from 3, 4, 5, 7 or 8, preferably 4, 5 or 6, either alone or in combination with another radical, denotes a cyclic, saturated, unbranched hydrocarbon radical with 3 to k C atoms. For example the term C₃₋₇-cycloalkyl includes cyclopropyl, cyclobutyl, cyclopentyl, cyclohexyl and cycloheptyl.

The term “halo” added to an “alkyl”, “alkylene” or “cycloalkyl” group (saturated or unsaturated) defines an alkyl, alkylene or cycloalkyl group wherein one or more hydrogen atoms are replaced by a halogen atom selected from among fluorine, chlorine or bromine, preferably fluorine and chlorine, particularly preferred is fluorine. Examples include: H₂FC—, HF₂C—, F₃C—.

The term “mono-heteroaryl ring” means a monocyclic aromatic ring system, containing one or more heteroatoms selected from N or S, consisting of 5 to 6 ring atoms.

The term “mono-heteroaryl ring” is intended to include all the possible isomeric forms. Thus, the term “mono-heteroaryl ring” includes the following exemplary structures (not depicted as radicals as each form is optionally attached through a covalent bond to any atom so long as appropriate valences are maintained):

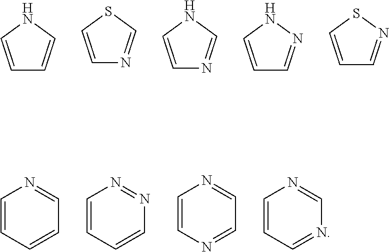

The term “fused bicyclic-heteroaryl ring” means a bicyclic aromatic ring system, containing one or more heteroatoms selected from N or S, consisting of 9 to 10 ring atoms. The term “fused bicyclic-heteroaryl ring” is intended to include all the possible isomeric forms. Thus, the term “bicyclic-heteroaryl ring” includes the following exemplary structures (not depicted as radicals as each form is optionally attached through a covalent bond to any atom so long as appropriate valences are maintained):

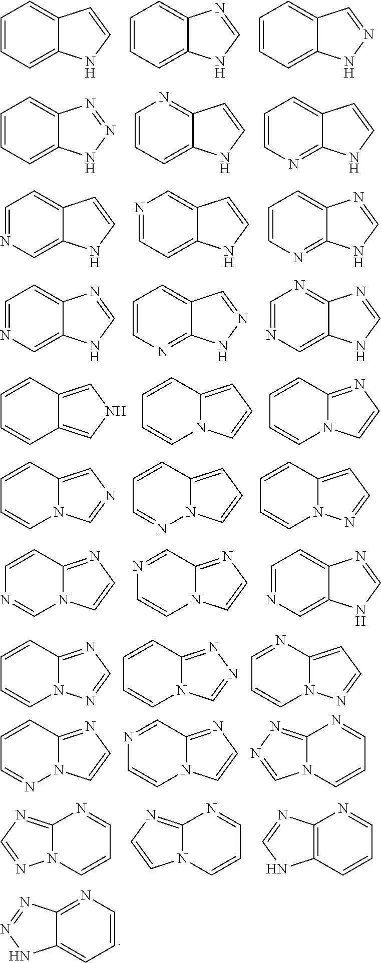

The term pyridinyl refers to the radical of the following ring:

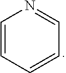

The term pyrazinyl refers to the radical of the following ring:

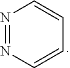

The term pyrazolyl refers to the radical of the following ring:

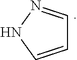

The term isothiazolyl refers to the radical of the following ring:

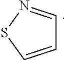

The term imidazo[1,2-a]pyrimidyl refers to the radical of the following ring:

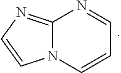

The term pyrazolo[3,4-b]pyridinyl refers to the radical of the following ring:

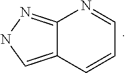

The term [1,2,4]triazolo[4,3-a]pyrimidyl refers to the radical of the following ring:

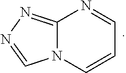

The term pyrazolo[1,5-b]pyridazinyl refers to the radical of the following ring:

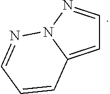

The term [1,2,4]triazolo[1,5-a]pyrimidinyl refers to the radical of the following ring:

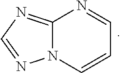

The term 2H-[1,2,3]triazolo[4,5-b]pyridinyl refers to the radical of the following ring:

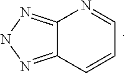

The term 1H-[1,2,3]triazolo[4,5-b]pyridinyl refers to the radical of the following ring:

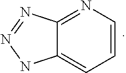

The term 1H-imidazo[4,5-b]pyridinyl refers to the radical of the following ring:

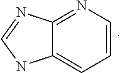

Many of the terms given above may be used repeatedly in the definition of a formula or group and in each case have one of the meanings given above, independently of one another.

### BIOLOGICAL ASSAYS

### Evaluation of Inhibitory Activity on QPCT and QPCTL

### Assay A: Biochemical QPCT and QPCTL Activity Assay

The activity of the compounds of the invention may be demonstrated using the following biochemical enzyme activity assay:

QPCT or QPCTL dependent conversion of N-terminal glutamine to pyroglutamate of CD47 was monitored via MALDI-TOF MS. Test compounds were dissolved in 100% DMSO and serially diluted into clear 1,536-well microtiter plates. Enzymatic reactions were set up in assay buffer containing 20 mM Tris pH 7.5, 0.1 mM TCEP, 0.01% BSA, and 0.001% Tween20. 2.5 μL of 2× concentrated QPCTL (in-house) or QPCT (Origine #TP700028) enzyme in assay buffer (0.5 nM final concentration, columns 1-23) or plain assay buffer (columns 24) were added to each well. The plates were incubated for 10 min in a humidified incubator at 24° C. Subsequently, 2.5 μL of CD47 peptide substrate surrogate (¹⁹QLLFNKTKSVEFTFC³³) was added to each well (final concentration: 10 μM for QPCTL/20 μM for QPCT). The plates were mixed for 30 sec at 1,000 rpm and subsequently incubated for 40 min in a humidified incubator at 24° C. After incubation, the enzymatic reaction was stopped by adding 1 μL containing stable isotope labeled internal standard peptide ¹⁹[Pyr]LLFN(K)TKSVEFTFC³³ (final concentration 4.0 μM) as well as SEN177 (final concentration 10 μM). The plates were sealed with an adhesive foil, mixed for 30 s at 1,000 rpm and stored at room temperature until preparation of the MALDI target plates. MALDI target plates were prepared as described previously.1 Mass spectra were acquired with a rapifleX MALDI-TOF/TOF instrument tracking the signals of the product (¹⁹[Pyr]LLFNKTKSVEFTFC³³, m/z 1,787.9037) as well as internal standard (¹⁹[Pyr]LLFN(K)TKSVEFTFC³³, m/z 1,795.9179) peptide. QPCT or QPCTL activity was monitored by calculating the ratio between product and internal standard signals followed by normalization to high (100% activity) and low (0% activity) controls. Determination of compound potencies was obtained by fitting the dose-response data to a four-parameter logistical equation.

TABLE 2
Biological data for compounds of the invention as obtained in Assay A.

| | Inhibition of | Inhibition of |
|---|---|---|
| | QPCTL: | QPCT: |
| Example | IC₅₀ [nM] | IC₅₀ [nM] |
| 1 | 2 | 4 |
| 2 | 2 | 17 |
| 3 | 2 | 2 |
| 4 | 1 | 12 |
| 5 | 2 | 13 |
| 6 | 2 | 8 |
| 7 | 2 | 12 |
| 8 | <1 | 1 |
| 9 | 4 | 26 |
| 10 | 4 | 8 |
| 11 | 14 | 11 |
| 12 | 2 | 2 |
| 13 | 1 | 1 |
| 14 | 11 | 10 |
| 15 | 1 | 1 |
| 16 | 1 | 4 |
| 17 | 4 | 8 |
| 18 | 8 | 23 |
| 19 | 2 | 10 |
| 20 | 21 | 29 |
| 21 | 1 | 3 |
| 22 | 3 | 12 |
| 23 | 6 | 32 |
| 24 | 18 | 31 |
| 25 | 10 | 30 |
| 26 | 1 | 1 |
| 27 | 3 | 5 |
| 28 | 2 | 3 |
| 29 | 2 | 3 |
| 30 | 2 | 2 |
| 31 | 2 | 3 |
| 32 | 1 | 1 |

TABLE 3
Biological data for prior art compounds as obtained in Assay A.

| | | Inhibition of | Inhibition of |
|---|---|---|---|
| Prior art | | QPCTL: | QPCT: |
| Compound | Reference | IC₅₀ [nM] | IC₅₀ [nM] |
| SEN177 | J-S, NCB 2015; P, | 17 | 79 |
| | JBIC 2018 | | |
| SEN180 | J-S, NCB 2015 | 39 | 176 |
| 1094 | WO 2018/178384 | 111 | 1707 |
| 1095 | WO 2018/178384 | 13 | 260 |

### Assay B: SIRPα Signalling Assay (Using Either Raji or A549 Cells)

The activity of the compounds of the invention may be demonstrated using the following SIRPα signalling assay that measures SIRPα engagement induced by CD47 presented via cell-cell interaction. Two cell types are independently used: the Raji cell line (lymphoblast-like human cell line derived from B lymphocytes from a Burkitt's lymphoma patient in 1963) and A549 cells (adenocarcinomic human alveolar basal epithelial cells).

Test compounds were dissolved in 100% DMSO and serially diluted into a white 384-well microtiter cell culture plate (PerkinElmer #60076780 in case of Raji assay; PDL-coated plates Greiner #781945 in case of A549 assay). 5000 Raji cells (ATCC #CC86) or 5000 A549 cells (ATCC #CCL-185) in Assay Complete Cell Plating reagent 30 (DiscoverX 93-0563R30B) were added per well. The assay plate was incubated for 48 h at 37° C., 95% humidity and 5% CO₂. 15000 reporter cells (Jurkat PathHunter SIRPαV1, DiscoverX #93-1135C19) were added to each well, and the plate was incubated for 5 h at 37° C., 95% humidity and 5% CO₂. Bioassay reagent 1 of the PathHunter Bioassay detection kit (DiscoverX 93-0001) was added to each well of the plate using a multichannel pipette followed by a 15 min incubation at room temperature. Afterwards bioassay reagent 2 was added followed by 60 min incubation at room temperature (incubation in the dark).

The analysis of the data was performed using the luminescence signal generated by beta-galactosidase in the PathHunter reporter cell line. The luminescence measurement was done using a Pherastar Multi-Mode Reader. Dose-response curves & IC₅₀ data were calculated with 4-parameter sigmoidal dose response formula.

TABLE 4
Biological data for compounds of the invention as obtained in Assay B.

| | Inhibition of | Inhibition of |
|---|---|---|
| | SIRPα signalling | SIRPα signalling |
| | induced by Raji | induced by A549 |
| | cells: | cells: |
| Example | IC₅₀ [nM] | IC₅₀ [nM] |
| 1 | 168 | 51 |
| 2 | 245 | 60 |
| 3 | 467 | 29 |
| 4 | 182 | 38 |
| 5 | 350 | 44 |
| 6 | 190 | 34 |
| 7 | 214 | 27 |
| 8 | 25 | 22 |
| 9 | 1092 | |
| 10 | 352 | |
| 11 | 1818 | 237 |
| 12 | 53 | 31 |
| 13 | 65 | 22 |
| 14 | 436 | 116 |
| 15 | 59 | 33 |
| 16 | 86 | 8 |
| 17 | 92 | 49 |
| 18 | 482 | 112 |
| 19 | 509 | 18 |
| 20 | 1951 | 342 |
| 21 | 322 | 22 |
| 22 | 653 | 103 |
| 23 | 1665 | 114 |
| 24 | 1766 | 693 |
| 25 | 1608 | 44 |
| 26 | 103 | 7 |
| 27 | 176 | 39 |
| 28 | 180 | 33 |
| 29 | 81 | 13 |
| 30 | 87 | 47 |
| 31 | 179 | 21 |
| 32 | 6 | 3 |

TABLE 5
Biological data for prior art compounds as obtained in Assay B.

| | | Inhibition of | Inhibition of |
|---|---|---|---|
| | | SIRPα signalling | SIRPα signalling |
| | | induced by Raji | induced by A549 |
| Prior art | | cells: | cells: |
| Compound | Reference | IC₅₀ [nM] | IC₅₀ [nM] |
| SEN177 | J-S, NCB 2015; P, | 1365 | 214 |
| | JBIC 2018 | | |
| SEN180 | J-S, NCB 2015 | 2973 | 868 |
| 1094 | WO 2018/178384 | 3240 | |
| 1095 | WO 2018/178384 | 2203 | 161 |

### Evaluation of Permeability

### Assay C: Permeability in CACO-2 Cells

Caco-2 cells (1-2×105 cells/1 cm2 area) are seeded on filter inserts (Costar transwell polycarbonate or PET filters, 0.4 μm pore size) and cultured (DMEM) for 10 to 25 days. Compounds are dissolved in appropriate solvent (like DMSO, 1-20 mM stock solutions). Stock solutions are diluted with HTP-4 buffer (128.13 mM NaCl, 5.36 mM KCl, 1 mM MgSO₄, 1.8 mM CaCl₂, 4.17 mM NaHCO₃, 1.19 mM Na₂HPO₄×7H₂0, 0.41 mM NaH₂PO₄×H₂O, 15 mM HEPES, 20 mM glucose, 0.25% BSA, pH 7.2) to prepare the transport solutions (0.1-300 μM compound, final DMSO <=0.5%). The transport solution (TL) is applied to the apical or basolateral donor side for measuring A-B or B-A permeability (3 filter replicates), respectively. Samples are collected at the start and end of experiment from the donor and at various time intervals for up to 2 hours also from the receiver side for concentration measurement by HPLC-MS/MS or scintillation counting. Sampled receiver volumes are replaced with fresh receiver solution.

Efflux ratio (ER)=permeability B−A/permeability A−B

TABLE 7
Biological data for compounds of the invention as obtained in Assay C.

| | Permeability A-B | |
|---|---|---|
| Example | [10⁻⁶ cm/s] | Efflux Ratio |
| 1 | 28.0 | 2.3 |
| 2 | 4.8 | 3.5 |
| 3 | 4.9 | 8.4 |
| 4 | 8.5 | 5.9 |
| 5 | 2.0 | 14.0 |
| 6 | 34.0 | 2.3 |
| 7 | 18.0 | 3.3 |
| 8 | 1.3 | 30.0 |
| 9 | 1.8 | 7.8 |
| 10 | 11.0 | 5.6 |
| 11 | 3.1 | 11.6 |
| 12 | 2.5 | 11.2 |
| 13 | 1.0 | 35.0 |
| 14 | 41.0 | 0.6 |
| 15 | 0.9 | 14.1 |
| 16 | 40.0 | 1.7 |
| 17 | 38.0 | 1.3 |
| 18 | 41.0 | 1.4 |
| 19 | 4.9 | 6.5 |
| 20 | | |
| 21 | | |
| 22 | 0.2 | 10.0 |
| 23 | | |
| 24 | | |
| 25 | | |
| 26 | 45.0 | 1.7 |
| 27 | 36.0 | 2.0 |
| 28 | | |
| 29 | 2.5 | 15.2 |
| 30 | 0.5 | 54.9 |
| 31 | 1.7 | 27.6 |
| 32 | 3.5 | 12.6 |

TABLE 8
Biological data for prior art compounds as obtained in Assay C.

| Prior art | | Permeability A-B | Efflux |
|---|---|---|---|
| Compound | Reference | [10⁻⁶ cm/s] | Ratio |
| SEN177 | J-S, NCB 2015; | 11.0 | 3.3 |
| | P, JBIC 2018 | | |
| SEN180 | J-S, NCB 2015 | 4.2 | 7.1 |
| 1094 | WO 2018/178384 | 73 | 1.0 |
| 1095 | WO 2018/178384 | 68 | 0.5 |

### Evaluation of Microsomal Clearance

### Microsomal Clearance:

The metabolic degradation of the test compound was assayed at 37° C. with pooled liver microsomes from various species. The final incubation volume of 60 μl per time point contains TRIS buffer pH 7.6 at room temperature (0.1 M), magnesium chloride (5 mM), microsomal protein (1 mg/mL for human and dog, 0.5 mg/mL for other species) and the test compound at a final concentration of 1 μM. Following a short preincubation period at 37° C., the reactions were initiated by addition of betanicotinamide adenine dinucleotide phosphate, reduced form (NADPH, 1 mM), and terminated by transferring an aliquot into solvent after different time points. After centrifugation (10000 g, 5 min), an aliquot of the supernatant was assayed by LC-MS/MS for the amount of parent compound. The half-life was determined by the slope of the semi-logarithmic plot of the concentration-time profile.

The intrinsic clearance (CL_INTRINSIC) is calculated by considering the amount of protein in the incubation:

CL_INTRINSIC [μl/min/mg protein]=(Ln 2/(half-life [min]*protein content [mg/ml]))*1000

CL_INTRINSIC_INVIVO [ml/min/kg]=(CL_INTRINSIC [μL/min/mg protein]×MPPGL [mg protein/g liver]×liver factor [g/kg bodyweight])/1000

Qh [%]=CL [ml/min/kg]/hepatic blood flow [ml/min/kg])

Hepatocellularity, human: 120×10e6 cells/g liver

Liver factor, human: 25.7 g/kg bodyweight

Blood flow, human: 21 ml/(min×kg)

### Evaluation of Hepatocyte Clearance

### Hepatocyte Clearance

The metabolic degradation of a test compound is assayed in a human hepatocyte suspension. After recovery from cryopreservation, human hepatocytes are diluted in Dulbecco's modified eagle medium (supplemented with 3.5 μg glucagon/500 mL, 2.5 mg insulin/500 mL, 3.75 mg hydrocortisone/500 mL, 5% human serum) to obtain a final cell density of 1.0×10⁶ cells/mL.

Following a 30 minutes preincubation in a cell culture incubator (37° C., 10% CO₂), test compound solution is spiked into the hepatocyte suspension, resulting in a final test compound concentration of 1 μM and a final DMSO concentration of 0.05%.

The cell suspension is incubated at 37° C. (cell culture incubator, horizontal shaker) and samples are removed from the incubation after 0, 0.5, 1, 2, 4 and 6 hours. Samples are quenched with acetonitrile (containing internal standard) and pelleted by centrifugation. The supernatant is transferred to a 96-deepwell plate, and prepared for analysis of decline of parent compound by HPLC-MS/MS.

The percentage of remaining test compound is calculated using the peak area ratio (test compound/internal standard) of each incubation time point relative to the time point 0 peak area ratio. The log-transformed data are plotted versus incubation time, and the absolute value of the slope obtained by linear regression analysis is used to estimate in vitro half-life (T½).

In vitro intrinsic clearance (CLint) is calculated from in vitro T½ and scaled to whole liver using a hepatocellularity of 120×106 cells/g liver, a human liver per body weight of 25.7 g liver/kg as well as in vitro incubation parameters, applying the following equation:

CL_INTRINSIC_IN VIVO [mL/min/kg]=(CL_INTRINSIC [μL/min/106 cells]×hepatocellularity [106 cells/g liver]×liver factor [g/kg body weight])/1000

Hepatic in vivo blood clearance (CL) is predicted according to the well-stirred liver model considering an average liver blood flow (QH) of 20.7 mL/min/kg:

CL [mL/min/kg]=CL_INTRINSIC_IN VIVO [mL/min/kg]×hepatic blood flow [mL/min/kg]/(CL_INTRINSIC_IN VIVO [mL/min/kg]+hepatic blood flow [mL/min/kg])

Results are expressed as percentage of hepatic blood flow:

QH [%]=CL [mL/min/kg]/hepatic blood flow [mL/min/kg])

### Evaluation of Plasma Protein Binding

Equilibrium dialysis technique is used to determine the approximate in vitro fractional binding of test compounds to plasma proteins applying Dianorm Teflon dialysis cells (micro 0.2). Each dialysis cell consists of a donor and an acceptor chamber, separated by an ultrathin semipermeable membrane with a 5 kDa molecular weight cutoff. Stock solutions for each test compound are prepared in DMSO at 1 mM and serially diluted to obtain a final test concentration of 1 μM. The subsequent dialysis solutions are prepared in plasma (supplemented with NaEDTA as anticoagulant), and aliquots of 200 μl test compound dialysis solution in plasma are dispensed into the donor (plasma) chambers. Aliquots of 200 μl dialysis buffer (100 mM potassium phosphate, pH 7.4, supplemented with up to 4.7% Dextran) are dispensed into the buffer (acceptor) chamber. Incubation is carried out for 2 hours under rotation at 37° C. for establishing equilibrium.

At the end of the dialysis period, aliquots obtained from donor and acceptor chambers, respectively, are transferred into reaction tubes and processed for HPLC-MS/MS analysis. Analyte concentrations are quantified in aliquots of samples by HPLC-MS/MS against calibration curves.

Percent bound is calculated using the formula:

% bound=(plasma concentration−buffer concentration/plasma concentration)×100

### Evaluation of Solubility

Saturated solutions are prepared in well plates (format depends on robot) by adding an appropriate volume of selected aqueous media (typically in the range of 0.25-1.5 ml) into each well which contains a known quantity of solid drug substance (typically in the range 0.5-5.0 mg). The wells are shaken or stirred for a predefined time period (typically in a range of 2-24 h) and then filtered using appropriate filter membranes (typically PTFE-filters with 0.45 μm pore size). Filter absorption is avoided by discarding the first few drops of filtrate. The amount of dissolved drug substance is determined by UV spectroscopy. In addition, the pH of the aqueous saturated solution is measured using a glass-electrode pH meter.

### Evaluation of Metabolism in Human Hepatocytes In Vitro

The metabolic pathway of a test compound is investigated using primary human hepatocytes in suspension. After recovery from cryopreservation, human hepatocytes are incubated in Dulbecco's modified eagle medium containing 5% human serum and supplemented with 3.5 μg glucagon/500 ml, 2.5 mg insulin/500 ml and 3.75 mg/500 ml hydrocortisone.

Following a 30 min preincubation in a cell culture incubator (37° C., 10% CO₂), test compound solution is spiked into the hepatocyte suspension to obtain a final cell density of 1.0*10⁶ to 4.0*10⁶ cells/ml (depending on the metabolic turnover rate of the compound observed with primary human hepatocytes), a final test compound concentration of 10 μM, and a final DMSO concentration of 0.05%.

The cells are incubated for six hours in a cell culture incubator on a horizontal shaker, and samples are removed from the incubation after 0, 0.5, 1, 2, 4 or 6 hours, depending on the metabolic turnover rate. Samples are quenched with acetonitrile and pelleted by centrifugation. The supernatant is transferred to a 96-deepwell plate, evaporated under nitrogen and resuspended prior to bioanalysis by liquid chromatography-high resolution mass spectrometry for identification of putative metabolites.

The structures are assigned tentatively based on Fourier-Transform-MSⁿ data. Metabolites are reported as percentage of the parent in human hepatocyte incubation with a threshold of ≥4%.

### Evaluation of Pharmacokinetic Characteristics

The test compound is administered either intravenously or orally to the respective test species. Blood samples are taken at several time points post application of the test compound, anticoagulated and centrifuged.

The concentration of analytes—the administered compound and/or metabolites—are quantified in the plasma samples. PK parameters are calculated using non compartment methods. AUC and Cmax are normalized to a dose of 1 μmol/kg.

### Method of Treatment

The present invention is directed to compounds of general formula (I) which are useful in the prevention and/or treatment of a disease and/or condition associated with or modulated by QPCT/L activity, including but not limited to the treatment and/or prevention of cancer, fibrotic diseases, neurodegenerative diseases, atherosclerosis, infectious diseases, chronic kidney diseases.

The compounds of general formula (I) are useful for the prevention and/or treatment of:
(1) Pulmonary fibrotic diseases such as pneumonitis or interstitial pneumonitis associated with collagenosis, e g. lupus erythematodes, systemic scleroderma, rheumatoid arthritis, polymyositis and dermatomysitis, idiopathic interstitial pneumonias, such as pulmonary lung fibrosis (IPF), non-specific interstitial pneumonia, respiratory bronchiolitis associated interstitial lung disease, desquamative interstitial pneumonia, cryptogenic orgainizing pneumonia, acute interstitial pneumonia and lymphocytic interstitial pneumonia, lymangioleiomyomatosis, pulmonary alveolar proteinosis, Langerhan's cell histiocytosis, pleural parenchymal fibroelastosis, interstitial lung diseases of known cause, such as interstitial pneumonitis as a result of occupational exposures such as asbestosis, silicosis, miners lung (coal dust), farmers lung (hay and mould), Pidgeon fanciers lung (birds) or other occupational airbourne triggers such as metal dust or mycobacteria, or as a result of treatment such as radiation, methotrexate, amiodarone, nitrofurantoin or chemotherapeutics, or for granulomatous disease, such as granulomatosis with polyangitis, Churg-Strauss syndrome, sarcoidosis, hypersensitivity pneumonitis, or interstitial pneumonitis caused by different origins, e g. aspiration, inhalation of toxic gases, vapors, bronchitis or pneumonitis or interstitial pneumonitis caused by heart failure, X-rays, radiation, chemotherapy, M. boeck or sarcoidosis, granulomatosis, cystic fibrosis or mucoviscidosis, or alpha-1-antitrypsin deficiency.
(2) Other fibrotic diseases such as hepatic bridging fibrosis, liver cirrhosis, non-alcoholic steatohepatitis (NASH), atrial fibrosis, endomyocardial fibrosis, old myocardial infarction, glial scar, arterial stiffness, arthrofibrosis, Dupuytren's contracture, keloid, scleroderma/systemic sclerosis, mediastinal fibrosis, myelotibrosis, Peyronie's disease, nephrogenic systemic fibrosis, retroperitoneal fibrosis, adhesive capsulitis; spontaneous acute exacerbations in pulmonary fibrosis and progressive pulmonary fibrosis or induced by infection, microaspiration, surgical lung biopsy, surgical resection, bronchoscopy (BAL, cryobiopsy), air pollution, prior exacerbation and medications.
(3) Leukemia, acute myeloid leukemia (AML), acute promyelocytic leukemia (APL), chronic myeloid leukemia (CML), acute lymphoblastic leukemia (ALL), T-cell acute lymphoblastic leukemia (T-ALL), lymphoma, B-cell lymphoma, T-cell lymphoma, Hodgkin's disease, non-Hodgkin's lymphoma (NHL), hairy cell lymphoma, Burkett's lymphoma, multiple myeloma (MM), myelodysplastic syndrome, solid cancer, lung cancer, adenocarcinoma, small cell lung cancer (SCLC), non-small cell lung cancer (NSCLC), mediastinum cancer, peritoneal cancer, mesothelioma, gastrointestinal cancer, gastric cancer, stomach cancer, bowel cancer, small bowel cancer, large bowel cancer, colon cancer, colon adenocarcinoma, colon adenoma, rectal cancer, colorectal cancer, leiomyosarcoma, breast cancer, gynaecological cancer, genito-urinary cancer, ovarian cancer, endometrial cancer, cervical cancer, prostate cancer, testicular cancer, seminoma, teratocarcinoma, liver cancer, kidney cancer, bladder cancer, urothelial cancer, biliary tract cancer, pancreatic cancer, exocrine pancreatic carcinoma, esophageal cancer, nasopharyngeal cancer, head and neck squamous cell carcinoma (HNSCC), skin cancer, squamous cancer, squamous cell carcinoma, Kaposi's sarcoma, melanoma, malignant melanoma, xeroderma pigmentosum, keratoacanthoma, bone cancer, bone sarcoma, osteosarcoma, rhabdomyosarcoma, fibrosarcoma, thyroid gland cancer, thyroid follicular cancer, adrenal gland cancer, nervous system cancer, brain cancer, astrocytoma, neuroblastoma, glioma, schwannoma, glioblastoma, or sarcoma, gastrointestinal cancer, gastric cancer, stomach cancer, esophageal cancer, head and neck squamous cell carcinoma (HNSCC), breast cancer, colorectal cancer, bowel cancer, large bowel cancer, colon cancer, colon adenocarcinoma, colon adenoma, rectal cancer, ovarian cancer, pancreatic cancer, exocrine pancreatic carcinoma, leukemia, acute myeloid leukemia (AML), myelodysplastic syndrome, lymphoma, B-cell lymphoma, non-Hodgkin's lymphoma (NHL), urothelial cancer, or peritoneal cancer.
(4) Inflammatory, auto-immune or allergic diseases and conditions such as asthma, pediatric asthma, allergic bronchitis, alveolitis, hyperreactive airways, allergic conjunctivitis, bronchiectasis, adult respiratory distress syndrome, bronchial and pulmonary edema, bronchitis or pneumonitis, non-allergic asthma, chronic obstructive pulmonary disease (COPD), acute bronchitis, chronic bronchitis, pulmonary emphysema; autoimmune diseases, such as rheumatoid arthritis, Graves' disease, Sjogren's syndrome psoriatic arthritis, multiple sclerosis, systemic lupus Erythematosus, inflammatory bowel diseases, such as Crohn's disease and ulcerative colitis, scleroderma; psoriasis (including T-cell mediated psoriasis) and inflammatory dermatoses such as an dermatitis, eczema, atopic dermatitis, allergic contact dermatitis, urticaria; vasculitis (e g, necrotizing, cutaneous, and hypersensitivity vasculitis), or erythemanodosum.
(5) Neurodegenerative disorders such as amyotrophic lateral sclerosis, multiple sclerosis, Parkinson's disease, Alzheimer's disease, Huntington's disease, multiple system atrophy, or prion diseases.

Accordingly, the present invention relates to a compound of general formula (I) or a pharmaceutically acceptable salt thereof for use as a medicament.

Furthermore, the present invention relates to the use of a compound of general formula (I) for the treatment and/or prevention of a disease and/or condition associated with or modulated by QPCT/L activity.

Furthermore, the present invention relates to the use of a compound of general formula (I) or a pharmaceutically acceptable salt thereof or a pharmaceutical composition thereof for the treatment and/or prevention of cancer, fibrotic diseases, neurodegenerative diseases, atherosclerosis, infectious diseases, chronic kidney diseases.

Furthermore, the present invention relates to the use of a compound of general formula (I) or a pharmaceutically acceptable salt thereof or a pharmaceutical composition thereof for the treatment and/or prevention of: (1) Pulmonary fibrotic diseases such as pneumonitis or interstitial pneumonitis associated with collagenosis, e g. lupus erythematodes, systemic scleroderma, rheumatoid arthritis, polymyositis and dermatomysitis, idiopathic interstitial pneumonias, such as pulmonary lung fibrosis (IPF), non-specific interstitial pneumonia, respiratory bronchiolitis associated interstitial lung disease, desquamative interstitial pneumonia, cryptogenic orgainizing pneumonia, acute interstitial pneumonia and lymphocytic interstitial pneumonia, lymangioleiomyomatosis, pulmonary alveolar proteinosis, Langerhan's cell histiocytosis, pleural parenchymal fibroelastosis, interstitial lung diseases of known cause, such as interstitial pneumonitis as a result of occupational exposures such as asbestosis, silicosis, miners lung (coal dust), farmers lung (hay and mould), Pidgeon fanciers lung (birds) or other occupational airbourne triggers such as metal dust or mycobacteria, or as a result of treatment such as radiation, methotrexate, amiodarone, nitrofurantoin or chemotherapeutics, or for granulomatous disease, such as granulomatosis with polyangitis, Churg-Strauss syndrome, sarcoidosis, hypersensitivity pneumonitis, or interstitial pneumonitis caused by different origins, e g. aspiration, inhalation of toxic gases, vapors, bronchitis or pneumonitis or interstitial pneumonitis caused by heart failure, X-rays, radiation, chemotherapy, M. boeck or sarcoidosis, granulomatosis, cystic fibrosis or mucoviscidosis, or alpha-1-antitrypsin deficiency.
(2) Other fibrotic diseases such as hepatic bridging fibrosis, liver cirrhosis, non-alcoholic steatohepatitis (NASH), atrial fibrosis, endomyocardial fibrosis, old myocardial infarction, glial scar, arterial stiffness, arthrofibrosis, Dupuytren's contracture, keloid, scleroderma/systemic sclerosis, mediastinal fibrosis, myelotibrosis, Peyronie's disease, nephrogenic systemic fibrosis, retroperitoneal fibrosis, adhesive capsulitis; spontaneous acute exacerbations in pulmonary fibrosis and progressive pulmonary fibrosis or induced by infection, microaspiration, surgical lung biopsy, surgical resection, bronchoscopy (BAL, cryobiopsy), air pollution, prior exacerbation and medications.
(3) Leukemia, acute myeloid leukemia (AML), acute promyelocytic leukemia (APL), chronic myeloid leukemia (CML), acute lymphoblastic leukemia (ALL), T-cell acute lymphoblastic leukemia (T-ALL), lymphoma, B-cell lymphoma, T-cell lymphoma, Hodgkin's disease, non-Hodgkin's lymphoma (NHL), hairy cell lymphoma, Burkett's lymphoma, multiple myeloma (MM), myelodysplastic syndrome, solid cancer, lung cancer, adenocarcinoma, small cell lung cancer (SCLC), non-small cell lung cancer (NSCLC), mediastinum cancer, peritoneal cancer, mesothelioma, gastrointestinal cancer, gastric cancer, stomach cancer, bowel cancer, small bowel cancer, large bowel cancer, colon cancer, colon adenocarcinoma, colon adenoma, rectal cancer, colorectal cancer, leiomyosarcoma, breast cancer, gynaecological cancer, genito-urinary cancer, ovarian cancer, endometrial cancer, cervical cancer, prostate cancer, testicular cancer, seminoma, teratocarcinoma, liver cancer, kidney cancer, bladder cancer, urothelial cancer, biliary tract cancer, pancreatic cancer, exocrine pancreatic carcinoma, esophageal cancer, nasopharyngeal cancer, head and neck squamous cell carcinoma (HNSCC), skin cancer, squamous cancer, squamous cell carcinoma, Kaposi's sarcoma, melanoma, malignant melanoma, xeroderma pigmentosum, keratoacanthoma, bone cancer, bone sarcoma, osteosarcoma, rhabdomyosarcoma, fibrosarcoma, thyroid gland cancer, thyroid follicular cancer, adrenal gland cancer, nervous system cancer, brain cancer, astrocytoma, neuroblastoma, glioma, schwannoma, glioblastoma, or sarcoma, gastrointestinal cancer, gastric cancer, stomach cancer, esophageal cancer, head and neck squamous cell carcinoma (HNSCC), breast cancer, colorectal cancer, bowel cancer, large bowel cancer, colon cancer, colon adenocarcinoma, colon adenoma, rectal cancer, ovarian cancer, pancreatic cancer, exocrine pancreatic carcinoma, leukemia, acute myeloid leukemia (AML), myelodysplastic syndrome, lymphoma, B-cell lymphoma, non-Hodgkin's lymphoma (NHL), urothelial cancer, or peritoneal cancer.
(4) Inflammatory, auto-immune or allergic diseases and conditions such as asthma, pediatric asthma, allergic bronchitis, alveolitis, hyperreactive airways, allergic conjunctivitis, bronchiectasis, adult respiratory distress syndrome, bronchial and pulmonary edema, bronchitis or pneumonitis, non-allergic asthma, chronic obstructive pulmonary disease (COPD), acute bronchitis, chronic bronchitis, pulmonary emphysema; autoimmune diseases, such as rheumatoid arthritis, Graves' disease, Sjogren's syndrome psoriatic arthritis, multiple sclerosis, systemic lupus Erythematosus, inflammatory bowel diseases, such as Crohn's disease and ulcerative colitis, scleroderma; psoriasis (including T-cell mediated psoriasis) and inflammatory dermatoses such as an dermatitis, eczema, atopic dermatitis, allergic contact dermatitis, urticaria; vasculitis (e g, necrotizing, cutaneous, and hypersensitivity vasculitis), or erythemanodosum.
(5) Neurodegenerative disorders such as amyotrophic lateral sclerosis, multiple sclerosis, Parkinson's disease, Alzheimer's disease, Huntington's disease, multiple system atrophy, or prion diseases.

In a further aspect the present invention relates to a compound of general formula (I) or a pharmaceutically acceptable salt thereof or a pharmaceutical composition thereof for use in the treatment and/or prevention of above-mentioned diseases and conditions.

In a further aspect the present invention relates to the use of a compound of general formula (I) or a pharmaceutically acceptable salt thereof or a pharmaceutical composition thereof for the preparation of a medicament for the treatment and/or prevention of above-mentioned diseases and conditions.

In a further aspect of the present invention the present invention relates to methods for the treatment or prevention of above-mentioned diseases and conditions, which method comprises the administration of an effective amount of a compound of general formula (I) or a pharmaceutically acceptable salt thereof or a pharmaceutical composition thereof to a human being.

### Combination Therapy

The compounds of the invention may further be combined with one or more, preferably one additional therapeutic agent. According to one embodiment the additional therapeutic agent is selected from the group of therapeutic agents useful in the treatment of diseases or conditions described hereinbefore, in particular associated with cancer, fibrotic diseases, Alzheimer's diseases, atherosclerosis, infectious diseases, chronic kidney diseases and auto-immune disease.

Additional therapeutic agents that are suitable for such combinations include in particular those, which, for example, potentiate the therapeutic effect of one or more active substances with respect to one of the indications mentioned and/or allow the dosage of one or more active substances to be reduced.

Therefore, a compound of the invention may be combined with one or more additional therapeutic agents selected from the group consisting of chemotherapy, targeted cancer therapy, cancer immunotherapy, irradiation, antifibrotic agents, anti-tussive agents, anti-inflammatory agents, anti-atopic dermatitis, and broncho dilators.

Chemotherapy is a type of cancer therapy that uses one or more chemical anti-cancer drugs, such as cytostatic or cytotoxic substances, cell proliferation inhibitors, anti-angiogenic substances and the like. Examples include folic acid (Leucovorin), 5-Fluorouracil, Irinotecan, Oxaliplatin, cis-platin Azacytidine, gemcitabine, alkylation agents, antimitotic agents, taxanes and further state-of-the-art or standard-of-care compounds.

Targeted therapy is a type of cancer treatment that uses drugs to target specific genes and proteins that help cancer cells survive and grow. Targeted therapy includes agents such as inhibitors of growth factors (e.g. platelet derived growth factor (PDGF), fibroblast growth factor (FGF), vascular endothelial growth factor (VEGF), epidermal growth factor (EGF), insulin-like growth factors (IGF), human epidermal growth factor (HER, e.g. HER2, HER3, HER4) and hepatocyte growth factor), tyrosine-kinases, KRAS, BRAF, BCR-ABL, mTOR, cyclin-dependent kinases, or MDM2.

Cancer immunotherapy is a type of therapy that uses substances to stimulate or suppress the immune system to help the body fight cancer. Cancer immunotherapy includes a therapeutic antibody, such as: anti-Her2 antibody, an anti-EGFR antibody, and an anti-PDGFR antibody; an anti-GD2 (Ganglioside G2) antibody. Examples include Dinutuximab, Olaratumab, Trastuzumab, Pertuzumab, Ertumaxomab, Cetuximab, Necitumumab, Nimotuzumab, Panitumumab, or rituximab. Cancer immunotherapy also includes a therapeutic antibody which is a checkpoint inhibitor, such as an anti PD1, anti PD-L1 antibody or CTLA4 inhibitor. Examples include Atezolizumab, Avelumab, and Durvalumab, Ipilimumab, nivolumab, or pembrolizumab. Cancer immunotherapy also includes agents which target (inhibit) the CD47-SIRPα signaling axis, such as agents which bind to CD47 or SIRPα. Non-limiting examples include antibodies such as anti-CD47 antibodies and anti-SIRPα antibodies, and recombinant Fc-fusion proteins such as CD47-Fc and SIRPα-Fc. Cancer immunotherapy also includes STING-targeting agent, or T cell engagers, such as blinatumomab.

Antifibrotic agents are for example nintedanib, pirfenidone, phosphodiesterase-IV (PDE4) inhibitors such as roflumilast or specific PDE4b inhibitors like BI 1015550, autotaxin inhibitors such as GLPG-1690 or BBT-877; connective tissue growth factor (CTGF) blocking antibodies such as Pamrevlumab; B-cell activating factor receptor (BAFF-R) blocking antibodies such as Lanalumab, alpha-V/beta-6 blocking inhibitors such as BG-00011/STX-100, recombinant pentraxin-2 (PTX-2) such as PRM-151; c-Jun-N-terminal kinase (JNK) inhibitors such as CC-90001; galectin-3 inhibitors such as TD-139; G-protein coupled receptor 84 (GPR84) inhibitors; G-protein coupled receptor 84/G-protein coupled receptor 40 dual inhibitors such as PBI-4050, Rho Associated Coiled-Coil Containing Protein Kinase 2 (ROCK2) inhibitors such as KD-025, heat shock protein 47 (HSP47) small interfering RNA such as BMS-986263/ND-L02-s0201; Wnt pathway inhibitor such as SM-04646; LD4/PDE3/4 inhibitors such as Tipelukast; recombinant immuno-modulatory domains of histidyl tRNA synthetase(HARS) such as ATYR-1923, prostaglandin synthase inhibitors such as ZL-2102/SAR-191801; 15-hydroxy-eicosapentaenoic acid (15-HEPE e.g. DS-102); Lysyl Oxidase Like 2 (LOXL2) inhibitors such as PAT-1251, PXS-5382/PXS-5338; phosphoinositide 3-kinases (PI3K)/mammalian target of rapamycin (mTOR) dual inhibitors such as HEC-68498; calpain inhibitors such as BLD-2660; mitogen-activated protein kinase kinase kinase (MAP3K19) inhibitors such as MG-S-2525; chitinase inhibitors such as OATD-01,mitogen-activated protein kinase-activated protein kinase 2 (MAPKAPK2) inhibitors such as MMI-0100; transforming growth factor beta I (TGF-beta I) small interfering RNA such as TRKZSO/BNC-1021; or lysophosphatidic acid receptor antagonists such as BMS986278.

The dosage for the combination partners mentioned above is usually ⅕ of the lowest dose normally recommended up to 1/1 of the normally recommended dose.

Therefore, in another aspect, this invention relates to the use of a compound according to the invention in combination with one or more additional therapeutic agents described hereinbefore and hereinafter for the treatment of diseases or conditions which may be affected or which are mediated by QPCT/L, in particular diseases or conditions as described hereinbefore and hereinafter.

In a further aspect this invention relates to a method for treating a disease or condition which can be influenced by the inhibition of QPCT/L in a patient that includes the step of administering to the patient in need of such treatment a therapeutically effective amount of a compound of formula (I) or a pharmaceutically acceptable salt thereof in combination with a therapeutically effective amount of one or more additional therapeutic agents.

In a further aspect this invention relates to the use of a compound of formula (I) or a pharmaceutically acceptable salt thereof in combination with one or more additional therapeutic agents for the treatment of diseases or conditions which can be influenced by the inhibition of QPCT/L in a patient in need thereof.

In yet another aspect the present invention relates to a method for the treatment of a disease or condition mediated by QPCT/L activity in a patient that includes the step of administering to the patient, preferably a human, in need of such treatment a therapeutically effective amount of a compound of the present invention in combination with a therapeutically effective amount of one or more additional therapeutic agents described in hereinbefore and hereinafter.

The use of the compound according to the invention in combination with the additional therapeutic agent may take place simultaneously or at staggered times.

The compound according to the invention and the one or more additional therapeutic agents may both be present together in one formulation, for example a tablet or capsule, or separately in two identical or different formulations, for example as a so-called kit-of-parts.

Consequently, in another aspect, this invention relates to a pharmaceutical composition that comprises a compound according to the invention and one or more additional therapeutic agents described hereinbefore and hereinafter, optionally together with one or more inert carriers and/or diluents.

Other features and advantages of the present invention will become apparent from the following more detailed examples which illustrate, by way of example, the principles of the invention.

### Preparation

The compounds according to the present invention and their intermediates may be obtained using methods of synthesis which are known to the one skilled in the art and described in the literature of organic synthesis. Preferably, the compounds are obtained in analogous fashion to the methods of preparation explained more fully hereinafter, in particular as described in the experimental section. In some cases, the order in carrying out the reaction steps may be varied. Variants of the reaction methods that are known to the one skilled in the art but not described in detail here may also be used.

The general processes for preparing the compounds according to the invention will become apparent to the one skilled in the art studying the following schemes. Any functional groups in the starting materials or intermediates may be protected using conventional protecting groups. These protecting groups may be cleaved again at a suitable stage within the reaction sequence using methods familiar to the one skilled in the art.

The compounds according to the invention are prepared by the methods of synthesis described hereinafter in which the substituents of the general formulae have the meanings given herein before. These methods are intended as an illustration of the invention without restricting its subject matter and the scope of the compounds claimed to these examples.

Where the preparation of starting compounds is not described, they are commercially obtainable or may be prepared analogously to known compounds or methods described herein. Substances described in the literature are prepared according to the published methods of synthesis. Abbreviations are as defined in the Examples section.

Examples 1-25 may be prepared as shown in Scheme I below.

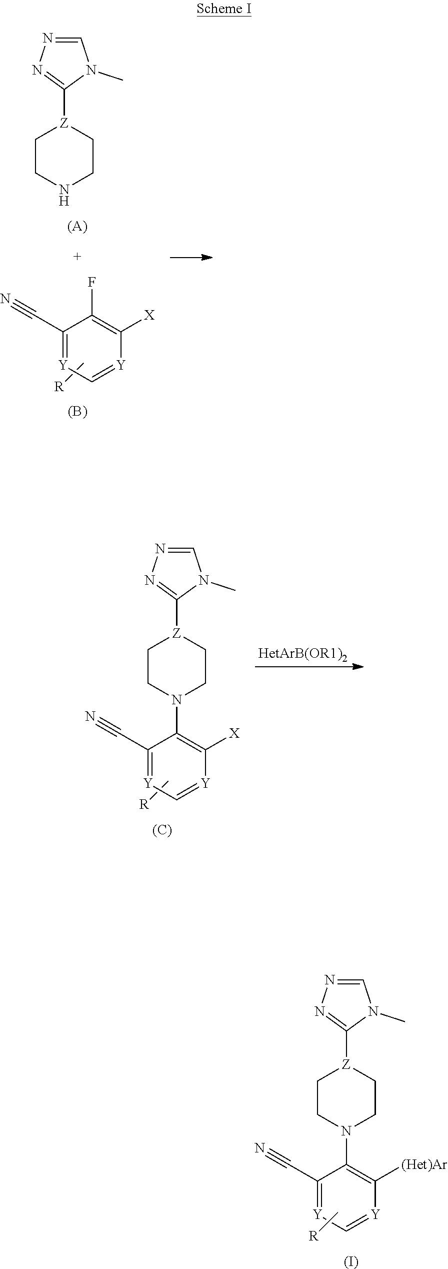

In scheme I, N-Methyl triazolyl piperidine (Z=C—H, C—F, C-Me) (A) undergo a nucleophilic aromatic substitution with heteroaryl fluoride (one Y=N, one Y=CR; X=Cl, Br, I) (B). The reaction can typically be run at ambient temperature or at elevated temperature (up to 110° C.) in the presence of a base (e.g. diisopropylethylamine). The intermediate (C) is then subjected to a Suzuki-cross coupling with a hetero-aryl boronic acid derivative in the presence of a suitable catalyst (e.g. Pd(dppf)Cl₂) and a suitable base at elevated temperature (e.g. 100° C.) to afford compounds of general formula (I).

Intermediates I may be prepared as shown in Scheme II below:

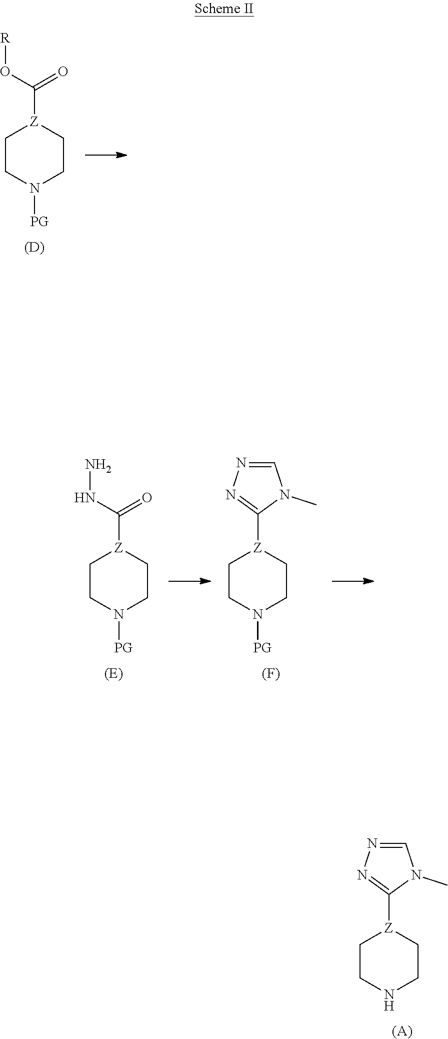

Compounds of formula (A) with Z=C—F, C-Me, C—H can be prepared from the corresponding piperidinyl esters (R=Me, Et) (D) equipped with a suitable protecting group (PG, e.g. BOC) by treatment with a suitable hydrazine source (e.g. N₂H₄*H₂O) at elevated temperature (e.g. 50° C.). The obtained hydrazide (E) is then activated with DMF/DMA at elevated temperature (e.g. 50° C.) and subsequently treated with methyl amine at elevated temperature (e.g. 90° C.) to yield the triazole derivative (F). Compounds of formula (A) can be obtained by cleaving the protecting group under suitable conditions (e.g. 4 M HCl in dioxane for PG=BOC). The obtained hydrochloride salts of (A) are treated with ammonia and passed through a Biotage SNAP Cartridge KP-NH column to release the free amine of (A). Compounds of formula (A) with Z=C—H and C-Me can also be obtained as hydrochlorides salts from commercial sources.

Intermediates II may be prepared as shown in Scheme III below:

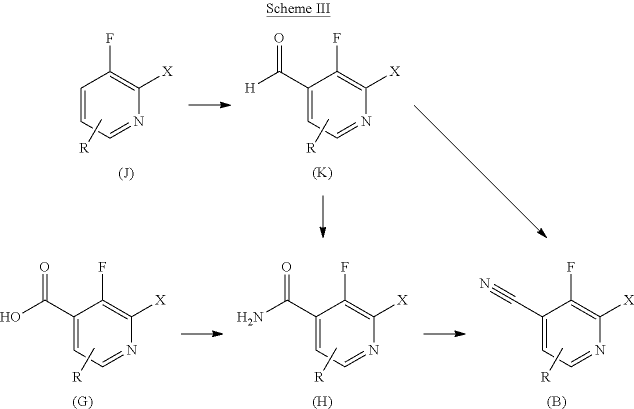

In case of R=H, intermediates of formula (B) can be prepared from the corresponding carboxylic acids (G). The carboxylic acid moiety is transformed into the corresponding amide (H) using a suitable combination of reagents, e.g. 1,1′-carbonyldiimidazole and ammonia at ambient temperature. Compounds of formula (B) are subsequently obtained by treatment of (H) with a suitable dehydrating agent, e.g. Burgess reagent at ambient temperature. In case of R=Me or CF₃, deprotonation of pyridine (J) at low temperature (e.g. −65° C.) and quenching with DMF yields the corresponding aldehydes (K). In case of R=Me, aldehyde (K) can be transformed directly into nitrile (B) by using ammonia in combination with a suitable oxidant (e.g. iodine) at ambient temperature. In case of R=CF₃, aldehyde (K) can be transformed into the amide (H) using a suitable reagent, e.g. phenyltrimethylammonium tribromide, at ambient temperature and further into the nitrile (B) as described above for R=H.

### EXAMPLES

### Preparation

The compounds according to the invention and their intermediates may be obtained using methods of synthesis which are known to the one skilled in the art and described in the literature of organic synthesis for example using methods described in “Comprehensive Organic Transformations”, 2nd Edition, Richard C. Larock, John Wiley & Sons, 2010, and “March's Advanced Organic Chemistry”, 7th Edition, Michael B. Smith, John Wiley & Sons, 2013. Preferably the compounds are obtained analogously to the methods of preparation explained more fully hereinafter, in particular as described in the experimental section. In some cases the sequence adopted in carrying out the reaction schemes may be varied. Variants of these reactions that are known to the skilled artisan but are not described in detail herein may also be used. The general processes for preparing the compounds according to the invention will become apparent to the skilled man on studying the schemes that follow. Starting compounds are commercially available or may be prepared by methods that are described in the literature or herein, or may be prepared in an analogous or similar manner. Before the reaction is carried out, any corresponding functional groups in the starting compounds may be protected using conventional protecting groups. These protecting groups may be cleaved again at a suitable stage within the reaction sequence using methods familiar to the skilled man and described in the literature for example in “Protecting Groups”, 3rd Edition, Philip J. Kocienski, Thieme, 2005, and “Protective Groups in Organic Synthesis”, 4th Edition, Peter G. M. Wuts, Theodora W. Greene, John Wiley & Sons, 2006. The terms “ambient temperature” and “room temperature” are used interchangeably and designate a temperature of about 20° C., e.g. between 19 and 24° C.

Abbreviations:

| | |
|---|---|
| ACN | acetonitrile |
| Aq. | Aqueous |
| BOC | tert-butyloxycarbonyl |
| ° C. | Degree celsius |
| CDI | Carbonyl diimidazole |
| CyH/CH | cyclohexane |
| conc. | Concentrated |
| DCM | dichloromethane |
| DIPEA | N,N-diisopropylethylamine |
| DMA | N,N-dimethylacetamide |
| DMF | N,N-dimethylformamide |
| DMSO | dimethyl sulfoxide |
| Dppf | 1,1′-Bis(diphenylphosphino)ferrocene |
| ESI-MS | Electrospray ionisation mass spectrometry |
| EtOAc | ethyl acetate |
| EtOH | ethanol |
| ex | example |
| eq | equivalent |
| FA | formic acid |
| h | hour |
| HATU | 1-[Bis(dimethylamino)methylene]-1H-1,2,3- |
| | triazolo[4,5-b]pyridinium |
| | 3-oxid hexafluorophosphate |
| HCl | Hydrochloric acid |
| HPLC | High performance liquid chromatography |
| Int. | Intermediate |
| K₂CO₃ | potassium carbonate |
| K(OtBu) | Potassium tert. butoxide |
| L | liter |
| LDA | Lithium diisopropylamide |
| LiOH*H₂O | Lithium hydroxide monohydrate |
| M | Molar (mol/L) |
| MeOH | methanol |
| MgSO₄ | magnesium sulphate |
| min | Minute |
| mL | Milliliter |
| μL | Microliter |
| MTBE | tert-butylmethylether |
| NaOEt | Sodium ethanolate |
| NH₃ | Ammonia |
| PE | Petroleum ether |
| PMB | Para-methoxy benzyl |
| Prep. | Preparative |
| RP | Reversed phase |
| RT | room temperature (about 20° C.) |
| sat. | Saturated |
| TBTU | Benzotriazolyl tetramethyluronium tetrafluoroborate |
| TEA | Triethylamine |
| TFA | trifluoroacetic acid |
| TFAA | trifluoroacetic anhydride |
| THF | Tetrahydrofuran |
| TMS-Cl | Trimethylsilyl chloride |

### PREPARATION OF INTERMEDIATES

### Intermediate 1.1

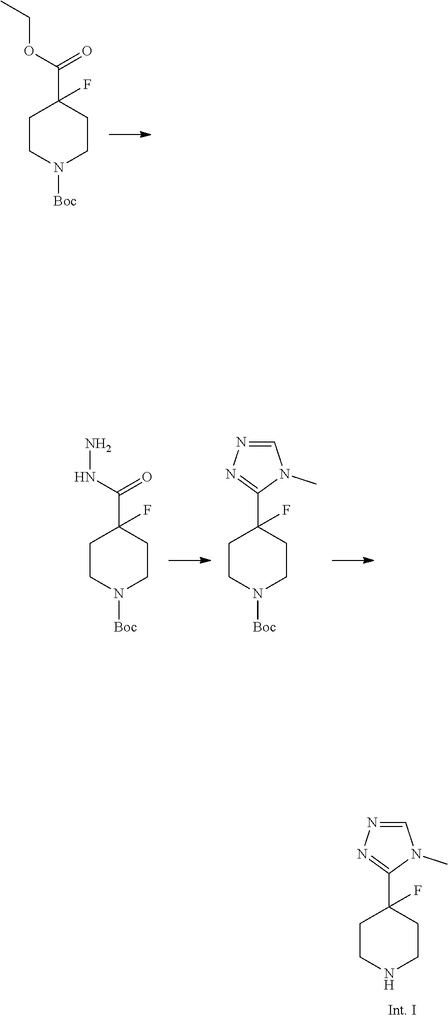

### tert-Butyl 4-fluoro-4-(hydrazinecarbonyl)piperidine-1-carboxylate

1-tert-Butyl 4-ethyl 4-fluoropiperidine-1,4-dicarboxylate (160 g, 0.58 mol) is suspended in ethanol (640 mL) in a round-bottom flask. Hydrazine hydrate (70.6 mL, 1.16 mol) is added to the mixture at ambient temperature. The reaction mixture is heated to 50° C. and stirred for 12 h. After cooling to ambient temperature, the mixture is concentrated under reduced pressure to yield tert-butyl 4-fluoro-4-(hydrazinecarbonyl)piperidine-1-carboxylate in 80% purity.

C₁₁H₂₀FN₃O₃(M=261.3 g/mol)

ESI-MS: 284.2 [M+Na]+

Rt (HPLC): 0.62 min (method A)

### tert-Butyl 4-fluoro-4-(4-methyl-4H-1,2,4-triazol-3-yl)piperidine-1-carboxylate

tert-Butyl 4-fluoro-4-(hydrazinecarbonyl)piperidine-1-carboxylate (135 g, 0.413 mol, 80% purity) is mixed with dioxane (945 mL) in a round-bottom-flask. N,N-Dimethylformamiddimethylacetal (137 mL, 1.03 mol) is added to the mixture at ambient temperature. The reaction mixture is heated to 50° C. and stirred for 1 h. A solution of methylamine (299 g, 30% in EtOH, 2.89 mol) and acetic acid (165 mL, 2.89 mol) are added into the mixture. The resulting reaction mixture is heated to 90° C. and stirred for 11 h. The mixture is concentrated under reduced pressure. The residue is purified by column chromatography (SiO₂, PE/EtOAc gradient 20:1 to 0:1) to obtain tert-butyl 4-fluoro-4-(4-methyl-4H-1,2,4-triazol-3-yl)piperidine-1-carboxylate.

C₁₃H₂₁FN₄O₂(M=284.3 g/mol)

ESI-MS: 285.1 [M+H]⁺

Rt (HPLC): 0.77 min (method A)

### Intermediate I.1: 4-fluoro-4-(4-methyl-4H-1,2,4-triazol-3-yl)piperidine

tert-Butyl 4-fluoro-4-(4-methyl-4H-1,2,4-triazol-3-yl)piperidine-1-carboxylate (90 g, 0.32 mol) is combined with methanol (90 mL) in a round-bottom flask. A solution of HCl (4 M in MeOH, 450 mL, 1.8 mol) is added slowly at ambient temperature. The resulting reaction mixture is stirred at ambient temperature for 12 h. The desired product is collected by filtration, washed with methanol, and dried to yield 4-fluoro-4-(4-methyl-4H-1,2,4-triazol-3-yl)piperidine hydrochloride salt.

The hydrochloride salt (13.5 g) is added to a solution of ammonia in methanol (7 M, 150 mL) and purified by column chromatography (Biotage SNAP Cartridge KP-NH 110 g, gradient DCM/MeOH 4:1 to 7:3)

C₈H₁₃FN₄ (M=184.2 g/mol)

ESI-MS: 185 [M+H]⁺

Rt (HPLC): 0.20 min (method B)

4-(4-Methyl-4H-1,2,4-triazol-3-yl)piperidine (MFCD09055373, CAS: 297172-18-0), 4-(4-methyl-4H-1,2,4-triazol-3-yl)piperidine hydrochloride (MFCD19440843), 1-(4-methyl-4H-1,2,4-triazol-3-yl)piperazine (MFCD27979337, CAS: 67869-95-8), and 4-methyl-4-(4-methyl-4H-1,2,4-triazol-3-yl)piperidine dihydrochloride (MFCD32875324) are obtained from commercial vendors. Hydrochloride salts are converted into the free piperidine or piperazine according to the procedure described for Int.I

### Intermediate II.1

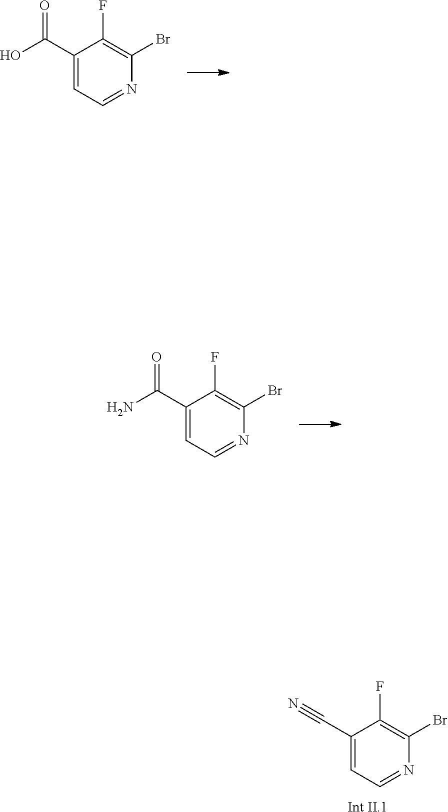

### 2-Bromo-3-fluoropyridine-4-carboxamide

2-Bromo-3-fluoropyridine-4-carboxylic acid (25.0 g, 0.114 mol) is suspended in THF (250 mL), and 1,1′-carbonyldiimazole (22.1 g, 0.136 mol) is added in several smaller portions. The reaction mixture is stirred for 3 h at ambient temperature, before a solution of ammonia (32% in H₂O, 64.8 mL, 0.938 mol) is added slowly. The resulting reaction mixture is stirred for additional 3 h at ambient temperature. The mixture is concentrated, the residue loaded onto Extrelut® and purified by column chromatography (SiO₂, DCM/MeOH 9:1) to yield 2-bromo-3-fluoropyridine-4-carboxamide.

C₆H₄BrFN₂O (M=219.0 g/mol)

ESI-MS: 219/221 [M+H]⁺

Rt (HPLC): 0.28 min (method C)

### Intermediate II.1: 2-bromo-3-fluoropyridine-4-carbonitrile

2-Bromo-3-fluoropyridine-4-carboxamide (17.4 g, 79.5 mmol) is added to dichloromethane (250 mL). Burgess reagent (CAS: 29684-56-8, 22.0 g, 89.5 mmol) is added, and the resulting reaction mixture is stirred for 16 h at ambient temperature. The mixture is concentrated to half of the original volume and purified by column chromatography (SiO₂, DCM) to yield 2-bromo-3-fluoropyridine-4-carbonitrile.

C₆H₂BrFN₂ (M=201.0 g/mol)

ESI-MS: no mass detected

Rt (HPLC): 0.41 min (method F)

1H NMR (400 MHz, DMSO-d6) δ ppm 8.52 (d, J=4.9 Hz, 1H), 8.05 (t, J=4.8 Hz, 1H).

### Intermediate II.2

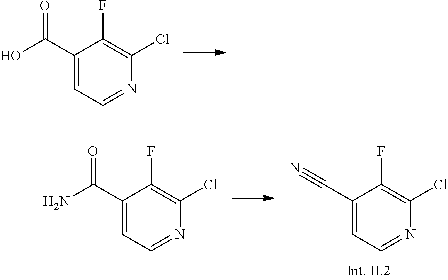

### 2-Chloro-3-fluoropyridine-4-carboxamide

2-Chloro-3-fluoropyridine-4-carboxylic acid (1.00 g, 5.41 mmol) is suspended in THF (25 mL), and 1,1′-carbonyldiimazole (1.05 g, 6.49 mmol) is added in several smaller portions. The reaction mixture is stirred for 2 h at ambient temperature, before a solution of ammonia (32% in H₂O, 3.09 mL, 44.6 mmol) is added slowly. The resulting reaction mixture is stirred for additional 2 h at ambient temperature. The mixture is concentrated, the residue loaded onto Extrelut® and purified by column chromatography (SiO₂, CyH/EtOAc gradient 1:0 to 1:1) to yield 2-chloro-3-fluoropyridine-4-carboxamide.

C₆H₄ClFN₂O (M=174.6 g/mol)

ESI-MS: 174 [M−H]⁻

Rt (HPLC): 0.11 min (method B)

### Intermediate II.2: 2-chloro-3-fluoropyridine-4-carbonitrile

2-Chloro-3-fluoropyridine-4-carboxamide (748 mg, 4.29 mmol) is added to DCM (20 mL). Burgess reagent (CAS: 29684-56-8, 1.26 g, 3.64 mmol) is added, and the resulting reaction mixture is stirred for 16 h at ambient temperature. The mixture is concentrated to half of the original volume and purified by column chromatography (SiO₂, CyH/EtOAc gradient 1:0 to 7:3) to yield 2-chloro-3-fluoropyridine-4-carbonitrile.

C₆H₂ClFN₂ (M=156.5 g/mol)

ESI-MS: no mass detected

Rt (HPLC): 0.41 min (method C)

¹H NMR (400 MHz, DMSO-d₆) δ ppm 8.53 (d, J=4.9 Hz, 1H), 8.06 (t, J=4.7 Hz, 1H)

### Intermediate II.3

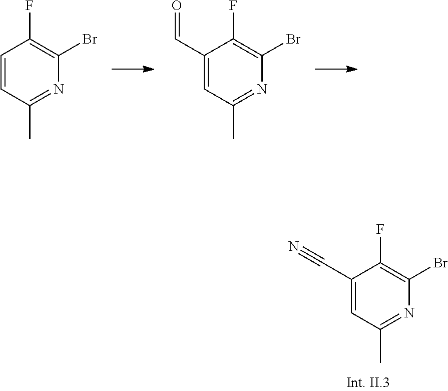

### 2-Bromo-3-fluoro-6-methylpyridine-4-carbaldehyde

Under an argon atmosphere, 2-bromo-3-fluoro-6-methylpyridine (1.00 g, 5.16 mmol) is added to THF (50 mL), and the resulting mixture is cooled to −65° C. A solution of lithium diisopropylamide (1 M in THF, 5.42 mL, 5.42 mmol) is added dropwise and the mixture stirred for another 30 min at −65° C. A solution of DMF (476 μL DMF in 3 mL THF, 6.19 mmol) is added dropwise. The mixture is stirred for 20 min at −65° C. and subsequently allowed to warm to ambient temperature. The reaction is quenched by addition of an aqueous saturated ammonium chloride solution and Me-THF. The phases are separated, the organic phase is washed with water, dried over MgSO₄ and concentrated to yield 2-bromo-3-fluoro-6-methylpyridine-4-carbaldehyde.

C₇H₅BrFNO (M=218.0 g/mol)

ESI-MS: no mass detected

Rt (HPLC): 0.41 min (method C)

¹H NMR (400 MHz, DMSO-d₆) δ ppm 10.16 (s, 1H), 7.64 (d, J=4.6 Hz, 1H), 2.53 (d, J=1.0 Hz, 3H)

### Intermediate II.3: 2-bromo-3-fluoro-6-methylpyridine-4-carbonitrile

2-Bromo-3-fluoro-6-methylpyridine-4-carbaldehyde (460 mg, 2.11 mmol) is added to THF (4.6 mL) and a solution of ammonia (28% in H₂O, 4.6 mL) and iodine (580 mg, 2.28 mmol) are added. The resulting reaction mixture is stirred for 5 h at ambient temperature. The reaction mixture is diluted with ethyl acetate and washed twice with saturated aqueous NH₄Cl solution. The organic phase is concentrated, loaded onto Extrelut® and purified by column chromatography (SiO₂, CyH/EtOAc gradient 1:0 to 4:1) to yield 2-bromo-3-fluoro-6-methylpyridine-4-carbonitrile.

C₇H₄BrFN₂ (M=215.0 g/mol)

ESI-MS: 215/217 [M+H]⁺

Rt (HPLC): 0.40 min (method C)

### Intermediate II.4

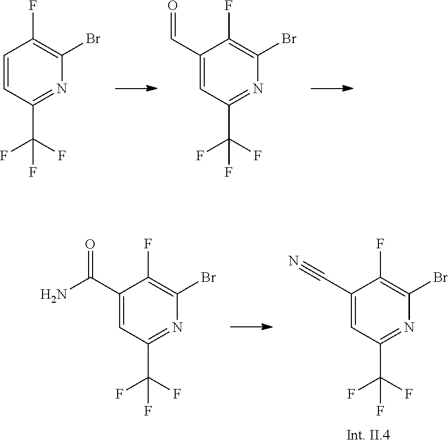 [SMILES: Fc1ccc(C(F)(F)F)nc1Br ; O=Cc1cc(C(F)(F)F)nc(Br)c1F ; NC(=O)c1cc(C(F)(F)F)nc(Br)c1F ; N#Cc1cc(C(F)(F)F)nc(Br)c1F]

### 2-Bromo-3-fluoro-6-(trifluoromethyl)pyridine-4-carbaldehyde

Under an argon atmosphere, 2-bromo-3-fluoro-6-(trifluoromethyl)pyridine (1.00 g, 4.1 mmol) is added to THF (25 mL), and the resulting mixture is cooled to −70° C. A solution of lithium diisopropylamide (1 M in THF, 4.51 mL, 4.51 mmol) is added dropwise, and the mixture is stirred for 45 min at −70° C. DMF (0.378 mL, 4.92 mmol) is added dropwise. The mixture is stirred for additional 30 min at −70° C. The reaction is quenched by addition of acetic acid (800 μL) and diluted with water and ethyl acetate. The organic phase is separated, dried over MgSO₄, and concentrated. The residue is purified by column chromatography (SiO₂, CyH/EtOAc gradient 1:0 to 4:1) to yield 2-bromo-3-fluoro-6-(trifluoromethyl)pyridine-4-carbaldehyde.

C₇H₂BrF₄NO (M=272.0 g/mol)

ESI-MS: no mass detected

Rt (HPLC): 0.48 min (method E)

¹H NMR (400 MHz, DMSO-d₆) δ ppm 10.19 (s, 1H), 8.26 (d, J=4.3 Hz, 1H).

### 2-Bromo-3-fluoro-6-(trifluoromethyl)pyridine-4-carboxamide

Ammonium acetate (5.53 g, 71.8 mmol) and 2-bromo-3-fluoro-6-(trifluoromethyl)pyridine-4-carbaldehyde (2.17 g, 7.18 mmol) are mixed, and acetonitrile (44 mL) is added. Phenyltrimethylammonium tribromide (5.57 g, 14.4 mmol) is added in small portions, and the resulting reaction mixture is stirred for 72 h at ambient temperature. The mixture is filtered, and the residue is washed with acetonitrile, purified by column chromatography (dry load with Celite®, SiO₂, CyH/EtOAc gradient 1:0 to 7:3) to yield the desired product.

C₇H₃BrF₄N₂O (M=287.0 g/mol)

ESI-MS: 285/287 [M−H]⁻

Rt (HPLC): 0.50 min (method B)

### Intermediate II.4: 2-bromo-3-fluoro-6-(trifluoromethyl)pyridine-4-carbonitrile

Product of the previous step, 2-bromo-3-fluoro-6-(trifluoromethyl)pyridine-4-carboxamide (975 mg, 3.40 mmol) is suspended in dichloromethane (80 mL), and Burgess reagent (CAS: 29684-56-8, 1.25 g, 5.10 mmol) is added at ambient temperature. The resulting reaction mixture is stirred for 40 h and then directly purified by column chromatography (dry load with Celite®, SiO₂, CyH/EtOAc gradient 1:0 to 9:1) to yield the desired product.

C₇HBrF₄N₂(M=268.9 g/mol)

ESI-MS: 268/270 [M+H]⁺

Rt (HPLC): 0.59 min (method C)

### General Procedure for the Synthesis of Intermediates III

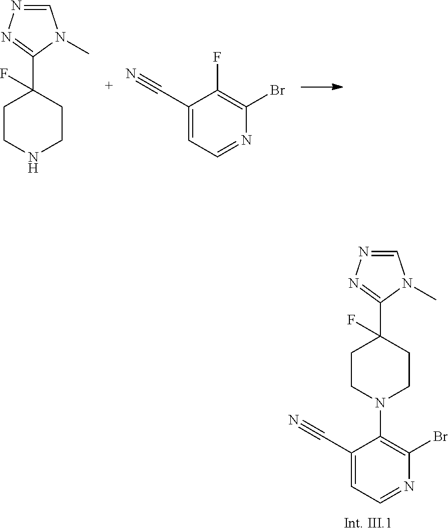

### 2-Bromo-3-[4-fluoro-4-(4-methyl-4H-1,2,4-triazol-3-yl)piperidin-1-yl]pyridine-4-carbonitrile

Int. II.1 (500 mg, 2.49 mmol) and Int. I.1 (687 mg, 3.73 mmol) are suspended in DMSO (5.0 mL), and DIPEA (861 μL, 4.98 mmol) is added at ambient temperature. The resulting mixture stirred for 72 h at ambient temperature. The mixture is diluted with water (0.1 mL), and the precipitate is collected by filtration, washed with water, and dried to yield the desired product 2-bromo-3-[4-fluoro-4-(4-methyl-4H-1,2,4-triazol-3-yl)piperidin-1-yl]pyridine-4-carbonitrile, which is used in the next step without further purification.

C₁₄H₁₄BrFN₆ (M=365.2 g/mol)

ESI-MS: 365/367 [M+H]⁺

Rt (HPLC): 0.42 min (method C)

| | | | | Molecular Formula |
|---|---|---|---|---|
| | | | | (MW) |
| | | | | ESI-MS |
| | | | Deviation from | HPLC retention |
| Int. | Starting material | Structure | general procedure | time (method) |
| III.2 | 4-(4-methyl-4H- 1,2,4-triazol-3- yl)piperidine + II.1 | 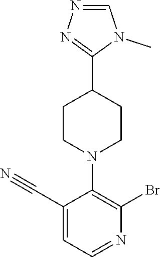 [SMILES: Cn1cnnc1C1CCN(c2c(C#N)ccnc2Br)CC1] | 1 h @ 100° C.; purifi- cation by preparative HPLC (Xbridge C18, acetonitrile/water gra- dient containing 0.1% NH₃) | C₁₄H₁₅BrN₆ (M = 347.2 g/mol) ESI-MS: 347/349 [M + H]⁺ Rt (HPLC): 0.34 min (method C) |
| III.3 | 4-methyl-4-(4- methyl-1,2,4- triazol-3- yl)piperidine + II.1 | 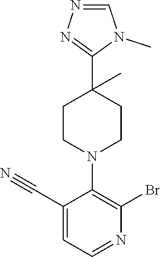 [SMILES: Cn1cnnc1C1(C)CCN(c2c(C#N)ccnc2Br)CC1] | 1 h @ 100° C.; purifi- cation by preparative HPLC (Xbridge C18, acetonitrile/water gra- dient containing 0.1% TFA) | C₁₅H₁₇BrN₆ (M = 361.2 g/mol) ESI-MS: 361/363 [M + H]⁺ Rt (HPLC): 0.41 min (method C) |
| III.4 | Int. I.1 + II.2 | 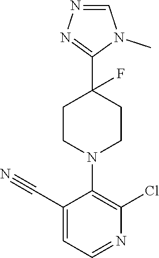 [SMILES: Cn1cnnc1C1(F)CCN(c2c(C#N)ccnc2Cl)CC1] | 16 h @ 10° C.; | C₁₄H₁₄ClFN₆ (M = 320.8 g/mol) ESI-MS: 321 [M + H]⁺ Rt (HPLC): 0.43 min (method C) |
| III.5 | Int. I.1 + Int. II.3 | 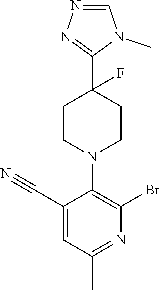 [SMILES: Cc1cc(C#N)c(N2CCC(F)(c3nncn3C)CC2)c(Br)n1] | 1.2 equiv of Int. I.1; 5 h @ 50° C.; Purification by prepar- ative HPLC (Xbridge C18, acetonitrile/water gradient containing 0.1% TFA) | C₁₅H₁₆BrFN₆ (M = 379.2 g/mol) ESI-MS: 379/381 [M + H]⁺ Rt (HPLC): 0.83 min (method F) |
| III.6 | Int. I.1 + Int. II.4 | 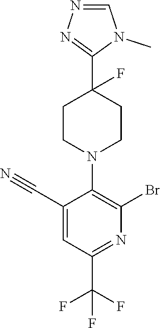 [SMILES: Cn1cnnc1C1(F)CCN(c2c(C#N)cc(C(F)(F)F)nc2Br)CC1] | 1.2 equiv of Int. I.1; 1.5 h @ 10° C.; Purification by prepar- ative HPLC (Xbridge C18, acetonitrile/water gradient containing 0.1% NH₃) | C₁₅H₁₃BrF₄N₆ (M = 433.2 g/mol) ESI-MS: 433/435 [M + H]⁺ Rt (HPLC): 0.54 min (method C) |

### Intermediate IV.1

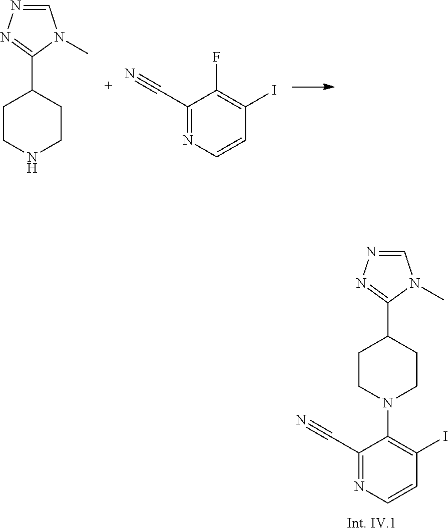

4-(4-Methyl-4H-1,2,4-triazol-3-yl)piperidine hydrochloride (180 mg, 0.89 mmol), 3-fluoro-4-iodoopicolinonitrile (200 mg, 0.81 mmol) and potassium carbonate (334 mg, 2.42 mmol) are suspended in DMF (4.0 mL), and the resulting reaction mixture is heated to 90° C. and stirred for 18 h. After cooling to ambient temperature, the mixture is concentrated, redissolved in a mixture of acetonitrile, water, and methanol, filtered, and purified by preparative HPLC (XBridge C18, acetonitrile/water gradient containing 0.1% NH₃) to yield the desired product along with a by-product resulting from iodine replacement.

C₁₄H₁₅IN₆ (M=394.2 g/mol)

ESI-MS: 395 [M+H]⁺

Rt (HPLC): 0.38 min (method C)

| | | | | Molecular Formula |
|---|---|---|---|---|
| | | | | (MW) |
| | | | Deviation | ESI-MS |
| | | | from general | HPLC retention time |
| Int. | Starting materials | Structure | procedure | (method) |
| IV.2 | Int. I + 3-fluoro-4- iodopyridine-2- carbonitrile | 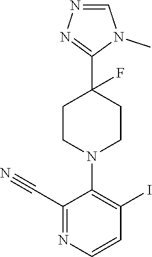 [SMILES: Cn1cnnc1C1(F)CCN(c2c(I)ccnc2C#N)CC1] | 4 eq. of potas- sium carbonate used | C₁₄H₁₄IFN₆ (M = 412.2 g/mol) ESI-MS: 413 [M + H]⁺ Rt (HPLC): 0.43 min (method C) |
| IV.3 | 4-methyl-4-(4- methyl-1,2,4- triazol-3- yl)piperidine dihydrochloride + 3-fluoro-4-io- dopyridine-2-car- bonitrile | 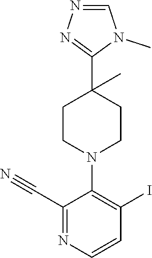 [SMILES: Cn1cnnc1C1(C)CCN(c2c(I)ccnc2C#N)CC1] | 4 eq. of potas- sium carbonate used | C₁₅H₁₇IN₆ (M = 408.2 g/mol) ESI-MS: 409 [M + H]⁺ Rt (HPLC): 0.40 min (method C) |

### Intermediate V.1

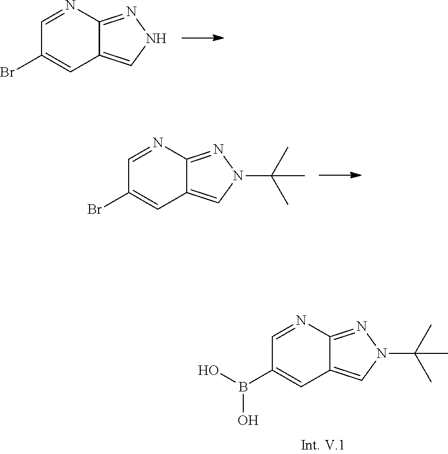 [SMILES: Brc1cnc2n[nH]cc2c1 ; CC(C)(C)n1cc2cc(Br)cnc2n1 ; CC(C)(C)n1cc2cc(B(O)O)cnc2n1]

### 5-Bromo-2-tert-butyl-2H-pyrazolo[3,4-b]pyridine

5-Bromo-1H-pyrazolo[3,4-b]pyridine (4.0 g, 19.8 mmol) is suspended in toluene (23 mL), and tert-butyl acetate (26.6 mL, 198 mmol) is added. Methanesulfonic acid (1.3 mL, 19.8 mmol) is added slowly. The resulting reaction mixture is heated to 80° C. and stirred for 1 h. After cooling to ambient temperature, additional methanesulfonic acid (1.3 mL, 19.8 mmol) added, and the reaction mixture is heated to 80° C. and stirred for 1 h. After cooling to ambient temperature, the reaction mixture is concentrated and purified by preparative HPLC (XBridge C18, ACN/water gradient containing 0.1% TFA) to yield the 5-bromo-2-tert-butyl-2H-pyrazolo [3,4-b]pyridine.

C₁₀H₁₂BrN₃ (M=254.1 g/mol)

ESI-MS: 254/256 [M+H]⁺

Rt (HPLC): 0.50 min (method C)

### Intermediate V.1

5-Bromo-2-tert-butyl-2H-pyrazolo[3,4-b]pyridine (1.50 g, 3.87 mmol), bis(pinacoloto)diboron (1.21 g, 4.78 mmol), and potassium acetate (763 mg, 7.77 mmol) are added to 1,4-dioxane (15 mL), and the resulting mixture is degassed by passing an argon stream through the mixture for 10 min. [1,1′-Bis-(diphenylphosphino)-ferrocen]-dichloro-palladium(II) dichloromethane complex (Pd(dppf)Cl₂*CH₂Cl₂, CAS: 95464-05-4) (190 mg, 0.232 mmol) is added, and the mixture is degassed for additional 3 min. The mixture is then heated to 110° C. and stirred at this temperature for 4 h. After cooling to ambient temperature, the mixture is concentrated, and the residue is dissolved in a ACN/water mixture, filtered, and purified by preparative HPLC (XBridge C18, ACN/water gradient containing 0.1% TFA) to yield {2-tert-butyl-2H-pyrazolo[3,4-b]pyridin-5-yl}boronic acid.

C₁₀H₁₄BN₃O₂(M=219.1 g/mol)

ESI-MS: 220 [M+H]⁺

Rt (HPLC): 0.27 min (method C)

### Intermediate V.2

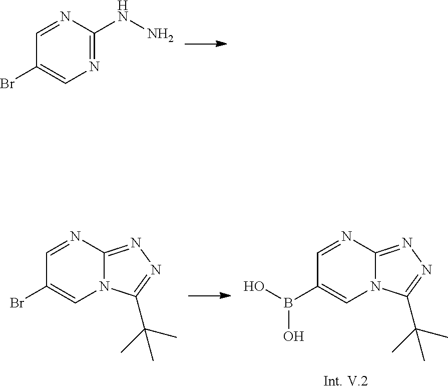 [SMILES: NNc1ncc(Br)cn1 ; CC(C)(C)c1nnc2ncc(Br)cn12 ; CC(C)(C)c1nnc2ncc(B(O)O)cn12]

### 6-Bromo-3-tert-butyl-[1,2,4]triazolo[4,3-a]pyrimidine

5-Bromo-2-hydrazinopyrimidine (200 mg, 1.01 mmol) and pivaloyl chloride (985 mg, 8.09 mmol) are added into a microwave vial, which is subsequently sealed. The mixture is heated to 120° C. and stirred at this temperature for 22 h. Water and ACN are added, and the mixture is concentrated. The residue is taken up with ACN and purified by preparative HPLC (XBridge C18, ACN/water gradient containing 0.1% TFA) to yield the desired compound.

C₉H₁₁BrN₄ (M=255.1 g/mol)

ESI-MS: 255/257 [M+H]⁺

Rt (HPLC): 0.46 min (method C)

### Intermediate V.2

6-Bromo-3-tert-butyl-[1,2,4]triazolo[4,3-a]pyrimidine (75.0 mg, 0.294 mmol), bis-(pinacolato)-diboron (100 mg, 0.394 mmol), and potassium acetate (90.0 mg, 0.917 mmol) are added to 1,4-dioxane (1.5 mL). The mixture is degassed by passing an argon stream through the mixture for 15 min. Bis(triphenylphosphine)palladium(II) dichloride (CAS: 13965-03-2; 20.4 mg, 0.029 mmol) is added, and the mixture is further degassed for 3 min. The resulting reaction mixture is heated to 60° C. and stirred at this temperature for 20 h. After cooling to ambient temperature, the mixture is diluted with EtOAc and filtered. The filtrate is concentrated and taken up with a mixture of ACN, water, and TFA. It is then purified by preparative HPLC (Sunfire C18, ACN/water gradient containing 0.1% TFA) to yield the desired compound.

C₉H₁₃BN₄O₂(M=220.0 g/mol)

ESI-MS: 221 [M+H]⁺

Rt (HPLC): 0.34 min (method C)

### Intermediate V.3

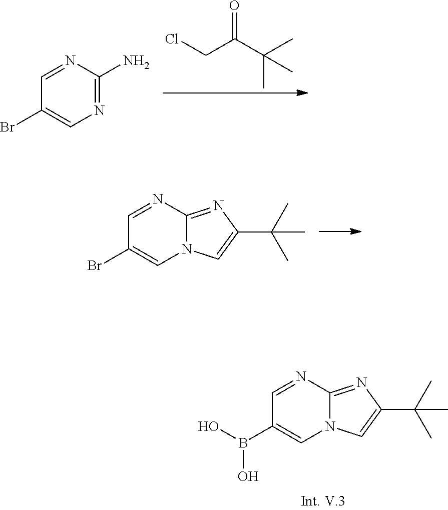

### 6-Bromo-2-tert-butylimidazo[1,2-a]pyrimidine

2-Amino-5-bromopyrimidine (250 mg, 1.41 mmol) and 1-chloropinacolone (285 μL, 2.11 mmol) are added to ethanol (2.0 mL), and the resulting reaction mixture is stirred for 96 h at 90° C. After cooling to ambient temperature, the mixture is diluted with ACN and purified by column chromatography (SiO₂, DCM/MeOH gradient 1:0 to 9:1) to yield the desired product.

C₁₀H₁₂BrN₃ (M=254.1 g/mol)

ESI-MS: 254/256 [M+H]⁺

Rt (HPLC): 0.28 min (method C)

### Intermediate V.3

6-Bromo-2-tert-butylimidazo[1,2-a]pyrimidine (144 mg, 0.567 mmol), bis-(pinacolato)-diboron (215 mg, 0.850 mmol), and potassium acetate (167 mg, 1.70 mmol) are added to 1,4-dioxane (1.0 mL). The mixture is degassed by passing an argon stream through the mixture for 10 min. Bis(triphenylphosphine)palladium(II) dichloride (CAS: 13965-03-2; 39.8 mg, 56.7 μmol) is added, and the mixture is further degassed for 3 min. The resulting reaction mixture is heated to 90° C. and stirred at this temperature for 5 h. After cooling to ambient temperature, the mixture is concentrated. The residue is taken up with a mixture of water and ACN and then purified by preparative HPLC (XBridge C18, ACN/water gradient containing 0.1% TFA) to yield the desired compound.

C₁₀H₁₄BN₃O₂(M=219.1 g/mol)

ESI-MS: 220 [M+H]⁺

Rt (HPLC): 0.25 min (method C)

### Intermediate V.4

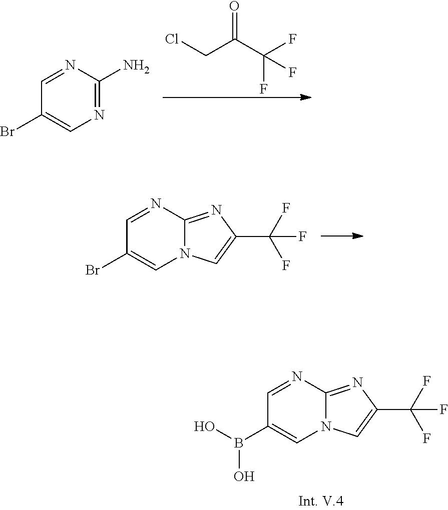 [SMILES: Nc1ncc(Br)cn1 ; O=C(CCl)C(F)(F)F ; FC(F)(F)c1cn2cc(Br)cnc2n1 ; OB(O)c1cnc2nc(C(F)(F)F)cn2c1]

### 6-Bromo-2-(trifluoromethyl)imidazo[1,2-a]pyrimidine

2-Amino-5-bromopyrimidine (1.0 g, 5.6 mmol) and 1-chloro-3,3,3-trifluoroacetone (889 μL, 8.45 mmol) are added to ethanol (8 mL), and the resulting reaction mixture is stirred for 120 h at 90° C. After cooling to ambient temperature, the mixture is concentrated and loaded onto Extrelut®. It is then purified by column chromatography (SiO₂, DCM/MeOH gradient 1:0 to 9:1) to yield the desired product.

C₇H₃BrF₃N₃(M=266.0 g/mol)

ESI-MS: 266/268 [M+H]⁺

Rt (HPLC): 0.38 min (method C)

### Intermediate V.4

6-Bromo-2-(trifluoromethyl)imidazo[1,2-a]pyrimidine (373 mg, 1.40 mmol), bis-(pinacolato)-diboron (215 mg, 1.54 mmol), and potassium acetate (412 mg, 4.21 mmol) are added to 1,4-dioxane (1.0 mL). The mixture is degassed by passing an argon stream through the mixture for 10 min. Bis(triphenylphosphine)palladium(II) dichloride (98.4 mg, 0.140 mmol) is added, and the mixture is further degassed for 3 min. The resulting reaction mixture is heated to 90° C. and stirred at this temperature for 5 h. After cooling to ambient temperature, the mixture is concentrated. The residue is taken up with a mixture of water and ACN and purified by preparative HPLC (XBridge C18, ACN/water gradient containing 0.1% TFA) to yield the desired compound.

C₇H₅BF₃N₃O₂ (M=230.9 g/mol)

ESI-MS: 232 [M+H]⁺

Rt (HPLC): 0.29 min (method C)

### Intermediate VI.1

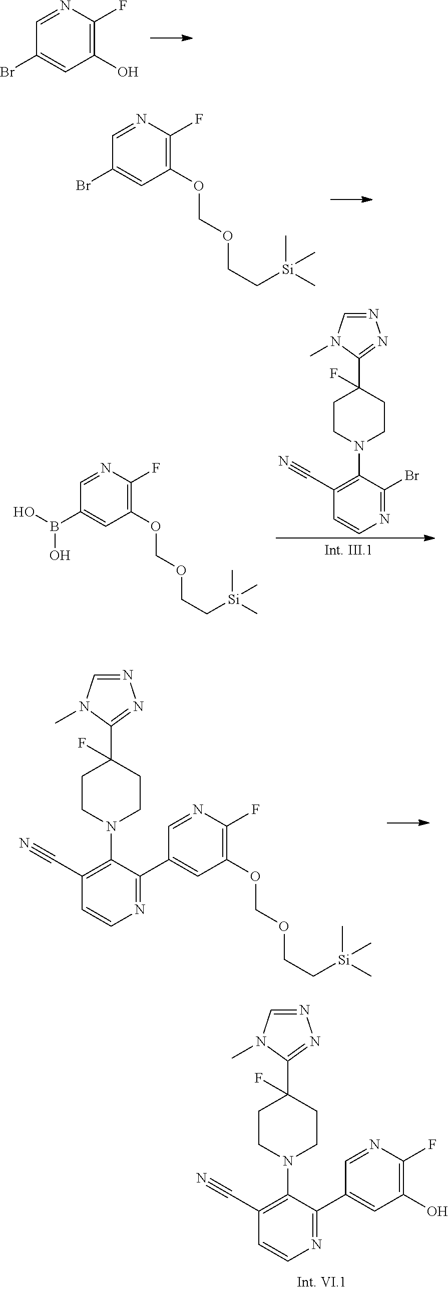

### 5-Bromo-2-fluoro-3-{[2-(trimethylsilyl)ethoxy]methoxy}pyridine

5-Bromo-2-fluoropyridin-3-ol (3.00 g, 15.3 mmol) is added to DCM (40 mL), and DIPEA (5.3 mL, 31 mmol) is added. 2-(Trimethylsilyl)ethoxymethyl chloride (3.15 mL, 16.8 mmol) is added dropwise, and the resulting reaction mixture is stirred for 90 min at ambient temperature. The mixture is concentrated, loaded onto Extrelut®, and purified by column chromatography (SiO₂, CyH/EtOAc 1:0 to 3:1) to yield the desired product.

C₁₁H₁₇BrFNO₂Si (M=322.2 g/mol)

ESI-MS: 322/324 [M+H]⁺

Rt (HPLC): 0.84 min (method C)

### (6-Fluoro-5-{[2-(trimethylsilyl)ethoxy]methoxy}pyridin-3-yl)boronic acid

Under an argon atmosphere, 1,4-dioxane (30 mL) is added to a mixture of 5-bromo-2-fluoro-3-{[2-(trimethylsilyl)ethoxy]methoxy}pyridine (4.80 g, 14.9 mmol), bis(pinacolato)diboron (3.50 g, 13.8 mmol), and potassium acetate (4.39 g, 44.7 mmol). The suspension is degassed by passing an Argon stream through the mixture for 10 min. [1,1′-Bis-(diphenylphosphino)-ferrocen]-dichloro-palladium(II) dichloromethane complex (Pd(dppf)Cl₂*CH₂Cl₂, CAS: 95464-05-4) (545 mg, 0.745 mmol) is added, and the mixture is further degassed for 3 min. The mixture is then heated to 80° C. and stirred at this temperature for 18 h. After cooling to ambient temperature, the mixture is diluted with EtOAc and water. The organic phase is separated, dried over MgSO₄, and concentrated. The residue is purified by preparative HPLC (XBridge C18, ACN/water gradient) to yield the desired product in 80% purity.

C₁₁H₁₉BFNO₄Si (M=287.2 g/mol)

ESI-MS: 288 [M+H]⁺

Rt (HPLC): 0.60 min (method C)

### 6′-Fluoro-3-[4-fluoro-4-(4-methyl-4H-1,2,4-triazol-3-yl)piperidin-1-yl]-5′-{[2-(trimethylsilyl)ethoxy]methoxy}-[2,3′-bipyridine]-4-carbonitrile

Under an argon atmosphere, 1,4-dioxane (3.0 mL) is added to a mixture of intermediate III.1 (211 mg, 0.578 mmol), (6-fluoro-5-{[2-(trimethylsilyl)ethoxy]methoxy}pyridin-3-yl)boronic acid (80% purity, 519 mg, 1.44 mmol), and tetrakis(triphenylphosphine)palladium(0) (66.8 mg, 0.058 mmol). The suspension is degassed by passing an Argon stream through the mixture for 10 min. A solution of cesium carbonate (2 M in H₂O, 867 μL, 1.73 mmol) is added, and the mixture is again degassed for 3 min. The reaction mixture is then heated to 80° C. and stirred at this temperature for 8 h. After cooling to ambient temperature, the mixture is concentrated. The residue is taken up in ACN and purified by preparative HPLC (XBridge C18, ACN/water gradient containing 0.1% TFA) to yield the desired product.

C₂₅H₃₁F₂N₇O₂Si (M=527.6 g/mol)

ESI-MS: 528 [M+H]⁺

Rt (HPLC): 0.67 min (method C)

### Intermediate VI.1

6′-Fluoro-3-[4-fluoro-4-(4-methyl-4H-1,2,4-triazol-3-yl)piperidin-1-yl]-5′-{[2-(trimethylsilyl)ethoxy]methoxy}-[2,3′-bipyridine]-4-carbonitrile (290 mg, 0.45 mmol) is added to dichloromethane and a solution of hydrochlorid acid (4 M in dioxane, 563 μL, 2.25 mmol) is added. The resulting reaction mixture is stirred for 2 h at ambient temperature. The mixture is diluted with MTBE, and the precipitated solid is collected by filtration, washed with MTBE, and dried to yield the desired product.

C₁₉H₁₇F₂N₇O (M=397.4 g/mol)

ESI-MS: 398 [M+H]⁺

Rt (HPLC): 0.39 min (method C)

### Intermediate VI.2

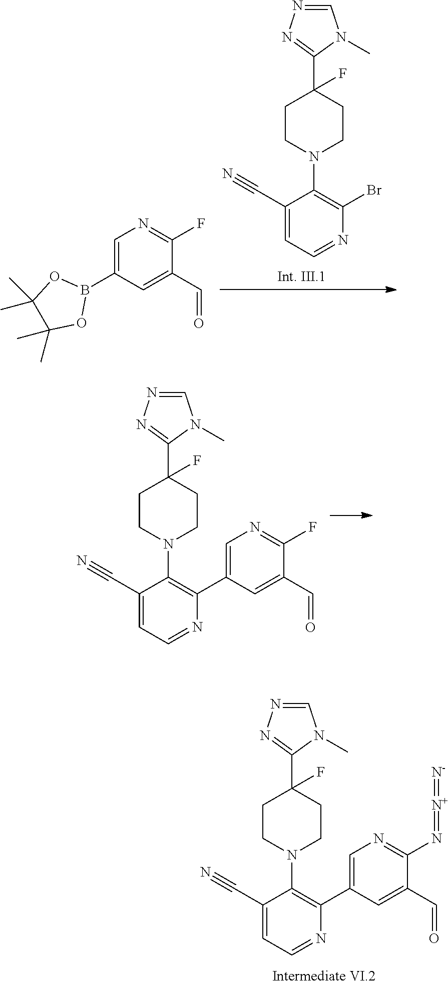 [SMILES: CC1(C)OB(c2cnc(F)c(C=O)c2)OC1(C)C ; Cn1cnnc1C1(F)CCN(c2c(C#N)ccnc2Br)CC1 ; Cn1cnnc1C1(F)CCN(c2c(C#N)ccnc2-c2cnc(F)c(C=O)c2)CC1 ; Cn1cnnc1C1(F)CCN(c2c(C#N)ccnc2-c2cnc(N=[N+]=[N-])c(C=O)c2)CC1]

### 6′-Fluoro-3-[4-fluoro-4-(4-methyl-4H-1,2,4-triazol-3-yl)piperidin-1-yl]-5′-formyl-[2,3′-bipyridine]-4-carbonitrile

Under an argon atmosphere, intermediate III.1 (1.0 g, 2.74 mmol), 2-fluoro-5-(4,4,5,5-tetra-methyl-1,3,2-dioxaborolan-2-yl)nicotinaldehyde (CAS: 1333319-63-3; 796 mg, 3.01 mmol) and a solution of potassium carbonate (2 M in water, 2.74 mL, 5.48 mmol) are added to 1,4-dioxane (30 mL). The mixture is degassed by passing an argon stream through the mixture for 10 min. Then, [1,1′-Bis-(diphenylphosphino)-ferrocen]-dichloro-palladium(II) (Pd(dppf)Cl₂, CAS: 72287-26-4) (200 mg, 0.274 mmol) is added, and the mixture is further degassed for 3 min. The reaction mixture is then heated to 100° C. and stirred at this temperature for 1 h. After cooling to ambient temperature, the mixture is diluted with EtOAc and water. The organic phase is separated, dried over sodium sulfate, and concentrated. The residue is taken up with DMF and purified by preparative HPLC (ZORBAX StableBond C18, ACN/water gradient containing 0.1% TFA) to yield the desired product.

C₂₀H₁₇F₂N₇O (M=409.4 g/mol)

ESI-MS: 410 [M+H]⁺

Rt (HPLC): 0.73 min (method G)

### Intermediate VI.2

6′-Fluoro-3-[4-fluoro-4-(4-methyl-4H-1,2,4-triazol-3-yl)piperidin-1-yl]-5′-formyl-[2,3′-bipyridine]-4-carbonitrile (660 mg, 1.61 mmol) and tetrabutylammonium iodide (59.5 mg, 0.161 mmol) are added to DMSO (20 mL). Sodium azide (212 mg, 3.22 mmol) is then added in small portions at ambient temperature. The resulting reaction mixture is stirred for 1 h at ambient temperature before it is diluted with an aqueous half saturated sodium chloride solution. The organic phase is extracted with EtOAc (3×), and the combined organic layers are dried over sodium sulfate and concentrated to yield the desired product that was used in the next step without further purification.

C₂₀H₁₇FN₁₀O (M=432.4 g/mol)

ESI-MS: 433 [M+H]⁺

Rt (HPLC): 0.66 min (method G)

### Preparation of Final Compounds

### Example 1

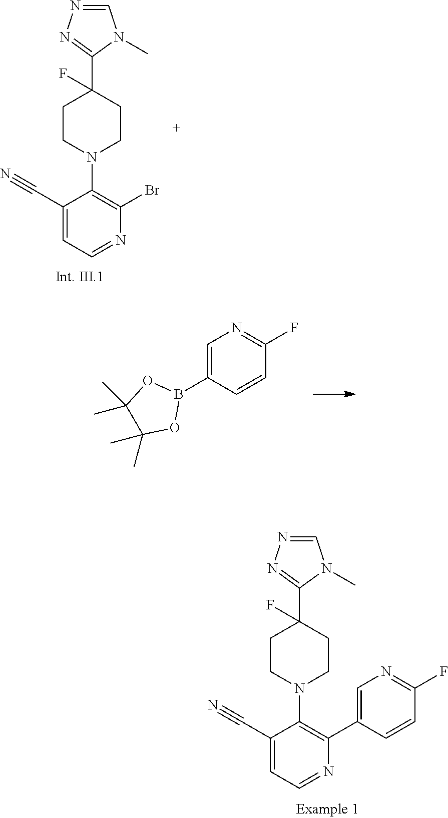 [SMILES: Cn1cnnc1C1(F)CCN(c2c(C#N)ccnc2Br)CC1 ; CC1(C)OB(c2ccc(F)nc2)OC1(C)C ; Cn1cnnc1C1(F)CCN(c2c(C#N)ccnc2-c2ccc(F)nc2)CC1]

Under an argon atmosphere, intermediate III.1 (1.43 g, 3.92 mmol) and 2-fluoropyridine-5-boronic acid pinacol ester (873 mg, 3.92 mmol) are suspended in 1,4-dioxane (15 mL), and a solution of potassium carbonate (2 M in water, 3.9 mL, 7.8 mmol) is added. The resulting mixture is degassed by passing an argon stream through the solution for 15 min. [1,1′-Bis-(diphenylphosphino)-ferrocen]-dichloro-palladium(II) (Pd(dppf)Cl₂, CAS: 72287-26-4) (286 mg, 0.392 mmol) is added, and the mixture is further degassed for 3 min. The reaction mixture is heated to 100° C. and stirred for 5 h. After cooling to ambient temperature, it is diluted with ethyl acetate and filtered over Celite®. The filtrate is concentrated and purified via preparative HPLC (XBridge C18 column, ACN/water gradient containing 0.1% NH₃) to yield the desired compound.

C₁₉H₁₇F₂N₇ (M=381.4 g/mol)

ESI-MS: 382 [M+H]⁺

Rt (HPLC): 0.40 min (method C)

¹H NMR (400 MHz, DMSO-d₆) δ ppm: 8.54 (d, J=4.9 Hz, 1H), 8.50 (d, J=2.3 Hz, 1H), 8.48 (s, 1H), 8.25 (td, J=8.2, 2.5 Hz, 1H), 7.82 (d, J=4.8 Hz, 1H), 7.34 (dd, J=8.4, 2.6 Hz, 1H), 3.73 (d, J=1.6 Hz, 3H), 3.15-3.28 (m, 4H), 2.09-2.27 (m, 4H)

### Examples Synthesized Analogous to the Procedure Described for Example 1

| | Starting | | Deviation from general |
|---|---|---|---|
| example | materials | Structure | procedure |
| 2 | Int. III.2 + 2-fluoro- pyridine-5- boronic acid pinacol ester | 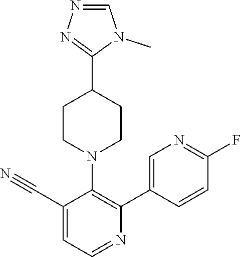 [SMILES: Cn1cnnc1C1CCN(c2c(C#N)ccnc2-c2ccc(F)nc2)CC1] | 1 h at 100° C.; purification by preparative HPLC (XBridge C18, MeOH/water gradient containing 0.1% NH₃) |
| 3 | Int. III.3 + 2-fluoro- pyridine-5- boronic acid pinacol ester | 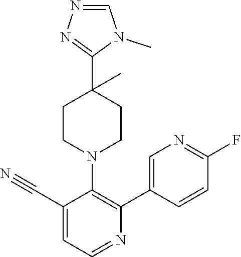 [SMILES: Cn1cnnc1C1(C)CCN(c2c(C#N)ccnc2-c2ccc(F)nc2)CC1] | purification by preparative HPLC (XBridgeC18, ACN/water gradient contain- ing 0.1% TFA; then XBridge C18, ACN/water gradient containing 0.1% NH₃) |
| 4 | Int. III.2 + 2-fluoro3-iso- propoxypyri- dine-5-boronic acid pincacol ester | 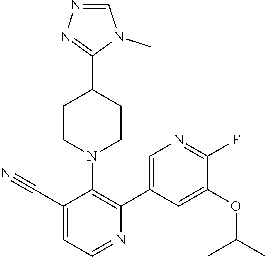 [SMILES: CC(C)Oc1cc(-c2nccc(C#N)c2N2CCC(c3nncn3C)CC2)cnc1F] | 1 h at 100° C.; purified by preparative HPLC (XBridgeC18, MeOH/water gradient containing 0.1% NH₃; then XBridgeC18, ACN/water gradient contain- ing 0.1% NH₃) |
| 5 | Int. III.2 + 2-fluoro3- methoxy- pyridine- 5-boronic acid | 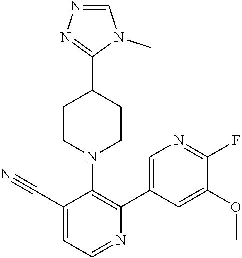 [SMILES: COc1cc(-c2nccc(C#N)c2N2CCC(c3nncn3C)CC2)cnc1F] | 1 h at 100° C.; purified by preparative HPLC (XBridgeC18, ACN/water gradient containing 0.1% NH₃; then XBridge C18, ACN/water gradient contain- ing 0.1% TFA) |
| 6 | Int. III.1 + 2-fluoro3- methylpyridine- 5-boronic acid | 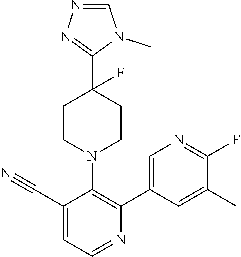 [SMILES: Cc1cc(-c2nccc(C#N)c2N2CCC(F)(c3nncn3C)CC2)cnc1F] | 1.5 eq. of boronic acid used |
| 7 | Int. III.1 + 2-fluoro3- methoxy- pyridine-5- boronic acid | 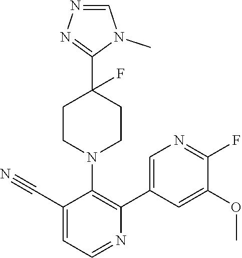 [SMILES: COc1cc(-c2nccc(C#N)c2N2CCC(F)(c3nncn3C)CC2)cnc1F] | Na₂CO₃ was used instead of K₂CO₃; 1h at 100° C.; puri- fied by preparative HPLC (Stablebond C18, ACN/water gradient containing 0.1% TFA) |
| 8 | Int. III.1 + Int. V.1 | 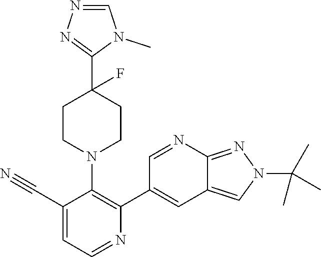 [SMILES: Cn1cnnc1C1(F)CCN(c2c(C#N)ccnc2-c2cnc3nn(C(C)(C)C)cc3c2)CC1] | 1.5 eq of Int. V.1 |
| 9 | Int. IV.1 + 2-fluoro- pyridine-5- boronic acid | 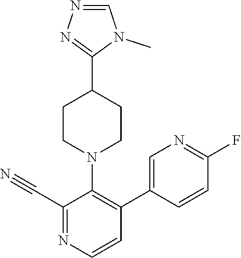 [SMILES: Cn1cnnc1C1CCN(c2c(-c3ccc(F)nc3)ccnc2C#N)CC1] | 1.2 eq 2-fluoropyridine-5- boronic acid; 4.0 eq. K₂CO₃ solution; 30 min at 100° C. |
| 10 | Int. IV.2 + 2-fluoro- pyridine-5- boronic acid | 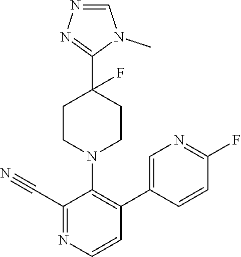 [SMILES: Cn1cnnc1C1(F)CCN(c2c(-c3ccc(F)nc3)ccnc2C#N)CC1] | 1.2 eq 2-fluoropyridine-5- boronic acid; 4.0 eq. K₂CO₃ solution; 90 min at 100° C. |
| 11 | Int. IV.3 + 2-fluoro- pyridine-5- boronic acid | 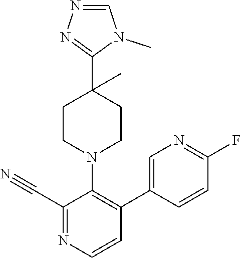 [SMILES: Cn1cnnc1C1(C)CCN(c2c(-c3ccc(F)nc3)ccnc2C#N)CC1] | 1.2 eq 2-fluoropyridine-5- boronic acid; 4.0 eq. K₂CO₃ solution; 90 min at 100° C.; purified by preparative HPLC (XBridge C18, ACN/water gradient containing 0.1% TFA) |
| 12 | Int. III.1 + Int. V.2 | 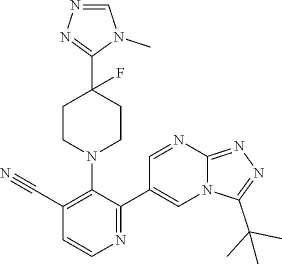 [SMILES: Cn1cnnc1C1(F)CCN(c2c(C#N)ccnc2-c2cnc3nnc(C(C)(C)C)n3c2)CC1] | 4 h at 95° C.; Purification by preparative HPLC (Sunfire C18; ACN/water gradient contain- ing 0.1% TFA) |
| 13 | Int. III.1 + Int. V.3 | 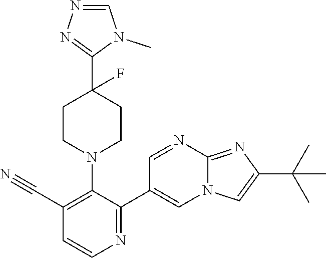 [SMILES: Cn1cnnc1C1(F)CCN(c2c(C#N)ccnc2-c2cnc3nc(C(C)(C)C)cn3c2)CC1] | 1 h at 100° C.; 1.5 eq of Int. V.3; Purification by prepara- tive HPLC (Sunfire C18; ACN/water gradient contain- ing 0.1% TFA; then XBridge C18, ACN/water gradient containing 0.1% NH₃) |
| 14 | Int. III.1 + 3-(4,4,5,5-tetra- methyl-1,3,2-di- oxaboroloan-2- yl)pyrazolo[1,5- b]pyridazine | 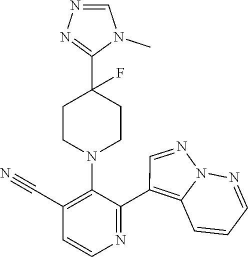 [SMILES: Cn1cnnc1C1(F)CCN(c2c(C#N)ccnc2-c2cnn3ncccc23)CC1] | 1.2 eq. of Int. V.4; 2.5 eq. of a 2 M K₃PO₄ solution instead of K₂CO₃; XPhos Pd G3 (CAS: 1445085-55-1) instead of Pd(dppf)Cl₂; 18 h at 90° C.; purification by preparative HPLC (XBridge C18; ACN/water gradient contain- ing 0.1% TFA) and then fil- tered through Biotage SNAP Cartridge KP-NH with DCM/MeOH 19:1 as eluent |
| 15 | Int. III.1 + Int V.4 | 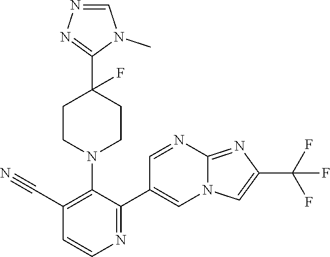 [SMILES: Cn1cnnc1C1(F)CCN(c2c(C#N)ccnc2-c2cnc3nc(C(F)(F)F)cn3c2)CC1] | 1.4 eq Int. V.5; 3.0 eq K₂CO₃ solution; purification by pre- parative HPLC (Sunfire C18; ACN/water gradient contain- ing 0.1% TFA) |
| 16 | Int. III.5 + 2-fluoro- pyridine-5- boronic acid pinacol ester | 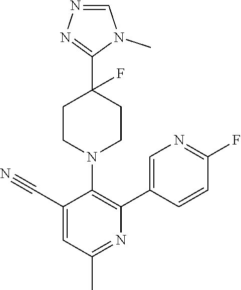 [SMILES: Cc1cc(C#N)c(N2CCC(F)(c3nncn3C)CC2)c(-c2ccc(F)nc2)n1] | 3.0 eq K₂CO₃ solution; 1 h at 100° C.; purification by pre- parative HPLC (Sunfire C18; ACN/water gradient contain- ing 0.1% TFA) and then fil- tered through Biotage SNAP Cartridge KP-NH with DCM/MeOH 19:1 as eluent |
| 17 | Int. III.6 + 2-fluoro- pyridine-5- boronic acid pinacol ester | 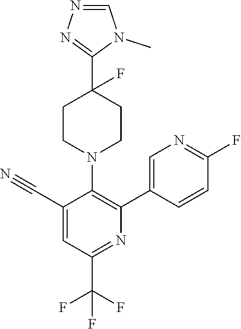 [SMILES: Cn1cnnc1C1(F)CCN(c2c(C#N)cc(C(F)(F)F)nc2-c2ccc(F)nc2)CC1] | 1.2 eq 2-fluoropyridine-5- boronic acid pinacol ester; 2 h at 95° C. |
| 18 | Int. III.4 + 2-fluoro-3- chloro-pyridine- 5-boronic acid | 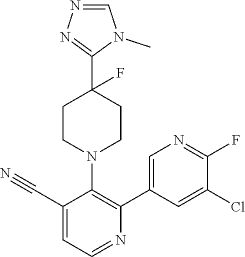 [SMILES: Cn1cnnc1C1(F)CCN(c2c(C#N)ccnc2-c2cnc(F)c(Cl)c2)CC1] | 1.5 eq 2-fluoro-3-chloro-pyr- idine-5-boronic acid; 5 h at 80° C.; purified by prepara- tive HPLC (Sunfire C18, ACN/water gradient contain- ing 0.1% TFA) |
| 19 | Int. III.4 + 5-(1H-pyrazol- 1-yl)pyridine-3- boronic acid pinacol ester | 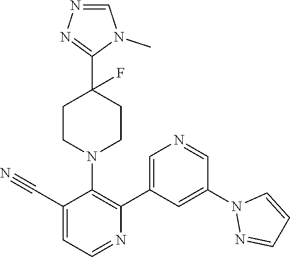 [SMILES: Cn1cnnc1C1(F)CCN(c2c(C#N)ccnc2-c2cncc(-n3cccn3)c2)CC1] | 1.2 eq. of Int. V.4; 2.5 eq. of a 2 M K3PO4 solution instead of K₂CO₃; XPhos Pd G3 (CAS: 1445085-55-1) instead of Pd(dppf)Cl₂; purified by preparative HPLC (Sunfire C18, acetonitrile/water gradi- ent containing 0.1% TFA) |
| 20 | Int. III.4 + 5-cyclo- propylpyridin-3- ylboronic acid pinacol ester | 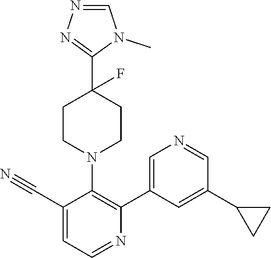 [SMILES: Cn1cnnc1C1(F)CCN(c2c(C#N)ccnc2-c2cncc(C3CC3)c2)CC1] | 1.2 eq. of Int. V.4; 2.5 eq. of a 2 M K₃PO₄ solution instead of K₂CO₃; XPhos Pd G3 (CAS: 1445085-55-1) instead of Pd(dppf)Cl₂; purified by preparative HPLC (Sunfire C18, acetonitrile/water gradi- ent containing 0.1% TFA) |
| 21 | Int. III.4 + 2-methyl-2H- pyrazolo[3,4- b]pyridine-5- boronic acid | 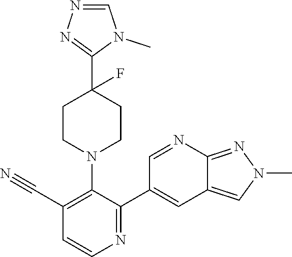 [SMILES: Cn1cc2cc(-c3nccc(C#N)c3N3CCC(F)(c4nncn4C)CC3)cnc2n1] | 1.2 eq. of Int. V.4; 2.5 eq. of a 2 M K₃PO₄ solution instead of K₂CO₃; XPhos Pd G3 (CAS: 1445085-55-1) instead of Pd(dppf)Cl₂; purified by preparative HPLC (Sunfire C18, acetonitrile/water gradi- ent containing 0.1% TFA) |
| 22 | Int. III.4 + 4-(tetramethyl- 1,3,2-dioxa- borolan-2-yl) pyridazine | 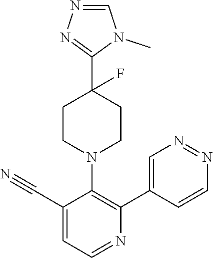 [SMILES: Cn1cnnc1C1(F)CCN(c2c(C#N)ccnc2-c2ccnnc2)CC1] | 1.2 eq. of Int. V.4; 2.5 eq. of a 2 M K₃PO₄ solution instead of K₂CO₃; XPhos Pd G3 (CAS: 1445085-55-1) instead of Pd(dppf)Cl₂; purified by preparative HPLC (Sunfire C18, acetonitrile/water gradi- ent containing 0.1% TFA) |
| 23 | Int. III.4 + 2,3-difluoro- pyridine-5- boronic acid | 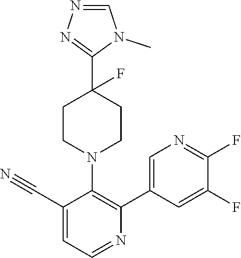 [SMILES: Cn1cnnc1C1(F)CCN(c2c(C#N)ccnc2-c2cnc(F)c(F)c2)CC1] | 1.2 eq. of Int. V.4; 2.5 eq. of a 2 M K₃PO₄ solution instead of K₂CO₃; XPhos Pd G3 (CAS: 1445085-55-1) instead of Pd(dppf)Cl₂; purified by preparative HPLC (Sunfire C18, acetonitrile/water gradi- ent containing 0.1% TFA) |
| 24 | Int. III.4 + 1H-Pyrazole-4- boronic acid | 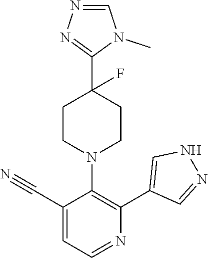 [SMILES: Cn1cnnc1C1(F)CCN(c2c(C#N)ccnc2-c2cn[nH]c2)CC1] | 1.2 eq. of Int. V.4; 2.5 eq. of a 2 M K₃PO₄ solution instead of K₂CO₃; XPhos Pd G3 (CAS: 1445085-55-1) instead of Pd(dppf)Cl₂; purified by preparative HPLC (Sunfire C18, acetonitrile/water gradi- ent containing 0.1% TFA) |
| 25 | Int. III.4 + 4-(tetramethyl- 1,3,2-dioxaboro- lan-2-yl)-1,2- thiazole | 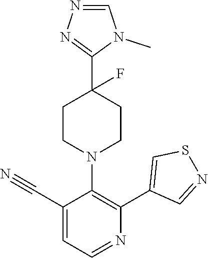 [SMILES: Cn1cnnc1C1(F)CCN(c2c(C#N)ccnc2-c2cnsc2)CC1] | 1.2 eq. of Int. V.4; 2.5 eq. of a 2 M K₃PO₄ solution instead of K₂CO₃; XPhos Pd G3 (CAS: 1445085-55-1) instead of Pd(dppf)Cl₂; purified by preparative HPLC (Sunfire C18, acetonitrile/water gradi- ent containing 0.1% TFA) |

### Analytical Data of Synthesized Examples

| | | Molecular Formula (MW) | |
|---|---|---|---|
| | | ESI-MS | |
| | | HPLC retention time | ¹H NMR (400 MHz, |
| example | Structure | (method) | DMSO-d6): δ in ppm |
| 2 | 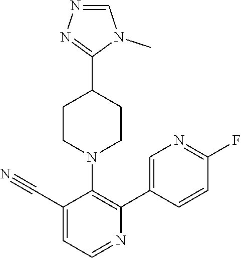 [SMILES: Cn1cnnc1C1CCN(c2c(C#N)ccnc2-c2ccc(F)nc2)CC1] | C₁₉H₁₈FN₇ (M = 363.4 g/mol) ESI-MS: 364 [M + H]⁺ Rt (HPLC): 0.45 min (method G) | 8.46-8.52 (m, 2 H), 8.31 (s, 1 H), 8.23 (td, J = 8.2, 2.5 Hz, 1 H), 7.79 (d, J = 4.8 Hz, 1 H), 7.33 (dd, J = 8.5, 2.7 Hz, 1 H), 3.59 (s, 3 H), 3.24- 3.27 (m, 2 H), 2.99- 3.10 (m, 2 H), 2.87- 2.97 (m, 1 H), 1.65- 1.86 (m, 4 H) |
| 3 | 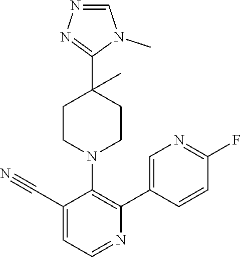 [SMILES: Cn1cnnc1C1(C)CCN(c2c(C#N)ccnc2-c2ccc(F)nc2)CC1] | C₂₀H₂₀FN₇ (M = 377.4 g/mol) ESI-MS: 378 [M + H]⁺ Rt (HPLC): 0.40 min (method C) | 8.45-8.49 (m, 2 H), 8.32 (s, 1 H), 8.21 (td, J = 8.2, 2.5 Hz, 1 H), 7.75 (d, J = 4.9 Hz, 1 H), 7.29 (dd, J = 8.5, 2.5 Hz, 1 H), 3.69 (s, 3 H), 3.01- 3.17 (m, 4 H), 2.16- 2.26 (m, 2 H), 1.62- 1.72 (m, 2 H), 1.29 (s, 3 H) |
| 4 | 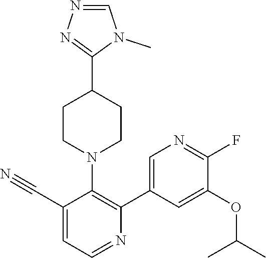 [SMILES: CC(C)Oc1cc(-c2nccc(C#N)c2N2CCC(c3nncn3C)CC2)cnc1F] | C₂₂H₂₄FN₇O (M = 421.5 g/mol) ESI-MS: 422 [M + H]⁺ Rt (HPLC): 0.65 min (method I) | 8.49 (d, J = 4.9 Hz, 1 H), 8.31 (s, 1 H), 7.86-7.93 (m, 2 H), 7.78 (d, J = 4.9 Hz, 1 H), 4.78 (spt, J = 6.0 Hz, 1 H), 3.59 (s, 3 H), 3.31-3.35 (m, 2 H), 2.98-3.12 (m, 2 H), 2.86-2.97 (m, 1 H), 1.66-1.89 (m, 4 H), 1.33 (d, J = 6.0 Hz, 6 H) |
| 5 | 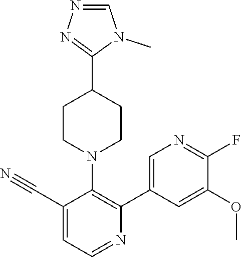 [SMILES: COc1cc(-c2nccc(C#N)c2N2CCC(c3nncn3C)CC2)cnc1F] | C₂₀H₂₀F₂N₇O (M = 393.4 g/mol) ESI-MS: 394 [M + H]⁺ Rt (HPLC): 0.49 min (method J) | 9.17 (s, 1 H), 8.51 (d, J = 4.8 Hz, 1 H), 7.87- 7.96 (m, 2 H), 7.81 (d, J = 4.8 Hz, 1 H), 3.96 (s, 3 H), 3.77 (s, 3 H), 3.31 (br d, J = 12.5 Hz, 2 H), 3.13-3.23 (m, 1 H), 2.99-3.10 (m, 2 H), 1.87-1.95 (m, 2 H), 1.66-1.78 (m, 2 H) |
| 6 | 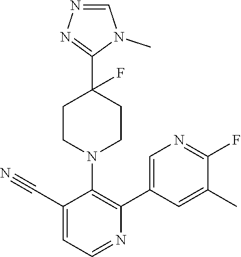 [SMILES: Cc1cc(-c2nccc(C#N)c2N2CCC(F)(c3nncn3C)CC2)cnc1F] | C₂₀H₁₉F₂N₇ (M = 395.4 g/mol) ESI-MS: 396 [M + H]⁺ Rt (HPLC): 0.46 min (method C) | 8.53 (d, J = 4.8 Hz, 1 H), 8.48 (s, 1 H), 8.29 (br s, 1 H), 8.11 (dd, J = 9.7, 1.6 Hz, 1 H), 7.81 (d, J = 4.8 Hz, 1 H), 3.73 (d, J = 1.6 Hz, 3 H), 3.28 (br s, 4 H), 2.31 (s, 3 H), 2.11-2.27 (m, 4 H) |
| 7 | 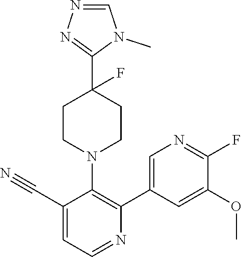 [SMILES: COc1cc(-c2nccc(C#N)c2N2CCC(F)(c3nncn3C)CC2)cnc1F] | C₂₀H₁₉F₂N₇O (M = 411.4 g/mol) ESI-MS: 412 [M + H]⁺ Rt (HPLC): 0.75 min (method G) | 8.55 (s, 1 H), 8.52-8.54 (m, 1 H), 7.96 (d, J = 1.6 Hz, 1 H), 7.89-7.94 (m, 1 H), 7.83 (d, J = 4.8 Hz, 1 H), 3.93 (s, 3 H), 3.74 (d, J = 1.6 Hz, 3 H), 3.17- 3.33 (m, 4 H), 2.13- 2.28 (m, 4 H) |
| 8 | 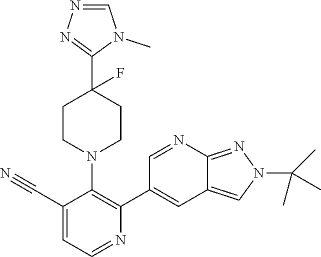 [SMILES: Cn1cnnc1C1(F)CCN(c2c(C#N)ccnc2-c2cnc3nn(C(C)(C)C)cc3c2)CC1] | C₂₄H₂₆FN₉ (M = 459.5 g/mol) ESI-MS: 460 [M + H]⁺ Rt (HPLC): 0.48 min (method B) | 8.83 (d, J = 2.3 Hz, 1 H), 8.64 (s, 1 H), 8.55 (d, J = 4.8 Hz, 1 H), 8.46 (s, 1 H), 8.35 (d, J = 2.2 Hz, 1 H), 7.79 (d, J = 4.8 Hz, 1 H), 3.70 (d, J = 1.5 Hz, 3 H), 3.20-3.27 (m, 4 H), 2.14-2.34 (m, 4 H), 1.72 (s, 9 H) |
| 9 | 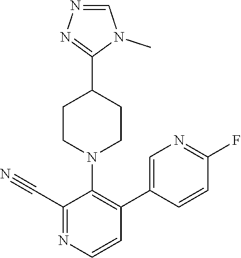 [SMILES: Cn1cnnc1C1CCN(c2c(-c3ccc(F)nc3)ccnc2C#N)CC1] | C₁₉H₁₈FN₇ (M = 363.4 g/mol) ESI-MS: 364 [M + H]⁺ Rt (HPLC): 0.41 min (method B) | 8.45 (d, J = 4.8 Hz, 1 H), 8.38 (d, J = 2.3 Hz, 1 H), 8.30 (s, 1 H), 8.15 (td, J = 8.2, 2.5 Hz, 1 H), 7.60 (d, J = 4.7 Hz, 1 H), 7.38 (dd, J = 8.5, 2.7 Hz, 1 H), 3.58 (s, 3 H), 3.18- 3.27 (m, 2 H), 2.93- 3.03 (m, 2 H), 2.81- 2.93 (m, 1 H), 1.61- 1.86 (m, 4 H) |
| 10 | 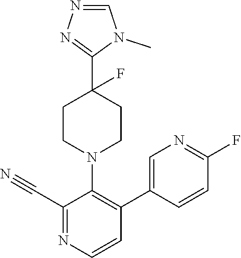 [SMILES: Cn1cnnc1C1(F)CCN(c2c(-c3ccc(F)nc3)ccnc2C#N)CC1] | C₁₉H₁₇F₂N₇ (M = 377.4 g/mol) ESI-MS: 382 [M + H]⁺ Rt (HPLC): 0.45 min (method C) | 8.49 (d, J = 4.7 Hz, 1 H), 8.46-8.48 (m, 1 H), 8.40 (d, J = 2.4 Hz, 1 H), 8.16 (td, J = 8.2, 2.5 Hz, 1 H), 7.63 (d, J = 4.7 Hz, 1 H), 7.38 (dd, J = 8.4, 2.6 Hz, 1 H), 3.73 (d, J = 1.6 Hz, 3 H), 3.16 (br s, 4 H), 2.08-2.25 (m, 4 H) |
| 11 | 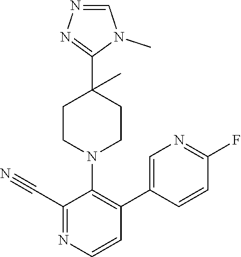 [SMILES: Cn1cnnc1C1(C)CCN(c2c(-c3ccc(F)nc3)ccnc2C#N)CC1] | C₂₀H₂₀FN₇ (M = 377.4 g/mol) ESI-MS: 378 [M + H]⁺ Rt (HPLC): 0.45 min (method B) | 8.97 (s, 1 H), 8.46 (d, J = 4.7 Hz, 1 H), 8.36 (d, J = 2.4 Hz, 1 H), 8.12 (td, J = 8.2, 2.5 Hz, 1 H), 7.60 (d, J = 4.8 Hz, 1 H), 7.33 (dd, J = 8.5, 2.5 Hz, 1 H), 3.81 (s, 3 H), 2.96- 3.15 (m, 4 H), 2.10- 2.21 (m, 2 H), 1.65- 1.78 (m, 2 H), 1.33 (s, 3H) |
| 12 | 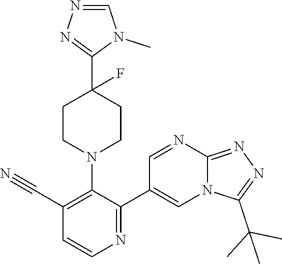 [SMILES: Cn1cnnc1C1(F)CCN(c2c(C#N)ccnc2-c2cnc3nnc(C(C)(C)C)n3c2)CC1] | C₂₃H₂₅FN₁₀ (M = 460.5 g/mol) ESI-MS: 461 [M + H]⁺ Rt (HPLC): 0.66 min (method G) | 9.55 (d, J = 2.3 Hz, 1 H), 9.16 (d, J = 2.3 Hz, 1 H), 8.61 (d, J = 4.8 Hz, 1 H), 8.54 (s, 1 H), 7.89 (d, J = 4.8 Hz, 1 H), 3.74 (d, J = 1.4 Hz, 3 H), 3.26- 3.44 (m, 4 H), 2.09- 2.29 (m, 4 H), 1.44 (s, 9 H) |
| 13 | 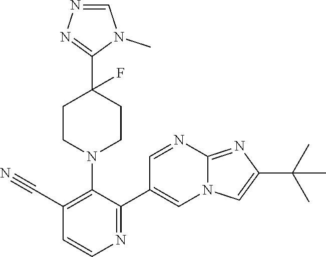 [SMILES: Cn1cnnc1C1(F)CCN(c2c(C#N)ccnc2-c2cnc3nc(C(C)(C)C)cn3c2)CC1] | C₂₄H₂₆FN₉ (M = 459.5 g/mol) ESI-MS: 460 [M + H]⁺ Rt (HPLC): 0.36 min (method C) | 9.17 (d, J = 2.4 Hz, 1 H), 8.80 (d, J = 2.4 Hz, 1 H), 8.57 (d, J = 4.8 Hz, 1 H), 8.48 (s, 1 H), 7.84 (d, J = 4.8 Hz, 1 H), 7.78 (s, 1 H), 3.73 (d, J = 1.4 Hz, 3 H), 3.26-3.43 (m, 4 H), 2.19-2.37 (m, 4 H), 1.35 (s, 9 H) |
| 14 | 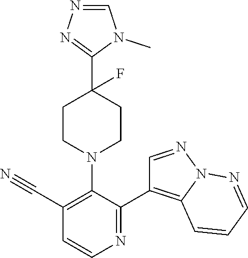 [SMILES: Cn1cnnc1C1(F)CCN(c2c(C#N)ccnc2-c2cnn3ncccc23)CC1] | C₂₀H₁₈FN₉ (M = 403.4 g/mol) ESI-MS: 404 [M + H]⁺ Rt (HPLC): 0.82 min (method K) | 8.74 (s, 1H), 8.62 (dd, J = 1.9, 9.1 Hz, 1H), 8.59- 8.54 (m, 2H), 8.51 (s, 1H), 7.69 (d, J = 4.9 Hz, 1H), 7.36 (dd, J = 4.4, 9.1 Hz, 1H), 3.76 (d, J = 1.5 Hz, 3H), 3.56-3.46 (m, 2H), 3.28-3.21 (m, 2H), 2.45-2.25 (m, 4H) |
| 15 | 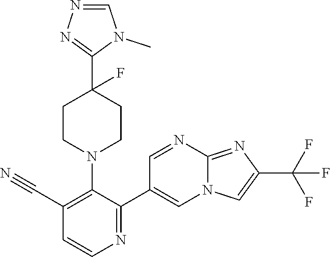 [SMILES: Cn1cnnc1C1(F)CCN(c2c(C#N)ccnc2-c2cnc3nc(C(F)(F)F)cn3c2)CC1] | C₂₁H₁₇F₄N₉ (M = 471.4 g/mol) ESI-MS: 472 [M + H]⁺ Rt (HPLC): 0.42 min (method C) | 9.32 (d, J = 2.4 Hz, 1 H), 9.10 (d, J = 2.3 Hz, 1 H), 8.59-8.63 (m, 2 H), 8.55 (s, 1 H), 7.89 (d, J = 4.9 Hz, 1 H), 3.74 (d, J = 1.3 Hz, 3 H), 3.36- 3.46 (m, 2 H), 3.27- 3.36 (m, 2 H), 2.14- 2.34 (m, 4 H) |
| 16 | 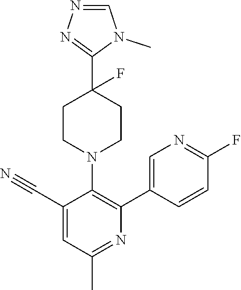 [SMILES: Cc1cc(C#N)c(N2CCC(F)(c3nncn3C)CC2)c(-c2ccc(F)nc2)n1] | C₂₀H₁₉F₂N₇ (M = 395.4 g/mol) ESI-MS: 396 [M + H]⁺ Rt (HPLC): 0.64 min (method L) | 8.50-8.45 (m, 2H), 8.22 (dt, J = 2.4, 8.2 Hz, 1H), 7.72 (s, 1H), 7.32 (dd, J = 2.6, 8.4 Hz, 1H), 3.73 (d, J = 1.5 Hz, 3H), 3.28-3.19 (m, 2H), 3.16-3.08 (m, 2H), 2.51 (s, 3H), 2.25- 2.16 (m, 3H), 2.16- 2.07 (m, 1H) |
| 17 | 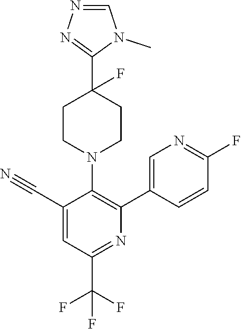 [SMILES: Cn1cnnc1C1(F)CCN(c2c(C#N)cc(C(F)(F)F)nc2-c2ccc(F)nc2)CC1] | C₂₀H₁₆F₅N₇ (M = 449.4 g/mol) ESI-MS: 450 [M + H]⁺ Rt (HPLC): 0.55 min (method C) | 8.52 (d, J = 2.3 Hz, 1 H), 8.49 (s, 1 H), 8.39 (s, 1 H), 8.26 (td, J = 8.2, 2.5 Hz, 1 H), 7.38 (dd, J = 8.5, 2.5 Hz, 1 H), 3.73 (d, J = 1.5 Hz, 3 H), 3.22-3.38 (m, 4 H), 2.13-2.30 (m, 4 H) |
| 18 | 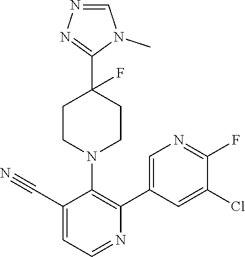 [SMILES: Cn1cnnc1C1(F)CCN(c2c(C#N)ccnc2-c2cnc(F)c(Cl)c2)CC1] | C₁₉H₁₆ClF₂N₇ (M = 415.8 g/mol) ESI-MS: 416/418 [M + H]⁺ Rt (HPLC): 0.77 min (method G) | 8.60 (s, 1 H), 8.56 (d, J = 4.8 Hz, 1 H), 8.45- 8.50 (m, 2 H), 7.86 (d, J = 4.8 Hz, 1 H), 3.76 (d, J = 1.1 Hz, 3 H), 3.18- 3.35 (m, 4 H), 2.07- 2.28 (m, 4 H) |
| 19 | 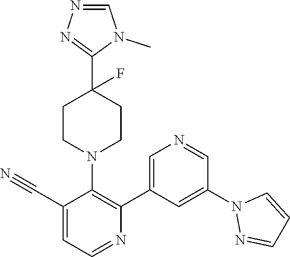 [SMILES: Cn1cnnc1C1(F)CCN(c2c(C#N)ccnc2-c2cncc(-n3cccn3)c2)CC1] | C₂₂H₂₀FN₉ (M = 429.5 g/mol) ESI-MS: 430 [M + H]⁺ Rt (HPLC): 0.55 min (method D) | 9.20 (d, J = 2.5 Hz, 1 H), 8.76 (d, J = 1.9 Hz, 1 H), 8.67 (d, J = 2.5 Hz, 1 H), 8.63 (s, 1 H), 8.58 (d, J = 4.8 Hz, 1 H), 8.54 (t, J = 2.2 Hz, 1 H), 7.86 (d, J = 4.8 Hz, 1 H), 7.83 (d, J = 1.6 Hz, 1 H), 6.63 (dd, J = 2.3, 1.8 Hz, 1 H), 3.73 (d, J = 1.5 Hz, 3 H), 3.23-3.31 (m, 4 H), 2.15-2.31 (m, 4 H) |
| 20 | 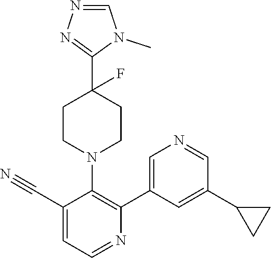 [SMILES: Cn1cnnc1C1(F)CCN(c2c(C#N)ccnc2-c2cncc(C3CC3)c2)CC1] | C₂₂H₂₂FN₇ (M = 403.5 g/mol) ESI-MS: 404 [M + H]⁺ Rt (HPLC): 0.45 min (method L) | 8.78 (d, J = 1.5 Hz, 1 H), 8.68 (d, J = 2.0 Hz, 1 H), 8.59 (s, 1 H), 8.57 (d, J = 4.8 Hz, 1 H), 8.00- 8.05 (m, 1 H), 7.87 (d, J = 4.9 Hz, 1 H), 3.75 (d, J = 1.5 Hz, 3 H), 3.14- 3.30 (m, 4 H), 2.10- 2.27 (m, 5 H), 1.06- 1.14 (m, 2 H), 0.86- 0.92 (m, 2 H) |
| 21 | 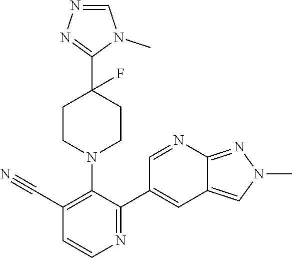 [SMILES: Cn1cc2cc(-c3nccc(C#N)c3N3CCC(F)(c4nncn4C)CC3)cnc2n1] | C₂₁H₂₀FN₉ (M = 417.5 g/mol) ESI-MS: 418 [M + H]⁺ Rt (HPLC): 0.46 min (method L) | 8.78-8.89 (m, 1 H), 8.52-8.62 (m, 3 H), 8.43 (d, J = 1.9 Hz, 1 H), 7.79 (br d, J = 4.8 Hz, 1 H), 4.24 (s, 3 H), 3.72 (s, 3 H), 3.14-3.31 (m, 4 H), 2.07-2.27 (m, 4 H) |
| 22 | 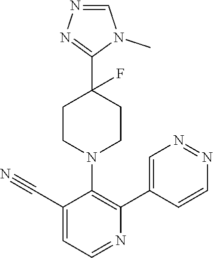 [SMILES: Cn1cnnc1C1(F)CCN(c2c(C#N)ccnc2-c2ccnnc2)CC1] | C₁₈H₁₇FN₈ (M = 364.4 g/mol) ESI-MS: 365 [M + H]⁺ Rt (HPLC): 0.45 min (method L) | 9.50 (dd, J = 2.3, 1.3 Hz, 1 H), 9.40 (dd, J = 5.3, 1.2 Hz, 1 H), 8.60 (d, J = 4.8 Hz, 1 H), 8.55 (s, 1 H), 7.90-7.94 (m, 2 H), 3.74 (br d, J = 1.6 Hz, 3 H), 3.18-3.34 (m, 4 H), 2.09-2.28 (m, 4 H) |
| 23 | 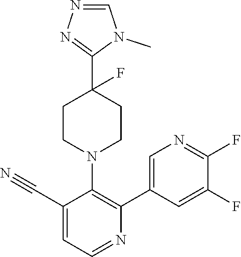 [SMILES: Cn1cnnc1C1(F)CCN(c2c(C#N)ccnc2-c2cnc(F)c(F)c2)CC1] | C₁₉H₁₆F₃N₇ (M = 399.4 g/mol) ESI-MS: 400 [M + H]⁺ Rt (HPLC): 0.65 min (method L) | 8.57 (s, 1 H), 8.55 (d, J = 4.9 Hz, 1 H), 8.28- 8.35 (m, 2 H), 7.86 (d, J = 4.8 Hz, 1 H), 3.75 (d, J = 1.6 Hz, 3 H), 3.17- 3.34 (m, 4 H), 2.09- 2.29 (m, 4 H) |
| 24 | 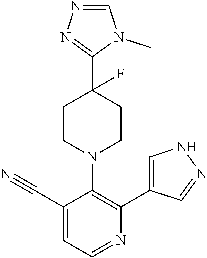 [SMILES: Cn1cnnc1C1(F)CCN(c2c(C#N)ccnc2-c2cn[nH]c2)CC1] | C₁₇H₁₇FN₈ (M = 352.4 g/mol) ESI-MS: 353 [M + H]⁺ Rt (HPLC): 0.48 min (method D) | 8.56 (s, 1 H), 8.50 (d, J = 4.8 Hz, 1 H), 8.24 (s, 2 H), 7.60 (d, J = 4.8 Hz, 1 H), 3.80 (d, J = 1.6 Hz, 3 H), 3.51-3.55 (m, 2 H), 3.12-3.20 (m, 2 H), 2.35-2.45 (m, 4 H) |
| 25 | 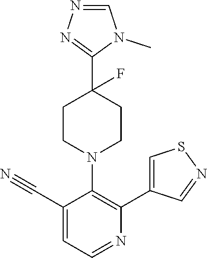 [SMILES: Cn1cnnc1C1(F)CCN(c2c(C#N)ccnc2-c2cnsc2)CC1] | C₁₇H₁₆FN₇S (M = 369.4 g/mol) ESI-MS: 370 [M + H]⁺ Rt (HPLC): 0.52 min (method D) | 9.48 (s, 1 H), 8.97 (s, 1 H), 8.64 (s, 1 H), 8.54 (d, J = 4.9 Hz, 1 H), 7.78 (d, J = 4.9 Hz, 1 H), 3.78 (d, J = 1.5 Hz, 3 H), 3.34- 3.47 (m, 2 H), 3.14- 3.24 (m, 2 H), 2.24- 2.43 (m, 4 H) |

### Example 26

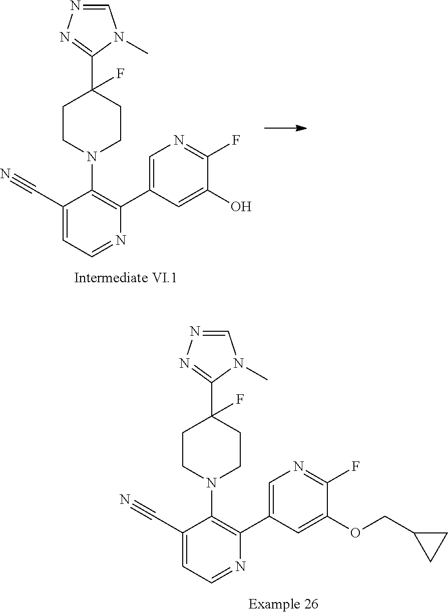 [SMILES: Cn1cnnc1C1(F)CCN(c2c(C#N)ccnc2-c2cnc(F)c(O)c2)CC1 ; Cn1cnnc1C1(F)CCN(c2c(C#N)ccnc2-c2cnc(F)c(OCC3CC3)c2)CC1]

The intermediate VI.1 (90.0 mg, 0.207 mmol) and cesium carbonate (135.2 mg, 0.414 mmol) are added to DMF (2.0 mL) and (iodomethyl)cyclopropane (56.6 mg, 0.311 mmol) is added. The resulting reaction mixture is heated to 80° C. and stirred at this temperature for 8 h. After cooling to ambient temperature, it is diluted with a mixture of ACN/water/TFA and purified by preparative HPLC (XBridge C18, ACN/water gradient containing 0.1% TFA) to yield the desired product.

C₂₃H₂₃F₂N₇O (M=451.5 g/mol)

ESI-MS: 452 [M+H]⁺

Rt (HPLC): 0.52 min (method C)

¹H NMR (400 MHz, DMSO-d6) δ ppm 8.53 (s, 1H), 8.51-8.53 (m, 1H), 8.52 (d, J=4.8 Hz, 1H), 7.94 (t, J=1.6 Hz, 1H), 7.88 (dd, J=10.1, 1.8 Hz, 1H), 7.82 (d, J=4.9 Hz, 1H), 3.99 (d, J=7.1 Hz, 2H), 3.74 (d, J=1.6 Hz, 3H), 3.15-3.32 (m, 4H), 2.09-2.30 (m, 4H), 1.20-1.33 (m, 1H), 0.52-0.65 (m, 1H), 0.30-0.42 (m, 2H).

### Examples Synthesized Analogous to the Procedure Described for Example 26

| | Starting | | Deviation from general |
|---|---|---|---|
| example | materials | Structure | procedure |
| 27 | Int. VI + Iodocyclo- propane | 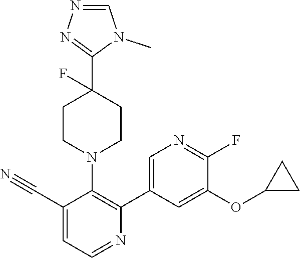 [SMILES: Cn1cnnc1C1(F)CCN(c2c(C#N)ccnc2-c2cnc(F)c(OC3CC3)c2)CC1] | 2.0 eq. iodocyclopropane, 3.5 eq. cesium carbonate, 12 h at 130° C. After initial purifica- tion as described, purified by chiral preparative SFC (CHI- RAL ART ® Cellulose-SZ 10 x 250 mm, 5 μm; CO₂/MeOH = 3:1, 40° C., 15 mL/min) |
| 28 | Int. VI + iodocyclo- propane | 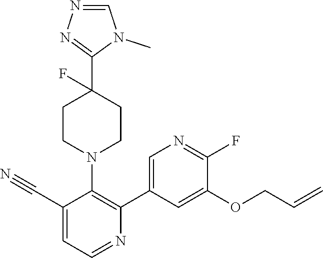 [SMILES: C=CCOc1cc(-c2nccc(C#N)c2N2CCC(F)(c3nncn3C)CC2)cnc1F] | Obtained as a by-product dur- ing the synthesis of example 27. After initial purification as described, purified by chiral preparative SFC as described for example 27. |

### Analytical Data of Synthesized Examples

| | | Molecular Formula | |
|---|---|---|---|
| | | (MW) | |
| | | ESI-MS | |
| | | HPLC retention time | ¹H NMR (400 MHz, |
| example | Structure | (method) | DMSO-d6): δ in ppm |
| 27 | 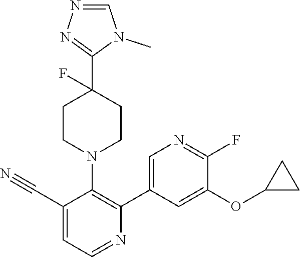 [SMILES: Cn1cnnc1C1(F)CCN(c2c(C#N)ccnc2-c2cnc(F)c(OC3CC3)c2)CC1] | C₂₂H₂₁F₂N₇O (M = 437.4 g/mol) ESI-MS: 438 [M + H]⁺ Rt (HPLC): 6.36 min (method M) | 8.54 (d, J = 4.8 Hz, 1 H), 8.49 (s, 1 H), 8.09 (dd, J = 9.9, 2.0 Hz, 1 H), 8.00 (t, J = 1.6 Hz, 1 H), 7.84 (d, J = 4.8 Hz, 1 H), 4.05 (tt, J = 5.8, 3.0 Hz, 1 H), 3.74 (d, J = 1.5 Hz, 3 H), 3.18- 3.29 (m, 4 H), 2.14-2.31 (m, 4 H), 0.73-0.89 (m, 4 H) |
| 28 | 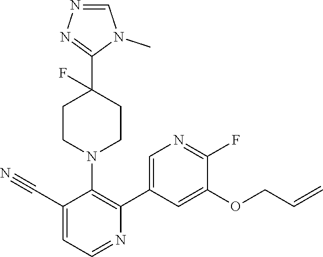 [SMILES: C=CCOc1cc(-c2nccc(C#N)c2N2CCC(F)(c3nncn3C)CC2)cnc1F] | C₂₂H₂₁F₂N₇O (M = 437.4 g/mol) ESI-MS: 438 [M + H]⁺ Rt (HPLC): 5.38 min (method M) | 8.53 (d, J = 4.9 Hz, 1 H), 8.48 (s, 1 H), 7.96 (t, J = 1.5 Hz, 1 H), 7.91 (dd, J = 10.0, 1.8 Hz, 1 H), 7.82 (d, J = 4.8 Hz, 1 H), 6.01-6.13 (m, 1 H), 5.42- 5.50 (m, 1 H), 5.28-5.35 (m, 1 H), 4.71-4.76 (m, 2 H), 3.73 (d, J = 1.5 Hz, 3 H), 3.16-3.27 (m, 4 H), 2.11- 2.28 (m, 4 H) |

### Example 29

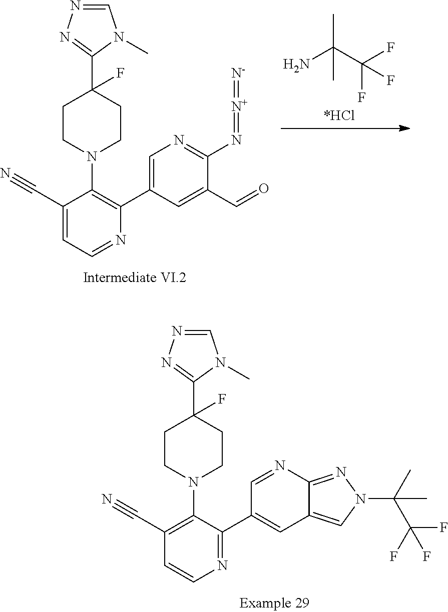

Under an argon atmosphere, intermediate V1.2 (20.0 mg, 0.046 mmol) and iron(II) bromide (2.0 mg, 9.5 μmol) are suspended in DMSO (1.0 mL). 1,1,1-Trifluoro-2-methyl-propan-2-amine hydrochloride (11.3 mg, 0.069 mmol) is added, and the resulting reaction mixture is heated to 120° C. and stirred at this temperature for 2 h. After cooling to ambient temperature, the mixture is diluted with water and purified by preparative HPLC (ZORBAX StableBond C18, water/ACN gradient containing 0.1% TFA) to yield the desired product.

C₂₄H₂₃F₄N₉ (M=513.5 g/mol)

ESI-MS: 514 [M+H]⁺

Rt (HPLC): 0.65 m (method L)

¹H NMR (400 MHz, DMSO-d6) δ ppm 8.95 (s, 1H), 8.92 (d, J=2.2 Hz, 1H), 8.56 (d, J=4.8 Hz, 1H), 8.49 (s, 1H), 8.41 (d, J=2.2 Hz, 1H), 7.81 (d, J=4.8 Hz, 1H), 3.71 (d, J=1.3 Hz, 3H), 3.23-3.28 (m, 4H), 2.15-2.30 (m, 4H), 2.03 (s, 6H).

### Examples Synthesized Analogous to the Procedure Described for Example 29

| exam- | Starting | | Deviation from general |
|---|---|---|---|
| ple | materials | Structure | procedure |
| 30 | Int. VI.2 + 1-(trifluoro- methyl)cyclo- propanamine hydrochloride | 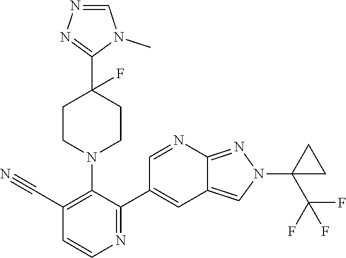 [SMILES: Cn1cnnc1C1(F)CCN(c2c(C#N)ccnc2-c2cnc3nn(C4(C(F)(F)F)CC4)cc3c2)CC1] | 11 h at 120° C. |
| 31 | Int. VI.2 + 3,3-difluoro-1- methylcyclo- butanamine hydrochloride | 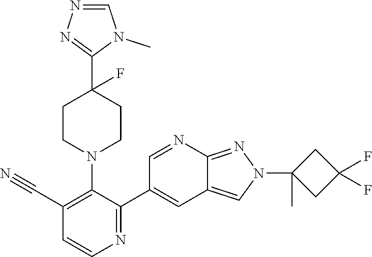 [SMILES: Cn1cnnc1C1(F)CCN(c2c(C#N)ccnc2-c2cnc3nn(C4(C)CC(F)(F)C4)cc3c2)CC1] | — |
| 32 | Int. VI.2 + 2-(4-fluoro- phenyl)pro- pan-2-amine | 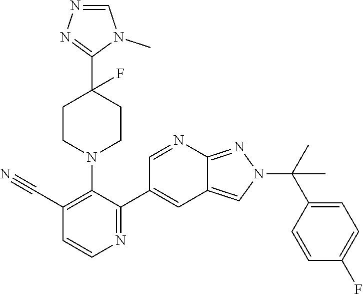 [SMILES: Cn1cnnc1C1(F)CCN(c2c(C#N)ccnc2-c2cnc3nn(C(C)(C)c4ccc(F)cc4)cc3c2)CC1] | — |

### Analytical Data of Synthesized Examples

| | | Molecular Formula | |
|---|---|---|---|
| | | (MW) | |
| | | ESI-MS | |
| | | HPLC retention time | ¹H NMR (400 MHz, |
| example | Structure | (method) | DMSO-d6): δ in ppm |
| 30 | 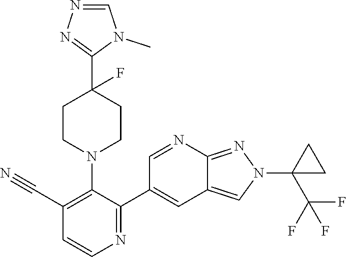 [SMILES: Cn1cnnc1C1(F)CCN(c2c(C#N)ccnc2-c2cnc3nn(C4(C(F)(F)F)CC4)cc3c2)CC1] | C₂₄H₂₁F₄N₉ (M = 511.5 g/mol) ESI-MS: 512 [M + H]⁺ Rt (HPLC): 0.77 min (method G) | 8.94 (d, J = 2.3 Hz, 1 H), 8.86 (s, 1 H), 8.56 (d, J = 4.8 Hz, 1 H), 8.50-8.54 (m, 1 H), 8.41 (d, J = 2.2 Hz, 1 H), 7.82 (d, J = 4.8 Hz, 1 H), 3.72 (d, J = 1.0 Hz, 3 H), 3.20-3.32 (m, 4 H), 2.13- 2.31 (m, 4 H), 1.83-1.90 (m, 2 H), 1.77-1.83 (m, 2 H) |
| 31 | 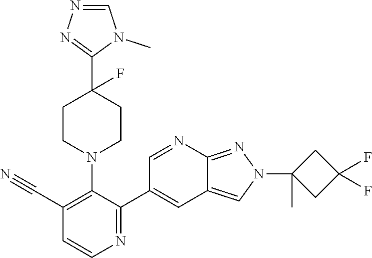 [SMILES: Cn1cnnc1C1(F)CCN(c2c(C#N)ccnc2-c2cnc3nn(C4(C)CC(F)(F)C4)cc3c2)CC1] | C₂₅H₂₄F₃N₉ (M = 507.5 g/mol) ESI-MS: 508 [M + H]⁺ Rt (HPLC): 0.78 min (method G) | 8.89 (d, J = 2.2 Hz, 1 H), 8.78 (s, 1 H), 8.54-8.58 (m, 2 H), 8.40 (d, J = 2.2 Hz, 1 H), 7.80 (d, J = 4.8 Hz, 1 H), 3.72 (d, J = 1.4 Hz, 3 H), 3.53-3.67 (m, 2 H), 3.21-3.30 (m, 4 H), 3.07-3.19 (m, 2 H), 2.13- 2.31 (m, 4 H), 1.92 (s, 3 H) |
| 32 | 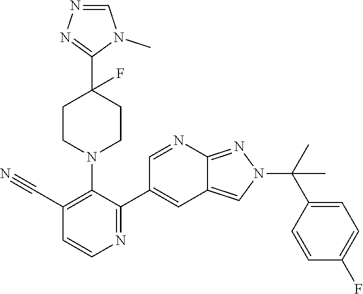 [SMILES: Cn1cnnc1C1(F)CCN(c2c(C#N)ccnc2-c2cnc3nn(C(C)(C)c4ccc(F)cc4)cc3c2)CC1] | C₂₉H₂₇F₂N₉ (M = 539.6 g/mol) ESI-MS: 540 [M + H]⁺ Rt (HPLC): 0.71 min (method I) | 8.84 (d, J = 2.2 Hz, 1 H), 8.67 (s, 1 H), 8.55 (d, J = 4.9 Hz, 1 H), 8.47 (s, 1 H), 8.38 (d, J = 2.3 Hz, 1 H), 7.79 (d, J = 4.8 Hz, 1 H), 7.11-7.22 (m, 4 H), 3.71 (d, J = 1.4 Hz, 3 H), 3.21-3.28 (m, 4 H), 2.20-2.31 (m, 2 H), 2.14-2.20 (m, 2 H), 2.12 (s, 6 H) |

### Analytical HPLC Methods

Method A

| time | Vol % water | Vol % ACN | Flow |
|---|---|---|---|
| (min) | (incl. 0.04% TFA) | (incl. 0.02% TFA) | [mL/min] |
| 0.00 | 95 | 5 | 1.5 |
| 0.70 | 5 | 95 | 1.5 |
| 1.16 | 5 | 95 | 1.5 |
| 1.50 | 95 | 5 | 1.5 |

Analytical column: Kinetex EVO C18_2.1×30 mm_5 μm; column temperature: 40° C.

Method B

| time | Vol % water | Vol % | Flow |
|---|---|---|---|
| (min) | (incl. 0.1% NH₃) | ACN | [mL/min] |
| 0.00 | 95 | 5 | 1.3 |
| 0.02 | 95 | 5 | 1.3 |
| 1.00 | 0 | 100 | 1.3 |
| 1.30 | 0 | 100 | 1.3 |

Device description: Waters Acquity; Analytical column: XBridge (Waters) BEH C18_2.1×30 mm_2.5 μm; column temperature: 60° C.

Method C

| time | Vol % water | Vol % | Flow |
|---|---|---|---|
| (min) | (incl. 0.1% TFA) | ACN | [mL/min] |
| 0.00 | 99 | 1 | 1.6 |
| 0.02 | 99 | 1 | 1.6 |
| 1.00 | 0 | 100 | 1.6 |
| 1.10 | 0 | 100 | 1.6 |

Device description: Waters Acquity; Analytical column: Xbridge (Waters) BEH C18_2.1×30 mm_1.7 μm; column temperature: 60° C.

Method D

| time | Vol % water | Vol % | Flow |
|---|---|---|---|
| (min) | (incl. 0.1% NH₃) | ACN | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity; Analytical column: Xbridge (Waters) C18_3.0×30 mm_2.5 μm; column temperature: 60° C.

Method E

| time | Vol % water | Vol % | Flow |
|---|---|---|---|
| (min) | (incl. 0.1% NH₃) | ACN | [mL/min] |
| 0.00 | 95 | 5 | 1.3 |
| 0.02 | 95 | 5 | 1.3 |
| 1.00 | 0 | 100 | 1.3 |
| 1.30 | 0 | 100 | 1.3 |

Device description: Waters Acquity; Analytical column: Xbridge (Waters) BEH Phenyl_2.1×30 mm_1.7 μm; column temperature: 60° C.

Method F

| time | Vol % water | Vol % | Flow |
|---|---|---|---|
| (min) | (incl. 0.1% FA) | ACN | [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200; Analytical column: XBridge (Waters) C18_3.0×30 mm_2.5 μm; column temperature: 60° C.

Method G

| time | Vol % water | Vol % | Flow |
|---|---|---|---|
| (min) | (incl. 0.1% TFA) | ACN | [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200; Analytical column: Zorbax (Agilent) StableBond C18_3.0×30 mm_1.8 μm; column temperature: 60° C.

Method H

| time | Vol % water | Vol % | Flow |
|---|---|---|---|
| (min) | (incl. 0.1% TFA) | ACN | [mL/min] |
| 0.00 | 95 | 5 | 1.5 |
| 1.30 | 0 | 100 | 1.5 |
| 1.50 | 0 | 100 | 1.5 |

Device description: Waters Acquity; Analytical column: Sunfire (Waters) C18_3.0×30 mm_2.5 μm; column temperature: 60° C.

Method I

| time | Vol.% water | Vol % | Flow |
|---|---|---|---|
| (min) | (incl. 0.1% NH₃) | ACN | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity; Analytical column: XBridge (Waters) C18_3.0×30 mm_2.5 μm; column temperature: 60° C.

Method J

| time | Vol.% water | Vol % ACN | Flow |
|---|---|---|---|
| (min) | (incl. 0.1% TFA) | (incl. 0.1% FA) | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity; Analytical column: Sunfire (Waters) C18_3.0×30 mm_2.5 μm; column temperature: 60° C.

Method K

| time | Vol % water | Vol % | Flow |
|---|---|---|---|
| (min) | (incl. 0.1% NH₃) | ACN | [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200; Analytical column: Xbridge (Waters) C18_3.0×30 mm_2.5 μm; column temperature: 60° C.

Method L

| time | Vol % water | Vol % ACN (incl. | Flow |
|---|---|---|---|
| (min) | (incl. 0.1% TFA) | 0.08% TFA) | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity; Analytical column: Sunfire (Waters) C18_3.0×30 mm_2.5 μm; column temperature: 60° C.

Method M

| | | Vol % | |
|---|---|---|---|
| time | Vol % | MeOH, | Flow |
| (min) | scCO₂ | 20 mM NH₃ | [mL/min] |
| 0.00 | 95 | 5 | 4.0 |
| 10.0 | 95 | 5 | 4.0 |

Device description: Agilent 1260 Infinity II SFC; Column: CHIRAL ART (YMC) Cellulose SC_4.6×250 mm_1.7 μm; column temperature: 40° C.

1. A compound of formula (I) 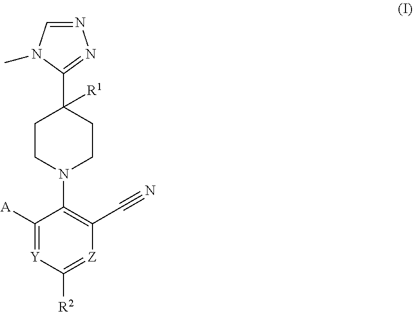
wherein
Y is N and Z is R⁴C or Y is HC and Z is N;
A is A1a which is a 5- or 6-membered mono-heteroaryl ring containing one nitrogen or containing two heteroatom members wherein one is nitrogen and the second is selected from nitrogen or sulphur;
or A is A1b which is a 9- or 10-membered fused bicyclic-heteroaryl ring containing one to four nitrogens; and wherein A1a or A1b is independently substituted with one or two R³;
R¹ is selected from the group R1a, consisting of H, C₁₋₄-alkyl and halo;
R² is selected from the group R2a, consisting of halo, H, C₁₋₄-alkyl, C₃₋₄-cycloalkyl and F₁₋₉-fluoro-C₁₋₄-alkyl;
R³ is selected from the group R3a, consisting of H, halo, C₁₋₄-alkyl, C₃₋₄-cycloalkyl, C₃₋₄-fluorocycloalkyl, F₁₋₉-fluoro-C₁₋₄-alkyl, C₁₋₄-alkyloxy, C₃₋₄-cycloalkyloxy and pyrazolyl, 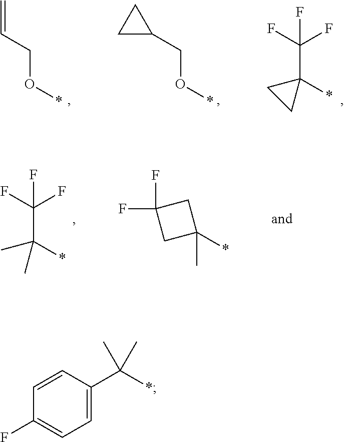
R⁴ is selected from the group R4a, consisting of halo, H, C₁₋₄-alkyl, C₃₋₄-cycloalkyl and F₁₋₉-fluoro-C₁₋₄-alkyl;
or a salt thereof.

2. The compound of formula (I) according to claim 1, wherein A is selected from the group A3 consisting of pyridinyl, pyrazinyl, pyrazolyl, isothiazolyl, imidazo[1,2-a]pyrimidyl, pyrazolo[3,4-b]pyridinyl, [1,2,4]triazolo[4,3-a]pyrimidyl, pyrazolo[1,5-b]pyridazinyl, [1,2,4]triazolo[1,5-a]pyrimidinyl, 2H-[1,2,3]triazolo[4,5-b]pyridinyl, 1H-[1,2,3]triazolo[4,5-b]pyridinyl and 1H-imidazo[4,5-b]pyridinyl and wherein A3 is independently substituted with one or two R³;
or a salt thereof.

3. The compound of formula (I) according to claim 1, wherein A is selected from the group A9 consisting of 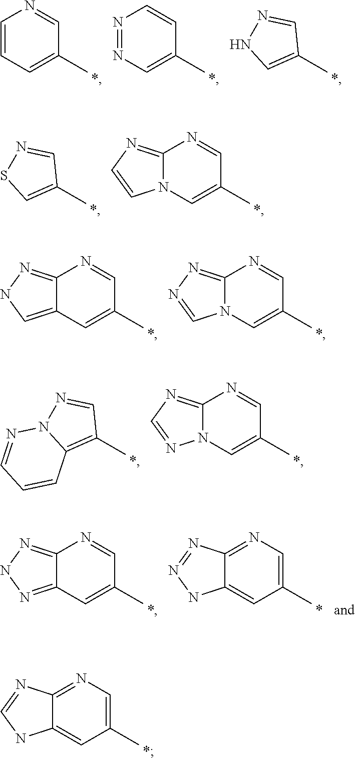
wherein A9 is independently substituted with one or two R³;
or a salt thereof.

4. The compound of formula (I) according to claim 1, wherein R² is selected from the group R2b, consisting of H, F, Cl, C₁₋₄-alkyl and F₁₋₉-fluoro-C₁₋₄-alkyl;
or a salt thereof.

5. The compound of formula (I) according to claim 1, wherein R³ is selected from the group R3b, consisting of H, halo, C₁₋₄-alkyl, C₃₋₄-cycloalkyl, F₁₋₉-fluoro-C₁₋₄-alkyl, C₁₋₄-alkyloxy, C₃₋₄-cycloalkyloxy and pyrazolyl, 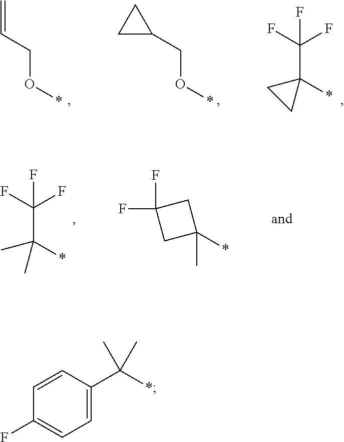
or a salt thereof.

6. The compound of formula (I) according to claim 1, having formula (I-a) 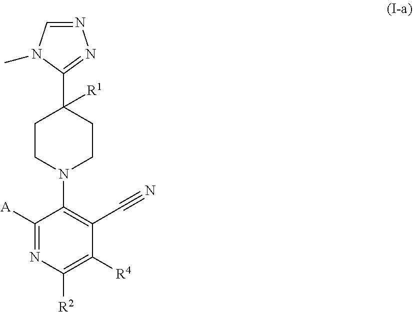
or a salt thereof.

7. The compound of formula (I) according to claim 1, having formula (I-b) 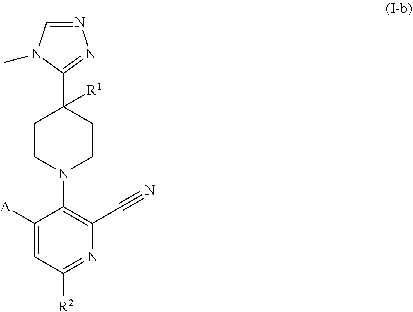
or a salt thereof.

8. The compound of formula (I) according to claim 1, selected from the group consisting of 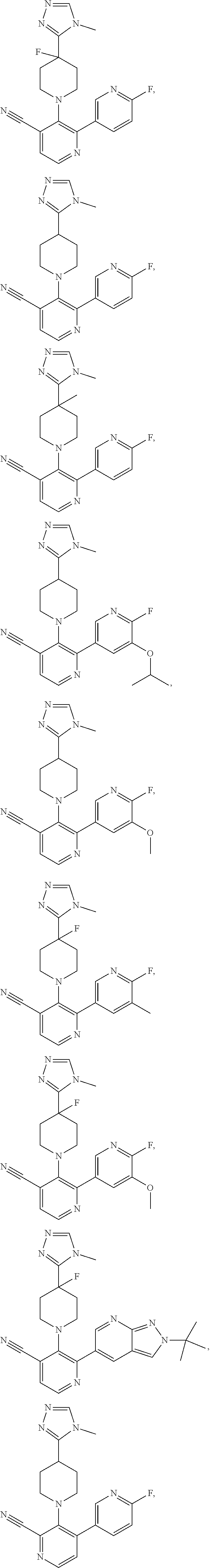 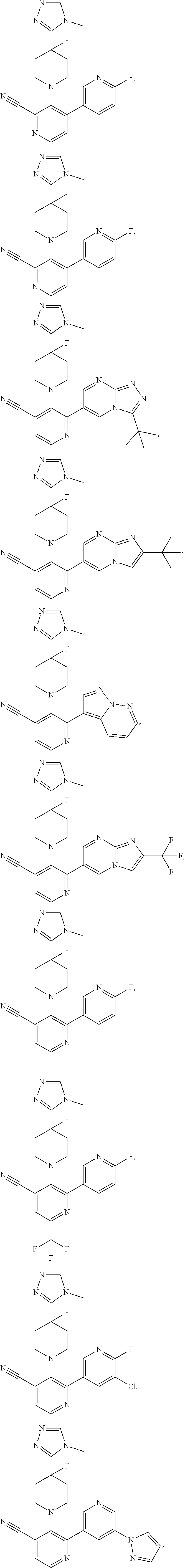 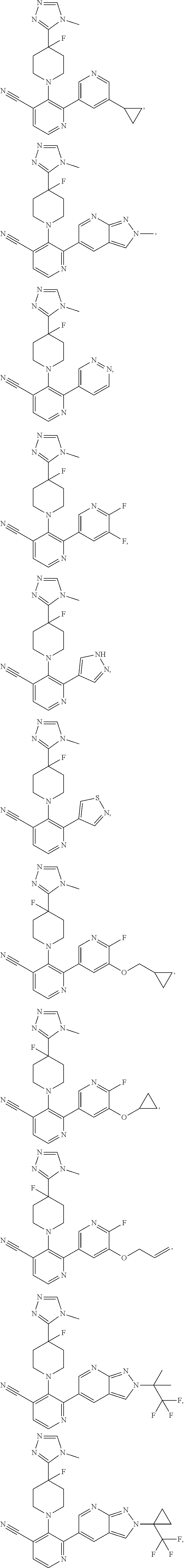 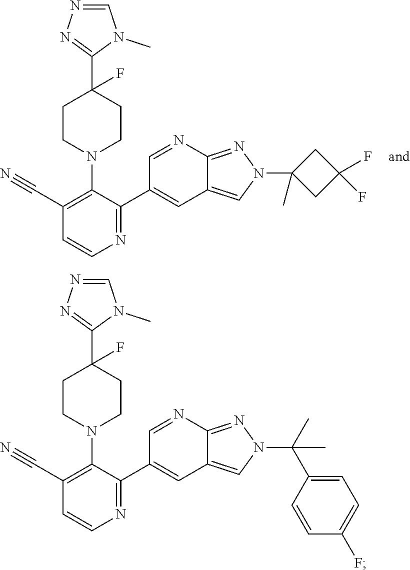
or a salt thereof.

9. A pharmaceutically acceptable salt of a compound according to claim 1.

10. A pharmaceutical composition comprising one or more compounds according to claim 1, or pharmaceutically acceptable salts thereof, together with one or more inert carriers and/or diluents.

11. A pharmaceutical composition comprising one or more compounds according to claim 1, or pharmaceutically acceptable salts thereof, and one or more additional therapeutic agents, together with one or more inert carriers and/or diluents.

12. The pharmaceutical composition according to claim 11 wherein the one or more additional therapeutic agents are selected from the group consisting of anticancer agents and antifibrotic agents.

13. (canceled)

14. A method for the treatment of diseases, such as cancer or fibrotic diseases, and conditions associated with these diseases, in a patient in need thereof, the method being characterized in that one or more compounds according to claim 1 or pharmaceutically acceptable salts thereof are administered to the patient.

15. (canceled)
